SEQUENCE LISTING (6 sequences)

SEQ ID NO: 1 - DNA, 20 bp, Artificial Sequence
  example primer
gggatgatgacgacctgcta

SEQ ID NO: 2 - DNA, 20 bp, Artificial Sequence
  example primer
tgtcgttgcttgtctctcct

SEQ ID NO: 3 - DNA, 20 bp, Artificial Sequence
  example primer
tgcagagacacagacagagg

SEQ ID NO: 4 - DNA, 20 bp, Artificial Sequence
  example primer
gccagtgaatgagtagcagc

SEQ ID NO: 5 - DNA, 19 bp, Artificial Sequence
  example primer
actcttccagccttccttc

SEQ ID NO: 6 - DNA, 20 bp, Artificial Sequence
  example primer
atctccttctgcatcctgtc

(19) United States
(12) Patent Application Publication
(10) Pub. No.: US 2022/0054494 A1
(43) Pub. Date: Feb. 24, 2022

(54) METHODS FOR TREATING BLADDER AND URETHRA DYSFUNCTION AND DISEASE

(71) Applicants: University of Pittsburgh - Of the Commonwealth System of Higher Education, Pittsburgh, PA (US); Vanderbilt University, Nashville, TN (US); The Trustees of The University of Pennsylvania, Philadelphia, PA (US)

(72) Inventors: Edwin Kerry Jackson, Pittsburgh, PA (US); Lori Ann Birder, Pittsburgh, PA (US); Amanda Sue Wolf-Johnston, Pittsburgh, PA (US); Roger Dmochowski, Nashville, TN (US); Alan J. Wein, Philadelphia, PA (US)

(73) Assignees: University of Pittsburgh - Of the Commonwealth System of Higher Education, Pittsburgh, PA (US); Vanderbilt University, Nashville, TN (US); The Trustees of The University of Pennsylvania, Philadelphia, PA (US)

(21) Appl. No.: 17/434,894

(22) PCT Filed: Mar. 13, 2020
(86) PCT No.: PCT/US2020/022697

§ 371 Date: Aug. 30, 2021

Related U.S. Application Data
(60) Provisional application No. 62/817,859, filed on Mar. 13, 2019.
(60) Provisional application No. 62/877,220, filed on Jul. 22, 2019.

(51) Int. Cl. A61K 31/522; A61K 9/00; A61P 13/10

(52) U.S. Cl. CPC A61K 31/522; A61P 13/10; A61K 9/0034

(57) ABSTRACT

Methods of treating bladder or urethra dysfunction or disease in a subject and methods of increasing bladder smooth muscle contractility or increasing bladder wall volume in a subject are disclosed. In some examples, a purine nucleoside phosphorylase (PNPase) inhibitor or purine nucleoside substrate is administered, such as 8-aminoguanine or forodesine.

### CROSS REFERENCE TO RELATED APPLICATION

This claims the benefit of U.S. Provisional Application No. 62/817,859, filed Mar. 13, 2019, and U.S. Provisional Application No. 62/877,220, filed Jul. 22, 2019, which are both incorporated herein by reference in their entirety.

### ACKNOWLEDGMENT OF GOVERNMENT SUPPORT

This invention was made with government support under grant number AG050408, awarded by the National Institutes of Health. The government has certain rights in the invention.

### FIELD

This relates to the field of purine nucleoside phosphorylase (PNPase) inhibitors and PNPase purine nucleoside substrates, specifically to their use for treating bladder and urethra dysfunction or disease.

### BACKGROUND

Aging and its effect on the lower urinary tract is complex. Studies in animals show that multiple components in the bladder become dysfunctional with age. Aging impacts bladder mucosal, muscular, stromal, and neural components in differing manners and to differing extents. Common pathological impacts likely arise from vascular changes (ischemia associated with reperfusion injury, which is similar to that seen in the myocardium), mucosal pathology (increase in mucosal permeability and loss of mucosal cells with effects on the local cell-cell and cell-interstitium communications necessary for normal sensation and regulation of bladder wall inflammation), and the inability of the bladder musculature to exhibit normal compliance during filling and storage and normal contractility during emptying (resulting from vascular, neural, and other pathologic factors). Lower urinary tract dysfunction is a common pathology of the lower urinary tract in the elderly and frail, which includes a group of syndromes that share symptom types and result in similar clinical scenarios in this population. Common findings in this group include poor bladder contraction during emptying (such as underactive bladder syndrome, UAB), overactive bladder during filling and storage (often coexistent with UAB during emptying), and varying types of urinary incontinence (such as stress, urgency, and spontaneous), which arise from a combination of bladder and urethral dysfunctions. The demographics of these disorders suggest a rapid increase in occurrence of these symptoms and their underlying causative etiologies beginning in the fifth decade in both genders, increasing until end of life, and effecting at least 30% in aggregate of the post-50 year-old population. A need remains for treatments for and prevention of bladder and urethra dysfunction, such as in elderly subjects.

### SUMMARY

It is disclosed herein that PNPase inhibitors or a PNPase purine nucleoside substrates are of use for treating bladder or urethra dysfunction or disease in a subject.

In some embodiments, the methods include selecting a subject with bladder or urethra dysfunction or disease, and administering to the subject a therapeutically effective amount of a PNPase inhibitor or a PNPase purine nucleoside substrate. In some non-limiting examples, a subject is selected that has bladder dysfunction and has at least one of increased void volume, decreased void efficiency, decreased void frequency, increased bladder capacity, increased bladder storage, increased bladder wall volume, decreased sensitivity to stimuli (such as tactile stimuli, for example, abdominal or somatic stimuli), increased bladder ischemia, increased oxidative stress in the bladder, increased mitochondrial dysfunction in the bladder, or decreased bladder contractility (such as during emptying), as compared with a subject without the bladder dysfunction. In other embodiments, a subject is selected that that has urethra dysfunction and has one or more of increased oxidative stress in the urethra, increased mitochondrial dysfunction in the urethra, or decreased urethra contractility, as compared with a subject without the urethra dysfunction. In further embodiments, a subject is selected that has a bladder disease, such as cystitis (for example, interstitial cystitis, bladder pain syndrome, and/or radiation-induced cystitis).

In other embodiments, methods are disclosed for increasing bladder smooth muscle contractility or bladder wall volume in a subject. These methods include selecting the subject in need of increased bladder smooth muscle contractility or decreased bladder wall volume, and administering to the subject a therapeutically effective amount of a PNPase inhibitor or PNPase purine nucleoside substrate, thereby increasing bladder smooth muscle contractility or decreasing bladder wall volume in a subject.

In yet other embodiments methods are disclosed for improving urethral function in a subject. These method include selecting a subject with urethra dysfunction, and administering to the subject a therapeutically effective amount of a PNPase inhibitor or a PNPase purine nucleoside substrate, wherein administration of the PNPase inhibitor or a PNPase purine nucleoside substrate: a) improves the morphology of the smooth or striated muscle in the urethra; b) decreases disruption of mitochondria in the urethra; or c) increases expression of alpha smooth muscle actin (α-SMA) and cathepsin B in the urethra, thereby improving urethral function in the subject.

In certain non-limiting examples, a PNPase transition state analog is used in the disclosed methods.

The foregoing and other objects, features, and advantages of the invention will become more apparent from the following detailed description, which proceeds with reference to the accompanying figures.

### BRIEF DESCRIPTION OF THE DRAWINGS

FIGS. 1A-1C: 8-Aminoguanine (8-AG) attenuates age-related changes in voiding frequency (FIG. 1A), change in intercontraction interval (FIG. 1B), and increases in voided volume (FIG. 1C). Values represent means and SEMs. **Indicates p<0.01. ***Indicates p<0.001. ****Indicates p<0.0001.

FIGS. 2A-2B: Aging decreases tactile abdominal (FIG. 2A) and somatic (FIG. 2B) tactile mechanical sensitivity, which is restored to a younger state by 8-AG treatment. Values represent means and SEMs.

FIG. 3: Aging increases bladder wall volume which is restored to a younger state by 8-AG treatment. Values represent means and SEMs. **Indicates p<0.01. ***Indicates p<0.001.

FIGS. 4A-4C: Representative transmission electron microscopy images of bladder smooth muscle in young rats (FIG. 4A), aged rats (FIG. 4B), and aged rats treated with 8-AG; FIG. 4C). These images reveal abnormal detrusor smooth muscle morphology in aged rats, which includes separation and degeneration of cells. However, the abnormal morphology in aged rats is restored to a younger state by 8-AG treatment.

FIGS. 5A-5C: High magnification transmission electron microscopy images of bladder smooth muscle in young rats (FIG. 5A), aged rats (FIG. 5B), and aged rats treated with 8-AG; FIG. 5C). Higher magnification images reveal significant swelling and disruption of smooth muscle mitochondria in aged bladders; these anomalies were restored to a younger state by 8-AG treatment.

FIGS. 6A-6C: Aging increases the (FIG. 6A) senescence marker p16 (p16INK4a, cyclin-dependent kinase inhibitor 2A) in bladder smooth muscle, which is restored to a younger state by 8-AG treatment. Aging also increases both V1a (FIG. 6B) and V2 vasopressin (FIG. 6C) receptors in the rat kidney, which is restored to a younger state by 8-AG treatment. Values represent means and SEMs. **Indicates p<0.01. ***Indicates p<0.001.

FIGS. 7A-7C: Confocal microscopy coupled with perfusion of the bladder with fluorophores shows increased tortuosity with appearance of ischemia in aged vessels (FIG. 7B) compared with young vessels (FIG. 7A), which is restored to a younger state by 8-AG treatment (FIG. 7C).

FIGS. 8A-8F shows the effect of oral 8-AG on urethral smooth muscle and external sphincter (striated muscle) and mitochondria. Aging is associated with urethral smooth and striated muscle and mitochondrial degeneration, which is restored to a younger state by 8-AG. Representative smooth muscle (SM) transmission electron microscopy (TEM) images (n=3 each young, aged, and aged+AG treated) revealed significant alterations in SM, which showed separation and degeneration of cells and mitochondria which were severely swollen and disrupted (arrows) as compared to younger tissue. In contrast, 8-AG treatment of aged rats restored SM anomalies and promoted healthy mitochondria to levels similar to the young state. FIGS. 8A-8C are images of urethral smooth muscle from young, aged, and aged+8-AG-treated animals. FIGS. 8D-8F are images of external urethral sphincter (striated muscle) from young, aged, and aged+8-AG-treated animals.

FIGS. 9A-9B show the effect of oral 8-AG treatment on LC3 (lipidated form) in aged detrusor (FIG. 9A) and on cathepsin B in aged urethra (FIG. 9B). Aged bladder detrusor layer exhibit decreased expression of LC3 (which correlates with induction of autophagy, a process for removing damaged cells and organelles) as compared to young rats and 8-AG treatment partially restores expression toward younger state (FIG. 9A). Similar changes in cathepsin B expression (suggesting a decrease in lysosomal function and decreased removal of cellular debris) were detected in aged urethra as compared to young rats with a trend toward reversal with oral 8-AG treatment (FIG. 9B) (n=7 for young and aged groups; n=5 for aged+8-AG treatment group). Values represent means and SEMs.

FIGS. 10A-10B show the effect of oral 8-AG treatment on bladder and urethral (α-SMA) expression. Aged bladders exhibit decreased expression of the smooth muscle (SM) marker, α-SMA in both detrusor (FIG. 10A) and urethra (FIG. 10B) as compared to young rats with 8-AG treatment increasing expression toward younger state. (n=4 each; young, aged, aged+8-AG treatment). Values represent means and SEMs.

FIGS. 11A-11F show the effect of intravesical 8-AG on bladder smooth muscle mitochondrial architecture. Representative transmission electron microscopy (TEM) images (n=1 each, control aged and aged+8-AG treated) revealed significant alterations in detrusor smooth muscle mitochondria with regions of swelling and disruption in aged (untreated) rats. In contrast, intravesical 8-AG treatment of aged rats normalized the mitochondrial abnormalities. Scale bars (FIGS. 11A and 11D=1 μm; FIGS. 11B-11C, 11E, and 11F=600 nm).

FIG. 12 shows the effect of oral 8-AG on the mechanical response to bladder stretch. Representative figure indicates mechanical stress in the bladder wall as a function of increased stretch for aged (curve 1) and young (curve 4) rats as well as in aged rats treated with 8-amino guanine for 1 week (curve 2) and 4 weeks (curve 3). The bladders showed an initial soft or compliant response until a critical stretch is reached at which the bladder rapidly stiffens. This critical stretch at which the steep increase in stiffness occurs was substantially lower in aged rats and was recovered to levels found in young rats following treatment with 8-AG (n=3 rats each, young, aged, and aged+8-AG).

FIGS. 13A-13D show the effect of oral 8-AG on loss of collagen fiber tortuosity in the lamina propria layer. The top row shows representative projected multiphoton images of a bladder wall (n=3 animals each; lamina propria layer) showing collagen fibers. The graph indicates the first transition phase at which physiological stretch results in an ‘unfolding’ of the lamina propria/urothelium due to collagen fiber recruitment in young, aged, and aged plus 8-AG treated rats. Normalizing the lamina propria collagen fiber tortuosity with 8-AG treatment indicates that the lamina propria/urothelium becomes more undulated, which may serve as a protective measure from excessive mechanical stress during the early (physiologic) bladder filling phase. Values represent means and SEMs.

FIGS. 14A-14D show the effect of oral 8-AG on loss of collagen fiber tortuosity in the detrusor layer. The top row shows representative projected multiphoton images of a bladder wall (n=3 animals each; detrusor smooth muscle layer) showing collagen fibers. Young bladders show a broad distribution of fiber tortuosity that can be seen in the MPM images as wavy collagen fibers. This tortuosity enables expansion of the bladder at low loads as fibers are recruited. With age, the tortuosity is diminished corresponding to a substantial fraction of highly straightened fibers that inhibit expansion. After treatment, fiber tortuosity is increased, corresponding to a larger fraction of highly tortuous fibers and a more extensible bladder. The graph indicates the phase at which stretch results in a rapid bladder stiffness due to the tortuosity of collagen fibers young, aged, and aged+8-AG treated rats. Values represent means and SEMs.

FIGS. 15A-15C show the effect of intravesical 8-AG on bladder function in aged rats, in which intravesical 8-AG attenuates age-related changes in bladder functions. The intravesical treatment attenuates age-related changes in voiding frequency (FIG. 15A), inter-contraction interval (FIG. 15B), and voided volume (FIG. 15C). N=2 each aged, aged+8-AG treatment with multiple assays performed per animal. Values represent means and SEMs.

FIGS. 16A-16B show the effect of intravesical 8-AG on abdominal and tactile mechanical sensitivity, in which intravesical 8-AG attenuates age-related changes in tactile sensitivity. This treatment improves the age-associated decrements in tactile abdominal (FIG. 16A) and somatic (FIG. 16B) tactile mechanical sensitivity. N=2 each aged, aged+8-AG treatment with multiple assays performed per animal. Values represent means and SEMs.

FIGS. 17A-17C show the effect of intravesical forodesine on bladder function (3 weeks of treatment), in which intravesical forodesine significantly attenuates age-related changes in bladder functions. The intravesical treatment significantly attenuates age-related changes in voiding frequency (FIG. 17A), changes in inter-contraction interval (FIG. 17B), and increases in voided volume (FIG. 17C) in n=2 each (aged control and aged+Forodesine treated). Values represent means and SEMs. *Indicates p<0.05. **Indicates p<0.01.

FIGS. 18A-18C show urinary levels of endogenous 8-AG, hypoxanthine, and isoprostanes in aged rats. FIG. 18A shows that aged rats excrete zero endogenous 8-AG, which is restored to younger levels following 8-AG treatment (n=4 rats each). FIG. 18B shows that aging is associated with a significant increase (n=4 samples/group) in urinary levels of hypoxanthine versus young animals. Treatment of aged rats with 8-AG normalizes urinary hypoxanthine levels to that of young animals. FIG. 18C is a representative graph showing that intravesical instillation of hypoxanthine (30 μM) increases urinary isoprostanes (measure of oxidative stress) to a greater extent in aged versus young rats. Similar changes in urinary isoprostanes in aged rats were also observed with a higher (100 μM; n=1) hypoxanthine concentration. Values represent means and SEMs.

FIGS. 19A-19C show urinary levels of isoprostanes, hypoxanthine, and xanthine in patients with voiding symptoms as compared with healthy controls. FIG. 19A shows significant elevation of urinary 8-isoprostanes in patients with voiding symptoms (n=6) versus healthy controls (n=11); FIG. 19B shows elevated urinary levels of hypoxanthine; and FIG. 19C shows xanthine in patients with voiding symptoms (n=7) versus healthy controls (n=4). Values represent means and SEMs.

FIGS. 20A-20C: 8-AG attenuates age-related decreases in voiding frequency (FIG. 20A) and increases in intercontraction interval (FIG. 20B) and voided volume (FIG. 20C). Numbers in bars indicate sample size. Values represent means and SEMs. *Indicates p<0.05. **Indicates p<0.01. ***Indicates p<0.001. ****Indicates p<0.0001.

FIGS. 21A-21B: Aging decreases abdominal (FIG. 21A) and somatic (FIG. 21B) responses to tactile mechanical stimuli, which is restored to a younger state by 8-AG treatment. Numbers in bars indicate sample size. Values represent means and SEMs. *Indicates p<0.05. **Indicates p<0.01. ***Indicates p<0.001.

FIGS. 22A-22D: Ribbon-scanning confocal microscopy coupled with perfusion of the bladder with fluorophores shows increased vascular tortuosity with appearance of reduced perfusion in aged bladders (FIG. 22B) compared with young bladders (FIG. 22A), which is restored to a younger state by 8-AG treatment (FIG. 22C). FIG. 22D shows doppler flowmeter measurements that reveal a significant decrease in bladder blood flow in aged compared to young rats; blood flow defects in the old bladder are reversed to a younger state with 8-AG treatment. Numbers in bars indicate sample size. Values represent means and SEMs. *Indicates p<0.05.

FIGS. 23A-23D: Western immunoblotting revealed significant aging-associated changes in proteins linked to mitochondrial dynamics and quality control within the bladder mucosa. These changes include mitofusin 2 (MFN2, FIG. 23A), a protein involved in mitochondrial fusion; dynamin-related protein (DRP-1, FIG. 23B), which is involved in mitochondrial fission; parkin (FIG. 23C), which plays a role in mitophagy; and cleaved caspase 3 (FIG. 23D), which is activated upon initiation of apoptosis. In all cases, treatment with 8-AG restored changes similar to a younger state. Numbers in bars indicate sample size. Values represent means and SEMs. *Indicates p<0.05. **Indicates p<0.01.

FIGS. 24A-24F: Concurrent imaging and mechanical testing of bladder collagen fibers showed a significant decrease in tortuosity in the detrusor layer of aged bladders (FIG. 24B) compared to young bladders (FIG. 24A). Aging fibers are prematurely recruited (straightened) at a lower degree of stretch as compared to collagen fibers in younger bladders (depicted in FIG. 24D). This change (corresponding to increased bladder stiffness) was restored with 8-AG treatment (FIGS. 24C-24D). These changes lead to earlier steepening of stress/stretch curve in aged versus young bladders (FIG. 24E) corresponding to increased bladder stiffness. In addition, aged bladders exhibit increased bladder wall width as compared to younger rat bladders. Bladder wall thickness in old rats was restored to a younger state with 8-AG treatment (FIG. 24F). Numbers in bars indicate sample size. Values represent means and SEMs. *Indicates p<0.05. **Indicates p<0.01.

FIGS. 25A-25D: Concurrent imaging and mechanical testing of bladder collagen fibers showed a significant decrease in tortuosity in the lamina propria in aged bladders (FIG. 25B) compared to young bladders (FIG. 25A). Aging fibers are prematurely recruited (straightened) at a lower degree of stretch as compared to collagen fibers in younger bladders (termed critical stretch and depicted in FIG. 25D). This change (corresponding to increased bladder stiffness) was restored with 8-AG treatment (FIGS. 25C-25D). Numbers in bars indicate sample size. Values represent means and SEMs. *Indicates p<0.05.

FIGS. 26A-26F: Representative transmission electron microscopy images of bladder smooth muscle in young rats (FIG. 26A), aged rats (FIG. 26B), and aged rats treated with 8-AG (FIG. 26C). These images reveal abnormal detrusor smooth muscle morphology in aged rats, which includes separation and degeneration of cells. However, the abnormal morphology in aged rats is restored to a younger state by 8-AG treatment. FIG. 26D-26F depicts higher magnification transmission electron microscopy images that reveal significant swelling and disruption of smooth muscle mitochondria in aged bladders (FIG. 26E) compared with young bladders (FIG. 26D); these anomalies were restored to a younger state by 8-AG treatment (FIG. 26F).

FIGS. 27A-27F: Aged bladder detrusor smooth muscle exhibit alterations in α-SMA (FIG. 27A) and hydroxyproline (FIG. 27B), an indicator of fibrosis. Significant changes were detected in senescent biomarker p16 (FIG. 27C), catalase activity (FIG. 27D), cleaved caspase 3 (FIG. 27E), and cleaved PARP (FIG. 27F). 8-AG treatment restored these biomarkers to that of a younger state. Numbers in bars indicate sample size. Values represent means and SEMs. *Indicates p<0.05.

FIGS. 28A-28F: Shown are representative transmission electron microscopy images of urethral smooth muscle in young rats (FIG. 28A), aged rats (FIG. 28B) and aged rats treated with 8-AG (FIG. 28C). The damaged smooth muscle and abnormal mitochondrial morphology in aged rats is restored to a younger state by 8-AG treatment. FIGS. 28D-28F depict age-associated changes in urethral nitrotyrosine (FIG. 28D) as well as α-SMA (FIG. 28E) and significant changes in cleaved PARP (FIG. 28F), all of which were normalized to a younger state with 8-AG treatment. Numbers in bars indicate sample size. Values represent means and SEMs. *Indicates p<0.05. ***Indicates p<0.001.

FIGS. 29A-29E: Depicted are representative transmission electron microscopy images of external urethral sphincter (EUS, striated muscle) in young rats (FIG. 29A), aged rats (FIG. 29B) and aged rats treated with 8-AG (FIG. 29C). FIGS. 29D-29E depict significant age associated changes in EUS cleaved caspase 3 (FIG. 29D) and cleaved PARP (FIG. 29E); all of these abnormalities in aged rats are restored to a younger state by 8-AG treatment. Numbers in bars indicate samples size. Values represent means and SEMs. *Indicates p<0.05. **Indicates p<0.01.

FIGS. 30A-30C: In aged rats, endogenous urinary 8-AG is below assay detection limits (FIG. 30A); yet aged rats have higher urinary hypoxanthine levels (FIG. 30B); both of these abnormalities are restored to younger levels with 8-AG treatment. In addition, guanosine levels (FIG. 30C) are altered with age and recovered with 8-AG treatment. Values represent means and SEMs. *Indicates p<0.05.

FIG. 31A-31B: Old patients with bladder disease have elevated levels of urinary hypoxanthine (FIG. 31A) and xanthine (FIG. 31B) as compared to young, healthy controls. Numbers in bars indicate sample size. Values represent means and SEMs. *Indicates p<0.05.

FIGS. 32A-32C: 8-AG attenuates cyclophosphamide (CYP)-induced increases in voiding frequency (FIG. 32A) and decreases in intercontraction interval (FIG. 32B). FIG. 32C shows doppler flowmeter measurements that reveal an increase in bladder blood flow (hyperemia) in CYP-treated compared to untreated control rats; blood flow defects in the CYP-inflamed bladder were reversed to a control state with 8-AG treatment. Numbers in bars indicate sample size. Values represent means and SEMs. *Indicates p<0.05.

FIGS. 33A-33B: RT-qPCR reveals mRNA expression for the inflammatory cytokines IL-1beta (FIG. 33A) and MCP-1 (FIG. 33B) in bladder tissue obtained from CYP-treated rats. However, in CYP-treated rats co-administered with 8-AG, the expressions of IL-1beta and MCP-1 mRNA were nearly abolished. Numbers in bars indicate sample size. Values represent means and SEMs. * Indicates p<0.05.

FIGS. 34A-34B: The graph (FIG. 34A) shows the abdominal mechanical pain threshold in rats (using Von Frey filaments) in control (baseline) rats versus rats treated with CYP and CYP+8-AG. FIG. 34B depicts representative images of intact bladders that reveal significant inflammation and bleeding in CYP-treated rats which was nearly abolished by oral treatment with 8-AG. Values represent means and SEMs. *Indicates p<0.05. **Indicates p<0.01.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file, created on Mar. 13, 2020, 1.57 KB, which is incorporated by reference herein. In the accompanying sequence listing:

SEQ ID NOS: 1 and 2 are exemplary IL-1beta primers.

SEQ ID NOS: 3 and 4 are exemplary MCP-1 primers.

SEQ ID NOS: 5 and 6 are exemplary β-actin primers.

### DETAILED DESCRIPTION

Peroxynitrite (ONOO—) is formed in vivo from the diffusion-controlled reaction between superoxide anion (O₂.⁻) and nitric oxide (NO) (Carballal S et al., Biochimica et Biophysica Acta, 1840:768-780, 2014). Further, ONOO— is a highly reactive nitrogen species (RNS) that can mediate nitration (such as insertion of —NO₂) of numerous endogenous compounds, including those containing a guanine moiety (Ohshima H et al., Antioxidants & Redox Signaling, 8:1033-1045, 2006; Szabo C et al., Nitric Oxide, 1:373-385, 1997; Yermilov V et al., FEBS Lett, 376:207-210, 1995). In this regard, ONOO— nitrates guanine moieties at position 8 of the purine ring to produce 8-nitroguanine units in DNA, RNA, and the guanine nucleotide pool (Ohshima H et al., Antioxidants & Redox Signaling, 8:1033-1045, 2006; Szabo C et al., Nitric Oxide, 1:373-385, 1997; Yermilov V et al., FEBS Lett, 376:207-210, 1995). Free guanine may be nitrated at the 8 position. In addition to RNS-mediated modification of guanine-containing compounds, reactive oxygen species (ROS), such as O₂.⁻, can also modify position 8 of guanine moieties by inserting a hydroxyl functional group (Szabo C et al., Nitric Oxide, 1:373-385, 1997; Misiaszek R et al., Journal of Biological Chemistry, 279:32106-32115, 2004).

After modification of guanine moieties by RNS or ROS, subsequent catabolism of RNA, DNA, and the guanine nucleotide pool will release 8-nitroguanosine, 8-nitro-2-deoxyguanosine, 8-hydroxyguanosine, and 8-hydroxy-2-deoxyguanosine. Reduction of 8-nitro groups could yield 8-aminoguanosine and 8-amino-2-deoxyguanosine, and PNPase can convert such compounds into 8-AG (Osborne W R et al., Immunology, 59:63-67, 1986). In addition, PNPase may convert 8-nitroguanosine and 8-nitro-2-deoxyguanosine into 8-nitroguanine, and reduction of 8-nitroguanine would yield 8-AG. Similarly, PNPase may produce 8-hydroxyguanine from 8-hydroxyguanosine or 8-hydroxy-2-deoxyguanosine. Consistent with this framework are studies confirming the presence of 8-nitroguanosine, 8-aminoguanosine, 8-AG, 8-hydroxyguanosine, 8-nitroguanine, 8-hydroxyguanine, and 8-hydroxy-2-deoxyguanosine in tissues or urine (Akaike T et al., Proc Natl Acad Sci USA, 100:685-690, 2003; Sodum R S et al., Chem Res Toxicol, 6:269-276, 1993; Park E M et al., Proc Natl Acad Sci USA, 89:3375-3379, 1992; Ohshima H et al., Antioxid Redox Signal, 8:1033-1045, 2006; Fraga C G et al., Proc Natl Acad Sci USA, 87:4533-4537, 1990; Lam P M et al., Free Radic Biol Med, 52:2057-2063, 2012).

PNPase transition state analogs are also of use in the disclosed methods. In some non-limiting examples, the transition state analog can be forodesine or a derivative thereof (such as DADMe-immucillin-H, DATMe-immucillin-H, or SerMe-immucillin-H) or pharmaceutically acceptable salt thereof (such as a PNPase transition state analog chloride salt), or as described in U.S. Pat. Nos. 4,985,433; 4,985,434, 5,008,265; 5,008,270; 5,565,463, and 5,721,240 as well as US Published Patent Application No. 2018/0258091A1, all of which are incorporated herein by reference in their entireties. Pharmaceutically acceptable salts of these compounds are also of use.

Methods are disclosed herein that utilized PNPase inhibitors. In some embodiments, these methods include selecting a subject with bladder dysfunction or urethra dysfunction, and treating the subject with a therapeutically effective amount of a PNPase inhibitor or a PNPase purine nucleoside substrate, thereby treating the bladder dysfunction or urethra dysfunction.

### I. Terms

Unless otherwise noted, technical terms are used according to conventional usage. Definitions of common terms in molecular biology can be found in Krebs et al (Eds.), Lewin's Genes XII, published by Jones & Bartlett Publishers, 2017; and Meyers et al. (eds.), The Encyclopedia of Cell Biology and Molecular Medicine, published by Wiley-VCH in 16 volumes, 2008; and other similar references.

As used herein, the singular forms “a,” “an,” and “the” refer to both the singular as well as plural unless the context clearly indicates otherwise. Further, “or” also include “and/or”; thus, “a bladder dysfunction or a urethra dysfunction” also includes “a bladder dysfunction and/or a urethra dysfunction,” and compositions of use in the methods herein can be used alone or in combination. Although many methods and materials similar or equivalent to those described herein can be used, particular suitable methods and materials are described below. The term “comprises” means “includes.” The term “about” means within five percent, unless otherwise indicated. All publications, patent applications, patents, and other references mentioned herein are incorporated by reference in their entirety. In case of conflict, the present specification, including explanations of terms, will control. In addition, the materials, methods, and examples are illustrative only and not intended to be limiting. To facilitate review of the various embodiments, the following explanations of terms are provided.

Administration: To provide or give a subject an agent (such as a PNPase transition state analog or a guanine, guanosine, inosine, or hypoxanthine comprising a substituent at the 8-position) by any effective route. Exemplary routes of administration include, but are not limited to, oral, injection (such as subcutaneous, intramuscular, intradermal, intraperitoneal, intravenous, and intratumoral), sublingual, rectal, transdermal, intranasal, vaginal, instillation into the bladder, administration to the urethra or bladder, and inhalation routes.

Agent: Any polypeptide, compound, small molecule, organic compound, salt, polynucleotide, or other molecule of interest. Agent can include a therapeutic agent, a diagnostic agent or a pharmaceutical agent. A therapeutic agent is a substance that demonstrates some therapeutic effect by restoring or maintaining health, such as by alleviating the symptoms associated with a disease or physiological disorder, or delaying (including preventing) progression or onset of a disease, such as, but not limited to, bladder dysfunction or urethra dysfunction.

Aliphatic: A hydrocarbon, or a radical thereof, having at least one carbon atom to 50 carbon atoms, such as one to 25 carbon atoms, or one to ten carbon atoms, and which includes alkanes (or alkyl), alkenes (or alkenyl), alkynes (or alkynyl), including cyclic versions thereof, and further including straight- and branched-chain arrangements, and all stereo and position isomers as well.

Alkenyl: An unsaturated monovalent hydrocarbon having at least two carbon atoms to 50 carbon atoms, such as two to 25 carbon atoms, or two to ten carbon atoms, and at least one carbon-carbon double bond, wherein the unsaturated monovalent hydrocarbon can be derived from removing one hydrogen atom from one carbon atom of a parent alkene. An alkenyl group can be branched, straight-chain, cyclic (such as cycloalkenyl), cis, or trans (such as E or Z).

Alkyl: A saturated monovalent hydrocarbon having at least one carbon atom to 50 carbon atoms, such as one to 25 carbon atoms, or one to ten carbon atoms, wherein the saturated monovalent hydrocarbon can be derived from removing one hydrogen atom from one carbon atom of a parent compound (such as alkane). An alkyl group can be branched, straight-chain, or cyclic (such as cycloalkyl).

Alkoxyl: A univalent radical R—O—, or anion R—O—, wherein R is an alkyl group.

Alkynyl: An unsaturated monovalent hydrocarbon having at least two carbon atoms to 50 carbon atoms, such as two to 25 carbon atoms, or two to ten carbon atoms and at least one carbon-carbon triple bond, wherein the unsaturated monovalent hydrocarbon can be derived from removing one hydrogen atom from one carbon atom of a parent alkyne. An alkynyl group can be branched, straight-chain, or cyclic (such as cycloalkynyl).

α-SMA: Also known as aortic smooth muscle actin, alpha-actin 2, ACTA2, alpha aortic smooth muscle actin, ACTSA, and vascular smooth muscle actin (for example, OMIM no. 102620), α-SMA is involved in involved in cell motility, structure, and integrity. α-SMA nucleic acids and proteins are included. Exemplary α-SMA mRNA and proteins include GENBANK® sequences BC017554.2 and AAH93052.1, respectively, Jul. 17, 2019, both incorporated by reference herein in their entireties. Other α-SMA molecules are possible. One of ordinary skill in the art can identify additional α-SMA nucleic acid and protein sequences, including α-SMA variants that retain biological activity (such as involvement in cell motility, structure, and integrity).

Amide: —NC(O)R, wherein R is hydrogen, aliphatic, aryl, heteroaliphatic, or heteroaryl.

Amine: —NR′R, wherein each of R and R′ independently are hydrogen, aliphatic, aryl, heteroaliphatic, or heteroaryl.

Analog: A compound with a molecular structure closely similar to that of another, such as an analog of the transition state of a substrate during catalysis (for example, a transition state analog of catalysis by PNPase).

Animal: Living multi-cellular vertebrate organisms, a category that includes, for example, mammals and birds. The term mammal includes both human and non-human mammals. Similarly, the term “subject” includes both human and veterinary subjects.

Aryl: An aromatic carbocyclic group comprising at least five carbon atoms to 15 carbon atoms, such as five to ten carbon atoms, having a single ring or multiple condensed rings, which condensed rings can or may not be aromatic provided that the point of attachment is through an atom of the aromatic carbocyclic group.

Bladder disease: Also known as urinary bladder disease, bladder disease is a medical condition associated with abnormal urinary or bladder structure or function that produces a negative outcome in a subject (such as a human or veterinary subject). Examples of bladder diseases include cystitis (such as interstitial cystitis), urinary incontinence, overactive bladder, or bladder cancer. In some examples, one or more bladder diseases are present simultaneously. For example, cystitis can include signs or symptoms of overactive bladder or urinary incontinence.

Bladder dysfunction: Also known as urinary bladder dysfunction, bladder dysfunction is impaired bladder function, such as impairment to urine storage or expulsion function or timing. Examples of bladder dysfunction in a subject include increased void volume, decreased void efficiency, decreased void frequency, increased bladder capacity, increased bladder storage, increased bladder wall volume, decreased sensitivity to stimuli, increased bladder ischemia, increased oxidative stress in the bladder, decreased smooth muscle contractility, or increased mitochondrial dysfunction in the bladder, as compared with a subject without the bladder dysfunction. In some examples, a subject with bladder dysfunction may experience urinary incontinence, overactive bladder, or underactive bladder. Bladder dysfunction can occur at any age, but is more common when the subject is an older adult, such as an adult at least 50 years old.

Carbonate: —OC(O)OR, wherein R is hydrogen, aliphatic, aryl, heteroaliphatic, or heteroaryl.

Carbonyl: C═O, wherein the carbon located at the 8 position of guanine or guanosine forms a double bond with an oxygen atom.

Carboxyl: —C(O)OR, wherein R is hydrogen, aliphatic, aryl, heteroaliphatic, or heteroaryl.

Cathepsin B: Also known as CTSB, CATB, amyloid precursor protein secretase, APP secretase, and APPS (for example, OMIM no. 116810, cathepsin B is a lysosomal cysteine protease and plays a role in intracellular proteolysis. cathepsin B nucleic acids and proteins are included. Exemplary cathepsin B mRNA and proteins include GENBANK® sequences L16510.1 and AAC37547.1, respectively, Jul. 17, 2019, both incorporated by reference herein in their entireties. Other cathepsin B molecules are possible. One of ordinary skill in the art can identify additional cathepsin B nucleic acid and protein sequences, including cathepsin B variants that retain biological activity (such as intracellular proteolysis).

Control subject: A control subject is a subject that is used to provide a basis for comparison. As a comparison to subjects with or at risk for a particular condition (such as a subject that has a bladder dysfunction), control subjects may belong to a group of healthy subject who are studied to observe how their symptoms, traits, or behaviors compare to a group of subjects with or at risk for a particular condition.

Cytokines: A broad category of small proteins (approximately 5-20 kDa) that are important in cell signaling. Their release has an effect on the behavior of cells around them. Cytokines are involved in autocrine signaling, paracrine signaling and endocrine signaling as immunomodulating agents. Cytokines include chemokines, interferons, interleukins, lymphokines, and tumor necrosis factors but generally not hormones or growth factors. Cytokines are produced by a broad range of cells, including immune cells, such as macrophages, B lymphocytes, T lymphocytes, and mast cells, as well as endothelial cells, fibroblasts, and various stromal cells; a given cytokine may be produced by more than one type of cell. Cytokines are important in health and disease, specifically in host responses to infection, immune responses, inflammation, trauma, sepsis, cancer, and reproduction. They act through receptors and are especially important in the immune system; cytokines modulate the balance between humoral and cell-based immune responses, and they regulate the maturation, growth, and responsiveness of particular cell populations. Cytokines include interleukins, such as IL-1beta, and chemoattractants, such as monocyte chemoattractant protein-1 (MCP-1).

Cystitis: A bladder disease that includes inflammation of the bladder. Specific examples of cystitis include acute cystitis (a sudden occurrence of cystitis) or interstitial cystitis (IC; also known as bladder pain syndrome, BPS), which is a chronic inflammatory bladder disease that causes bladder pain and frequent, urgent urination. The American Urological Association (AUA) guidelines panel defines bladder pain syndrome/interstitial cystitis as an unpleasant sensation (pain, pressure, discomfort) perceived to be related to the urinary bladder, associated with lower urinary tract (LUT) symptoms of more than six weeks duration, in the absence of infection or other identifiable causes. Signs and symptoms of overactive bladder and recurrent urinary tract infection can overlap with those of BPS/IC, but BPS/IC patients tend to experience less urinary incontinence and more dyspareunia. Radiation-induced cystitis, a complication of radiation therapy to pelvic tumors, results in inflammation of the bladder and urethra and often presents with similar symptoms.

Ester: —OC(O)R, wherein R is hydrogen, aliphatic, aryl, heteroaliphatic, or heteroaryl.

Halogen: bromo, fluoro, iodo, or chloro.

Heteroaliphatic: An aliphatic group comprising at least one heteroatom to 20 heteroatoms, such as one to 15 heteroatoms, or one to 5 heteroatoms, which can be selected from, but not limited to oxygen, nitrogen, sulfur, selenium, phosphorous, and oxidized forms thereof within the group.

Heteroalkyl/Heteroalkenyl/Heteroalkynyl: An alkyl, alkenyl, or alkynyl group (which can be branched, straight-chain, or cyclic) comprising at least one heteroatom to 20 heteroatoms, such as one to 15 heteroatoms, or one to 5 heteroatoms, which can be selected from, but not limited to oxygen, nitrogen, sulfur, selenium, phosphorous, and oxidized forms thereof within the group.

Heteroaryl: An aryl group comprising at least one heteroatom to six heteroatoms, such as one to four heteroatoms, which can be selected from, but not limited to oxygen, nitrogen, sulfur, selenium, phosphorous, and oxidized forms thereof within the ring. Such heteroaryl groups can have a single ring or multiple condensed rings, wherein the condensed rings may or may not be aromatic or contain a heteroatom, provided that the point of attachment is through an atom of the aromatic heteroaryl group.

Hydroxyl: —OH.

Incontinence: Incontinence is a loss of bladder or urethra control (such as an uncontrolled loss of urine). Any type of incontinence is included, such as stress, urgency (urge), spontaneous, overflow, or mixed incontinence. In some examples, the incontinence is stress incontinence, which can include urine leakage when pressure is exerted on the bladder or urethra (such as due to coughing, sneezing, laughing, exercising, or lifting a heavy object). In some examples, the incontinence is urgency (urge) incontinence, which can include an urge to urinate with a subsequent involuntary loss of urine. In some examples, the incontinence is spontaneous incontinence, such as loss of urine that is not associated with an urge. In some examples, the incontinence is overflow incontinence, such as a frequent or constant dribbling of urine (such as where the bladder does not fully empty). In some examples, the incontinence is mixed, such as more than one type of incontinence.

Inhibiting or treating a disease: Inhibiting the full development of a disease or condition, for example, in a subject who is at risk for a disease such as bladder dysfunction. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, reduced frequency of urination, increased void volume, a slower progression of the disease, an improvement in the overall health or well-being of the subject, or by other parameters well known in the art that are specific to the particular disease. “Prophylaxis” is a treatment administered to a subject who does not exhibit signs of a disease or exhibits only early signs for the purpose of decreasing the risk of developing pathology.

Interleukin 1-beta (IL-1β): Also known as IL1B, IL1-beta, leukocytic pyrogen, leukocytic endogenous mediator, mononuclear cell factor, and lymphocyte activating factor (e.g., OMIM 147720), IL-1β is a cytokine produced by activated macrophages is an important mediator of the inflammatory response. IL-Iβ is involved in a variety of cellular activities, including cell proliferation, differentiation, and apoptosis. Increased production and/or activity of IL-Iβ causes multiple autoinflammatory syndromes and has been linked to susceptibility to cancer and tuberculosis. Exemplary protein and nucleotide sequences for IL-Iβ are available at GENBANK® (e.g., Accession Nos. NP_000567.1 and NM_000576.3, respectively, incorporated by reference herein as available on Mar. 2, 2020).

Microtubule-associated protein 1 light chain 3 alpha (LC3): Also known as MAP1LC3A, microtubule-associated proteins 1a and 1b light chain 3, MAP1ALC3, MAP1BLC3, and LC3A (for example, OMIM 601242), LC3 plays a role in autophagy and protects cells from tumorigenesis. LC3 is activated through post-translational modification, including proteolysis and lipidation for membrane insertion. LC3 nucleic acids and proteins are included. Exemplary LC3 GENBANK® sequences include NM_181509.3 (mRNA) as well as NP_852610.1 and NP_115903.1 (protein sequences), incorporated by reference herein in their entireties as present on Jul. 17, 2019. Other LC3 molecules are possible. One of ordinary skill in the art can identify additional LC3 nucleic acid and protein sequences, including LC3 variant that retain biological activity (such as involvement in autophagy).

Monocyte chemotactic protein-1 (MCP-1): Also known as chemokine CC motif ligand 2 (CCL2); small inducible cytokine A2 (SCYA2); and monocyte chemotactic and activating factor (MCAF; e.g., OMIM 158105), MCP-1 is a cytokine that recruits monocytes to the site of tissue injury or infection and is involved in the pathogenesis of inflammatory disease. Exemplary protein and nucleotide sequences for IL-1$ are available at GENBANK® (e.g., Accession Nos. NP_002973.1 and NM_002982.4, respectively, incorporated by reference herein as available on Mar. 2, 2020).

Nitro: —NO₂.

Nitroso: —NO.

Nocturia: Interrupted sleep because of an urge to void, such as about two or more times per night.

Overactive bladder (OAB): OAB includes a frequent feeling of needing to urinate, for example, to a degree that it negatively affects one's life. In some examples, a loss of bladder control is concurrent (‘urge incontinence’). OAB can include a ‘wet’ variant, which is an urgent need to urinate with involuntary leakage, or a ‘dry’ variant, which is an urgent need to urinate but no involuntary leakage. Risk factors can include obesity, caffeine, constipation, and diabetes. Overactive bladder is characterized by a group of four symptoms: urgency, increased urinary frequency, nocturia, and urge incontinence.

Diagnosis may include a frequency/volume journal, cystourethroscopy, and questionnaires (such as general surveys of lower urinary tract symptoms and surveys specific to overactive bladder). Treatment can include pelvic floor exercises, bladder training, decreasing caffeine consumption, drinking moderate fluids, other behavioral methods, medications (such as anti-muscarinic medications, for example, darifenacin, hyoscyamine, oxybutynin, tolterodine, solifenacin, trospium, or fesoterodine), non-invasive electrical stimulation, botulinum toxin injection into the bladder, urinary catheter, surgery, or β3 adrenergic receptor agonists (such as mirabegron),

Pharmaceutically acceptable carrier: As used herein, the term “pharmaceutically acceptable carrier” includes any and all solvents, dispersion media, coatings, surfactants, antioxidants, preservatives (such as antibacterial agents, antifungal agents), isotonic agents, absorption delaying agents, salts, preservatives, drugs, drug stabilizers, binders, excipients, disintegration agents, lubricants, sweetening agents, flavoring agents, dyes, such like materials and combinations thereof, as would be known to one of ordinary skill in the art (see, such as Remington's Pharmaceutical Sciences, 1289-1329, 1990, incorporated herein by reference). Except insofar as any conventional carrier is incompatible with the active ingredient, its use in the therapeutic or pharmaceutical compositions is contemplated.

PNPase: Purine nucleoside phosphorylase, a glycosyltransferase, is an enzyme that catalyzes a chemical reaction between purine nucleoside (such as the PNPase purine nucleoside substrates inosine and guanosine) and phosphate. PNPase inhibitors inhibit the catalytic action of a PNPase. Examples of PNPase inhibitors include guanine and hypoxanthine; PNPase purine nucleoside substrates (such as inosine and guanosine); 8-substituted guanine, guanosine, inosine, and hypoxanthine; and PNPase transition state analogs, such as forodesine, or a forodesine derivative, for example, DADMe-immucillin-H, DATMe-immucillin-H, or SerMe-immucillin-H, or as described in U.S. Pat. Nos. 4,985,433; 4,985,434, 5,008,265; 5,008,270; 5,565,463, and 5,721,240 as well as US pat. pub. no. 2018/0258091A1, all of which are incorporated herein by reference in their entireties).

Subject: As used herein, the term “subject” refers to a mammal and includes, without limitation, humans and veterinary subjects, including domestic animals (such as dogs or cats), farm animals (such as cows, horses, or pigs), and laboratory animals (such as mice, rats, hamsters, guinea pigs, pigs, rabbits, dogs, or monkeys). In humans, and “older” subject is a subject that is more than 50 years of age.

Transition state analog: A chemical compound with a chemical structure that resembles the transition state of a substrate molecule in an enzyme-catalyzed chemical reaction. A ‘PNPase transition state analog’ is a compound that resembles the transition state of the reaction (such as catalysis of inosine to hypoxanthine or catalysis of guanosine to guanine) catalyzed by PNPase. These compounds act as an inhibitor of the PNPase by blocking its active site.

Therapeutically effective amount: The term “therapeutically effective amount” refers to the amount of an active ingredient (such as, but not limited to, a PNPase transition state analog, 8-substituted guanine, 8-substituted guanosine, 8-substituted guanosine, and 8-substituted inosine) that is sufficient to effect treatment when administered to a mammal in need of such treatment. The therapeutically effective amount will vary depending upon the subject and disease condition being treated, the weight and age of the subject, the severity of the disease condition, the manner of administration and the like, which can readily be determined by a prescribing physician.

Treating a disease: “Treatment” refers to a therapeutic intervention that ameliorates a sign or symptom of a disease or pathological condition, such as a sign or symptom of bladder dysfunction or urethra dysfunction. Treatment can also induce remission or cure of a condition or can reduce the pathological condition, such as decreasing void volume, decreasing frequency of urination, increasing smooth muscle contractility, reducing bladder ischemia, increasing urethra or bladder contractility, or a combination thereof. Prevention of a disease does not require a total absence of disease.

Underactive bladder: Also known as underactive bladder syndrome (UAB), underactive bladder includes difficulty with bladder emptying, such as hesitancy to start the stream, a poor or intermittent stream, or sensations of incomplete bladder emptying. Detrusor pressurization with strength or duration that is not sufficient for timely and efficient bladder emptying (‘detrusor underactivity or ‘DU’), bladder outlet obstruction, and volume hypersensitivity (‘OAB’) are often found concurrent with UAB. Various means can be used to diagnose UAB, including a subject or patient voiding diary (to assess voided volumes and frequency of voiding) and a post-void residual volume; uninstrumented uroflow as well as a neurologic and pelvic examination; imaging for abnormal bladder morphology or vesicoureteral reflux/hydronephrosis; or invasive urodynamics.

Urethra Dysfunction: Impaired urethra function, such as impairment to urine expulsion function or timing. Examples of urethra dysfunction in a subject include increased oxidative stress in the urethra, increased mitochondrial dysfunction in the urethra, or decreased urethra contractility. Urethra dysfunction can occur at any age, but is more common when the subject is an older adult, such as an adult at least 50 years old.

Urinary Frequency: The number of times of urination, such as in a day or night. “Increased urinary frequency” is eight or more times in a day, or two times or more in a night.

Urge Incontinence: Involuntary loss of urine occurring for no apparent reason while feeling urinary urgency.

Urgency (urination): A sudden, compelling desire to pass urine that is difficult to defer.

### II. Overview

Methods for using a PNPase inhibitor or a PNPase purine nucleoside substrate are disclosed herein. Many diseases in which 8-AG, 8-aminoguanosine, 8-hydroxyguanine, 8-hydroxyguanosine, 8-nitroguanine, 8-aminoinosine, 8-aminohypoxanthine, forodesine, and other PNPase inhibitors or substrates, such as other 8-substituted guanine, guanosine, inosine, and hypoxanthine compounds as well as other PNPase transition state analogs are useful are enriched in the elderly population. Disclosed herein are data examining safety in the elderly population, which disclose tolerance of chronic treatment with the PNPase inhibitor 8-AG in aged animals. The data show the unexpected finding that 8-AG improves bladder function in aged animals, reversing the effects of aging and restoring the bladder to a younger state. The data also show that bladder and urethra dysfunction and disease can be treated using a PNPase inhibitor or a PNPase purine nucleoside substrate. A comprehensive examination of the beneficial effects of this PNPase inhibitor on bladder dysfunction in aging animals is disclosed herein. The data further show that radiation-induced cystitis, bladder pain syndrome, and radiation-induced cystitis can be treated using a PNPase inhibitor or a PNPase purine nucleoside substrate.

In the aging population, millions of individuals in the US are affected by age-related lower urinary tract disorders (LUTD), including urgency, urinary incontinence, impaired contractility, nocturia, and decreased bladder sensation (often resulting in incomplete emptying). The urinary tract is particularly susceptible to the negative effects of age with increased prevalence of both storage and voiding symptoms above the age of 65 years. While some patients and health care providers may consider these conditions a normal part of aging, clearly these LUTD conditions are not normal and impair quality of life as well as place a substantial burden on healthcare resources. Despite the prevalence and consequences of these conditions in humans, many of these conditions continue to be undertreated. Thus, treatment of bladder and urinary tract dysfunction is an unmet medical need with limited to no available options, which can affect millions that suffer from age-related LUTD.

Some embodiments herein, PNPase inhibitor or purine nucleoside substrate are administered to a subject, and the administration reduces ischemia, oxidative stress, or mitochondrial dysfunction in the bladder of the subject; increases bladder contractility; decreases urinary incontinence; increases bladder smooth muscle contractility; or decreases bladder wall volume. In additional embodiments, administering the PNPase inhibitor or purine nucleoside substrate reduces oxidative stress or mitochondrial dysfunction in the urethra of the subject; increases urethra contractility; decreases urinary incontinence; increases urethra smooth muscle contractility; improves morphology of smooth or striated muscle in the urethra; decreases disruption of mitochondria in the urethra; or increases expression of α-SMA and cathepsin B in the urethra. In some examples, subjects are selected with bladder dysfunction or disease, such as decreased expression of α-SMA and microtubule-associated protein 1 light chain 3 alpha (LC3) in the bladder, as compared with a subject without the bladder dysfunction or disease. In some non-limiting examples, the subject has urethra dysfunction, and has at least one of increased oxidative stress in the urethra, increased mitochondrial dysfunction in the urethra, decreased urethra contractility, disrupted morphology of smooth or striated muscle or of mitochondria in the urethra, or decreased expression of α-SMA and cathepsin B in the urethra as compared with a subject without the urethra dysfunction. The method can improve one or more of these parameters.

In further embodiments, administering PNPase inhibitor or purine nucleoside substrate reduces ischemia, oxidative stress, or mitochondrial dysfunction in the bladder of a subject; increases bladder contractility; decreases urinary incontinence; increases bladder smooth muscle contractility; or decreases bladder wall volume. In additional embodiments, administering the PNPase inhibitor or purine nucleoside substrate reduces oxidative stress or mitochondrial dysfunction in the urethra of a subject; increases urethra contractility; decreases urinary incontinence; increases urethra smooth muscle contractility; improves morphology of smooth or striated muscle in the urethra; decreases disruption of mitochondria in the urethra; or increases expression of α-SMA and cathepsin B in the urethra.

Other methods are also disclosed, including methods of increasing bladder smooth muscle contractility or decreasing bladder wall volume in a subject (such as a human subject, such as a human at least 50 years old). These methods can include selecting a subject in need of increased bladder smooth muscle contractility or decreased bladder wall volume, and administering to the subject a therapeutically effective amount of a PNPase inhibitor or a PNPase purine nucleoside substrate. In some embodiments, the subject has bladder dysfunction or disease (such as at least one of increased void volume (such as pathological void volume), decreased void efficiency, decreased void frequency, increased bladder capacity, increased bladder storage, increased bladder wall volume, decreased sensitivity to stimuli (such as tactile stimuli, for example, abdominal or somatic stimuli), or cystitis (for example, interstitial cystitis, bladder pain syndrome, and/or radiation-induced cystitis). The subject can have increased bladder ischemia, increased oxidative stress in the bladder, or increased mitochondrial dysfunction in the bladder, as compared with a subject without the bladder dysfunction or disease.

Other methods disclosed herein also include methods of improving the morphology of the smooth or striated muscle in the urethra, decreasing disruption of mitochondria in the urethra, increasing expression of α-SMA and cathepsin B in the urethra, decreasing oxidative stress in the urethra, decreasing mitochondrial dysfunction in the urethra, or increasing urethra contractility, as compared with a subject without the urethra dysfunction, which can improve urethral function in the subject (such as in a human subject, such as a human at least 50 years old). These methods can include selecting a subject in need of improved morphology of the smooth or striated muscle in the urethra, decreased disruption of mitochondria in the urethra, increased expression of α-SMA and cathepsin B in the urethra, decreased oxidative stress in the urethra, decreased mitochondrial dysfunction in the urethra, or increased urethra contractility, and administering to the subject a therapeutically effective amount of a PNPase inhibitor or a PNPase purine nucleoside substrate. In some embodiments, the subject has urethra dysfunction (such as at least one of increased oxidative stress in the urethra, increased mitochondrial dysfunction in the urethra, decreased urethra contractility, disrupted morphology of smooth or striated muscle in the urethra, disrupted of mitochondria in the urethra; or decreased expression of α-SMA and cathepsin B in the urethra, as compared with a subject without the urethra dysfunction).

In any of the methods disclosed herein, the PNPase inhibitor or PNPase purine nucleoside substrate can include a guanine, guanosine, inosine, or hypoxanthine with a substituent (such as amine, hydroxyl, nitro, nitroso, alkoxy, carbonyl, halogen, carboxyl, ester, carbonate, amide, or haloaliphatic) at the 8-position; a transition state analog (such as forodesine, or a forodesine derivative, for example, DADMe-immucillin-H, DATMe-immucillin-H, or SerMe-immucillin-H, or a pharmaceutically acceptable salt thereof, for example, a chloride salt). In specific examples, the substituent is amine, such as 8-AG. In specific examples, the transition state analog is forodesine. Further, in any of the methods disclosed, the subject can have, for example, incontinence (such as stress, urgency, or spontaneous incontinence), overactive or underactive bladder, or urethra stricture. Further, in any of the examples herein, administering to any of the subjects herein a therapeutically effective amount of the PNPase inhibitor or a PNPase purine nucleoside substrate produces a decrease in at least one inflammation-associated cytokine (such as interleukin 1 beta (IL-1beta) or monocyte chemoattractant protein-1 (MCP-1)).

### III. Therapeutic Compounds and Pharmaceutical Compositions

It is disclosed herein that a PNPase inhibitor or a PNPase purine nucleoside substrate (for example, PNPase purine nucleoside substrates that can act as both a substrate or a PNPase inhibitor) can be used therapeutically. Examples of (PNPase) inhibitor or a PNPase purine nucleoside substrates that can be used therapeutically include guanine; guanosine; inosine; hypoxanthine; amiloride; an 8-substituted guanine (such as 8-AG, 8-hydroxyguanine, or 8-nitroguanine), guanosine (such as 8-aminoguanosine or 8-hydroxyguanosine), inosine (such as 8-aminoinosine), or hypoxanthine (such as 8-aminohypoxanthine); forodesine or a derivative thereof (for example, DADMe-Immucillin-H, DATMe-Immucillin-H, or SerMe-Immucillin-H, or such as described in U.S. Pat. Nos. 4,985,433; 4,985,434, 5,008,265; 5,008,270; 5,565,463, and 5,721,240 as well as US pat. pub. no. 2018/0258091A1, all of which are incorporated herein by reference in their entireties), or a pharmaceutically acceptable salt thereof (such as a chloride salt). The 8-substituted guanine, guanosine, inosine, and hypoxantine compounds are referred to as Formula 1 (guanine with a substituent at the 8 position), Formula 2 (guanosine with a substituent at the 8 position), Formula 3 (inosine with a substituent at the 8 position), and Formula 4 (hypoxantine with a substituent at the 8 position), respectively. The general chemical structures of these compounds are shown below and are further defined in the Terms section.

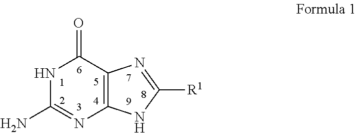

With reference to Formula 1, R¹ is selected from amine (—NR′R), hydroxyl (—OH), nitro (—NO₂), nitroso (—NO), alkoxy, carbonyl (C═O), halogen, carboxyl, ester, carbonate, amide, haloaliphatic, or hydrogen.

In some embodiments of Formula 1, R¹ is amine (—NR′R), and each of R and R′ independently are hydrogen, aliphatic, aryl, heteroaliphatic, or heteroaryl.

In some embodiments of Formula 1, R¹ is carbonyl (C═O), and the carbon located at the 8 position of Formula 1 forms a double bond with an oxygen atom.

In some embodiments of Formula 1, R¹ is halogen, and the halogen is bromo, fluoro, iodo, or chloro.

In some embodiments of Formula 1, R¹ is carboxyl (—C(O)OR), and R is hydrogen, aliphatic, aryl, heteroaliphatic, or heteroaryl.

In some embodiments of Formula 1, R¹ is ester (—OC(O)R), and R is hydrogen, aliphatic, aryl, heteroaliphatic, or heteroaryl.

In some embodiments of Formula 1, R¹ is carbonate (—OC(O)OR), and R is hydrogen, aliphatic, aryl, heteroaliphatic, or heteroaryl.

In some embodiments of Formula 1, R¹ is amide (—NC(O)R), and R is hydrogen, aliphatic, aryl, heteroaliphatic, or heteroaryl.

In some embodiments of Formula 1, R¹ is haloaliphatic (—CH₂X, —CHX₂, or —CX₃), and each X independently is halogen (Cl, Br, F, or I).

Any of the compound embodiments of Formula 1 can be included in a pharmaceutical composition and used in the methods disclosed herein.

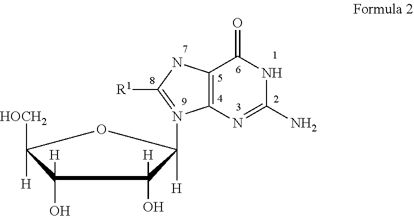

With reference to Formula 1, R¹ is selected from amine (—NR′R), hydroxyl (—OH), nitro (—NO₂), nitroso (—NO), alkoxy, carbonyl (C═O), halogen, carboxyl, ester, carbonate, amide, haloaliphatic, or hydrogen.

In some embodiments of Formula 2, R¹ is amine (—NR′R), and each of R and R′ independently are hydrogen, aliphatic, aryl, heteroaliphatic, or heteroaryl.

In some embodiments of Formula 2, R¹ is carbonyl (C═O), and the carbon located at the 8 position of Formula 1 forms a double bond with an oxygen atom.

In some embodiments of Formula 2, R¹ is halogen, and the halogen is bromo, fluoro, iodo, or chloro.

In some embodiments of Formula 2, R¹ is carboxyl (—C(O)OR), and R is hydrogen, aliphatic, aryl, heteroaliphatic, or heteroaryl.

In some embodiments of Formula 2, R¹ is ester (—OC(O)R), and R is hydrogen, aliphatic, aryl, heteroaliphatic, or heteroaryl.

In some embodiments of Formula 2, R¹ is carbonate (—OC(O)OR), and R is hydrogen, aliphatic, aryl, heteroaliphatic, or heteroaryl.

In some embodiments of Formula 2, R¹ is amide (—NC(O)R), and R is hydrogen, aliphatic, aryl, heteroaliphatic, r heteroaryl.

In some embodiments of Formula 2, R¹ is haloaliphatic (—CH₂X, —CHX₂, or —CX₃), and each X independently is halogen (Cl, Br, F, or I).

Any of the compound embodiments of Formula 2 can be included in a pharmaceutical composition and used in the methods disclosed herein.

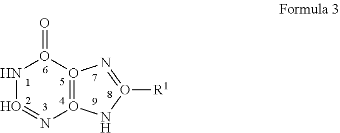

With reference to Formula 3, R¹ is selected from amine (—NR′R), hydroxyl (—OH), nitro (—NO₂), nitroso (—NO), alkoxy, carbonyl (C═O), halogen, carboxyl, ester, carbonate, amide, haloaliphatic, or hydrogen.

In some embodiments of Formula 3, R¹ is amine (—NR′R), and each of R and R′ independently are hydrogen, aliphatic, aryl, heteroaliphatic, or heteroaryl.

In some embodiments of Formula 3, R¹ is carbonyl (C═O), and the carbon located at the 8 position of Formula 1 forms a double bond with an oxygen atom.

In some embodiments of Formula 3, R¹ is halogen, and the halogen is bromo, fluoro, iodo, or chloro.

In some embodiments of Formula 3, R¹ is carboxyl (—C(O)OR), and R is hydrogen, aliphatic, aryl, heteroaliphatic, or heteroaryl.

In some embodiments of Formula 3, R¹ is ester (—OC(O)R), and R is hydrogen, aliphatic, aryl, heteroaliphatic, or heteroaryl.

In some embodiments of Formula 3, R¹ is carbonate (—OC(O)OR), and R is hydrogen, aliphatic, aryl, heteroaliphatic, or heteroaryl.

In some embodiments of Formula 3, R¹ is amide (—NC(O)R), and R is hydrogen, aliphatic, aryl, heteroaliphatic, or heteroaryl.

In some embodiments of Formula 3, R¹ is haloaliphatic (—CH₂X, —CHX₂, or —CX₃), and each X independently is halogen (Cl, Br, F, or I).

Any of the compound embodiments of Formula 3 can be included in a pharmaceutical composition and used in the methods disclosed herein.

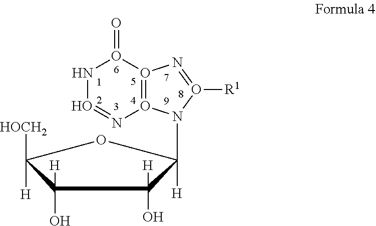

With reference to Formula 4, R¹ is selected from amine (—NR′R), hydroxyl (—OH), nitro (—NO₂), nitroso (—NO), alkoxy, carbonyl (C═O), halogen, carboxyl, ester, carbonate, amide, haloaliphatic, or hydrogen.

In some embodiments of Formula 4, R¹ is amine (—NR′R), and each of R and R′ independently are hydrogen, aliphatic, aryl, heteroaliphatic, or heteroaryl.

In some embodiments of Formula 4, R¹ is carbonyl (C═O), and the carbon located at the 8 position of Formula 1 forms a double bond with an oxygen atom.

In some embodiments of Formula 4, R¹ is halogen, and the halogen is bromo, fluoro, iodo, or chloro.

In some embodiments of Formula 4, R¹ is carboxyl (—C(O)OR), and R is hydrogen, aliphatic, aryl, heteroaliphatic, or heteroaryl.

In some embodiments of Formula 4, R¹ is ester (—OC(O)R), and R is hydrogen, aliphatic, aryl, heteroaliphatic, or heteroaryl.

In some embodiments of Formula 4, R¹ is carbonate (—OC(O)OR), and R is hydrogen, aliphatic, aryl, hetero aliphatic, or heteroaryl.

In some embodiments of Formula 4, R¹ is amide (—NC(O)R), and R is hydrogen, aliphatic, aryl, heteroaliphatic, or heteroaryl.

In some embodiments of Formula 4, R¹ is haloaliphatic (—CH₂X, —CHX₂, or —CX₃), and each X independently is halogen (Cl, Br, F, or I).

Any of the compound embodiments of Formula 4 can be included in a pharmaceutical composition and used in the methods disclosed herein.

Specific compounds of use in the methods disclosed herein include those shown in Table 2.

TABLE 2
Chemical structures for compounds of use in the methods disclosed herein

| Name | Structure | Name | Structure |
|---|---|---|---|
| Guanine | 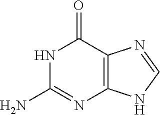 [SMILES: Nc1nc2[nH]cnc2c(=O)[nH]1] | Guanosine | 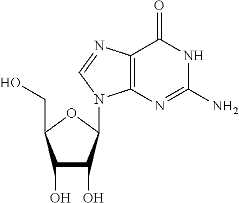 |
| 8- Aminoguanine | 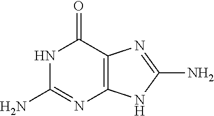 [SMILES: Nc1nc2[nH]c(N)nc2c(=O)[nH]1] | 8-Aminoguanosine | 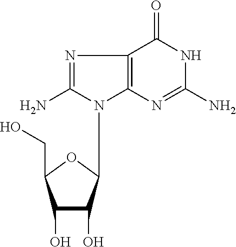 |
| 8- Hydroxyguanine | 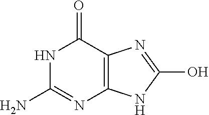 [SMILES: Nc1nc2[nH]c(O)nc2c(=O)[nH]1] | 8-Hydroxyguanosine | 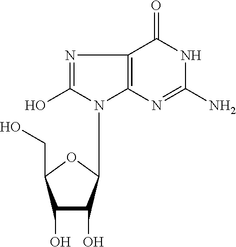 |
| 8- Nitroguanine | 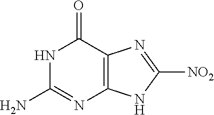 [SMILES: Nc1nc2[nH]c([N+](=O)[O-])nc2c(=O)[nH]1] | Amiloride | 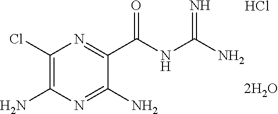 [SMILES: N=C(N)NC(=O)c1nc(Cl)c(N)nc1N ; Cl] |
| Inosine | 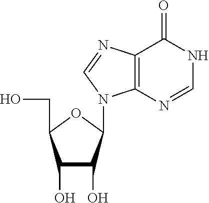 | Hypoxanthine | 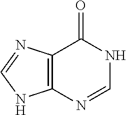 |
| 8- aminoinosine | 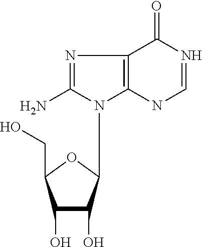 | 8-aminohypoxanthine | 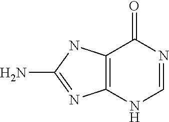 [SMILES: Nc1nc2[nH]cnc(=O)c2[nH]1] |

| | |
|---|---|
| Name(s) | Structure |
| Forodesine, Immucillin-H, and 7-[(2S,3S,4R,5R)-3,4-dihydroxy-5- (hydroxymethyl)pyrrolidin-2-yl)-3H,4H,5H-pyrrolo[3,2- d]pyrimidin-4-one | 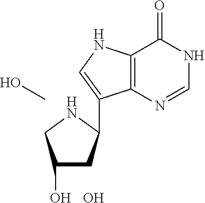 |
| DADMe-Immucillin-H, Ulodesine, and 7-(((3R,4R)-3-hydroxy-4- (hydroxymethyl)pyrrolidin-1-yl)methyl)-3H-pyrrolo[3,2- d]pyrimidin-4(5H)-one | 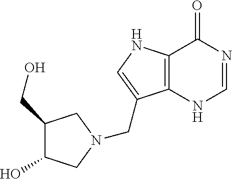 [SMILES: O=c1nc[nH]c2c(CN3C[C@H](CO)[C@@H](O)C3)c[nH]c12] |
| DATMe-Immucillin-H and 7-(((2R,3S)-1,3,4-trihydroxybutan-2- ylamino)methyl)-3H-pyrrolo[3,2-d]pyrimidin-4(5H)-one | 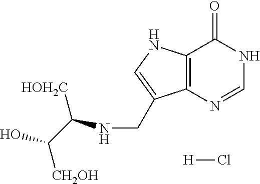 [SMILES: O=c1[nH]cnc2c(CN[C@H](CO)[C@@H](O)CO)c[nH]c12 ; Cl] |
| SerMe-Immucillin-H and 7-((1,3-dihydroxypropan-2- ylamino)methyl)-3H-pyrrolo[3,2-d]pyrimidin-4(5H)-one | 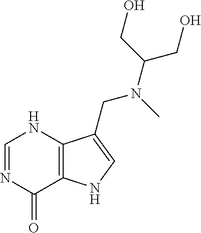 [SMILES: CN(Cc1c[nH]c2c(=O)nc[nH]c12)C(CO)CO] |

Compounds of use in the disclosed method include PNPase transition state analogs. As disclosed in U.S. Pat. No. 5,721,240, incorporated herein by reference in its entirety, 9-arylmethyl-substituted purines (including guanines) have been reported as PNP inhibitors in U.S. Pat. No. 4,772,606. PNPase inhibitory data cited in Drugs of the Future 13, 654 (1988) and Agents and Actions 21, 253 (1987) indicate that the 9-arylmethyl (Ar) substituted guanine derivatives of the formula:

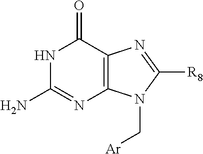

wherein R₈ represents hydrogen are markedly less potent PNP inhibitors than the corresponding compounds wherein R₈ represents amino (8-aminoguanines).

This U.S. patent disclosed inhibitors of the formula:

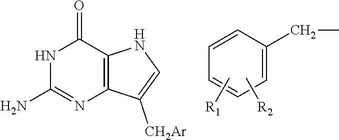

wherein CH₂Ar represents:

which R₁ represents hydrogen, halogen, C₁-C₃-alkyl, C₁-C₃-alkoxy, benzyloxy, hydroxy or trifluoromethyl; and R represents hydrogen, halogen, C₁-C₃-alkyl, C₁-C₃-alkoxy, benzyloxy, hydroxy or trifluoromethyl; provided that R₂ represents hydrogen or C₁-C₃-alkyl if R₁ represents trifluoromethyl, or that R₁ represents hydrogen or C₁-C₃-alkyl if R₂ represents trifluoromethyl; or

wherein CH₂Ar represents:

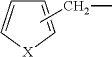

in which X represents sulfur or oxygen and in which attachment to the thiophene or furan ring is at the 2- or 3-position; and tautomers thereof.

This U.S. patent also discloses compounds specified by formulas II, III, and IV, which can also be used in the presently disclosed methods. Pharmaceutically acceptable salts and hydrates are also disclosed. In one embodiment, and pharmaceutically acceptable salt is a chloride salt. However, other salts can be utilized, such as alkali metal salts; esters such as acetate, butyrate, octinoate, palmitate, chlorobenzoates, benzoates, C₁-C₆ benzoates, succinates, and mesylate; salts of such esters; and nitrile oxides.

PCT Publication No. WO99/19338, incorporated herein by reference, discloses a compound genus as a new class of inhibitors of nucleoside metabolism, including forodesine, all of which can be used in the presently disclosed methods. PCT Publication No. WO 2016/110527, also incorporated herein by reference discloses methods for synthesis of forodesine. Forodesine, also known as immucillin-H and 7-[(2S,3S,4R,5R)-3,4-dihydroxy-5-(hydroxymethyl)-2-pyrrolidinyl]-1,5-dihydropyrrolo[2,3-e]pyrimidin-4-one, is an inhibitor of purine nucleoside phosphorylase.

In specific, non-limiting examples, the transition state analog can be forodesine (also known as immucillin-H and 7-[(2S,3S,4R,5R)-3,4-dihydroxy-5-(hydroxymethyl)pyrrolidin-2-yl]-3H,4H,5H-pyrrolo[3,2-d]pyrimidin-4-one) as well as derivatives thereof, such as DADMe-immucillin-H (also known as ulodesine and 7-(((3R,4R)-3-hydroxy-4-(hydroxymethyl)pyrrolidin-1-yl)methyl)-3H-pyrrolo[3,2-d]pyrimidin-4(5H)-one), DATMe-immucillin-H (also known as 7-(((2R,3S)-1,3,4-trihydroxybutan-2-ylamino)methyl)-3H-pyrrolo[3,2-d]pyrimidin-4(5H)-one), SerMe-immucillin-H (also known as 7-((1,3-dihydroxypropan-2-ylamino)methyl)-3H-pyrrolo[3,2-d]pyrimidin-4(5H)-one). Compounds of use are disclosed in U.S. Pat. Nos. 4,985,433; 4,985,434, 5,008,265; 5,008,270; 5,565,463, and 5,721,240 as well as U.S. Published Application No. 2018/0258091A1, all of which are incorporated herein by reference in their entireties. Pharmaceutically acceptable salts of these compounds are also of use, such as, but not limited to, chloride salts. Other salts can be utilized, such as alkali metal salts; esters such as acetate, butyrate, octinoate, palmitate, chlorobenzoates, benzoates, C₁-C₆ benzoates, succinates, and mesylate; salts of such esters; and nitrile oxides.

Any of these compounds can be included in pharmaceutical compositions and used in the methods disclosed herein.

### V. Pharmaceutical Compositions and Methods of Administration

Pharmaceutical compositions that include a PNPase inhibitor, such as, but not limited to, 8-substituted guanine, 8-substituted guanosine, 8-substituted inosine, or 8-substituted hypoxanthine (for example, 8-AG, 8-aminoguanosine, 8-hydroxyguanine, 8-hydroxyguanosine, 8-nitroguanine, 8-aminoinosine, or 8-aminohypoxanthine); amiloride; a PNPase transition state analog, such as forodesine or a forodesine derivative, for example, DADMe-immucillin-H, DATMe-immucillin-H, or SerMe-immucillin-H; a PNPase purine nucleoside substrate (such as guanosine and inosine, which can also act as a PNPase inhibitor); or a pharmaceutically acceptable salt thereof (such as a chloride salt, for example, a PNPase transition state analog chloride salt) can be formulated with an appropriate pharmaceutically acceptable carrier.

The dosage form of the pharmaceutical composition will be determined by the mode of administration chosen. For instance, in addition to injectable fluids, topical, inhalation, oral, infusion (such as to administered by catheter to the bladder or urethra) and suppository formulations can be employed. Inhalation preparations can be liquid (such as solutions or suspensions) and include mists, sprays, and the like. Oral formulations can be liquid (such as syrups, solutions, or suspensions) or solid (such as powders, pills, tablets, or capsules). Suppository preparations can also be solid, gel, or in a suspension form. Infusion preparations, administered by catheter, are generally administered as liquids. For solid compositions, conventional non-toxic solid carriers can include pharmaceutical grades of mannitol, lactose, cellulose, starch, or magnesium stearate. Actual methods of preparing such dosage forms are known, or will be apparent, to those skilled in the art.

The amount of PNPase inhibitor or PNPase purine nucleoside substrate administered will be dependent on the subject being treated, the severity of the affliction, and the manner of administration and is best left to the judgment of the prescribing clinician. Within these bounds, the formulation to be administered will contain a quantity of the active component(s) in amounts effective to achieve the desired effect in the subject being treated. A therapeutically effective amount of PNPase inhibitor or PNPase purine nucleoside substrate can be the amount of PNPase inhibitor or PNPase purine nucleoside substrate that is necessary to treat or lower the risk of a subject for a particular disease condition (see below).

The pharmaceutical compositions that include PNPase inhibitor (such as 8-substituted guanine, 8-substituted guanosine, 8-substituted inosine, 8-substituted hypoxanthine, amiloride, transition state analogs, or pharmaceutically acceptable salts thereof, for example, a transition state analog chloride salt) or PNPase purine nucleoside substrate (such as guanosine and inosine, which can also act as a PNPase inhibitor) can be formulated in unit dosage form, suitable for individual administration of precise dosages. A variety of dosages and dosing regimens are possible (for example, Kilpatrick et al., International Immunopharmacology, 3:541-548, 2003; Gandhi et al., Blood, 106(13):4253-4260, 2005, both of which are incorporated herein by reference in their entireties). In one specific, non-limiting example, a unit dosage (such as intravenous dosage) can contain about 1-50 μmoles/kg, such as about 1-5, 5-10, 10-20, 20-30, 30-40, or 40-50 μmoles/kg or about 33.5 μmoles/kg of a PNPase inhibitor or a PNPase purine nucleoside substrate. In other examples, a therapeutically effective amount of a PNPase inhibitor or a PNPase purine nucleoside substrate (such as oral dosage) is about 0.1-50 mg/kg, such as about 0.1-1, 1-5, 5-10, 5-20, 10-20, 20-30, 30-40, or 40-50 mg/kg or about 5, 10, 20, or 30 mg/kg (such as about 5-20 mg/kg/day). In further examples, a therapeutically effective amount of a PNPase inhibitor or a PNPase purine nucleoside substrate (such as an infusion dosage, for example, infusion into the bladder or urethra) is about 0.1-50 mg/kg, such as about 0.1-1, 1-5, 5-10, 10-20, 20-30, 30-40, or 40-50 mg/kg or about 0.9, 4.4, or 8.8 mg/kg or about 1-500 μM, such as about 1-5, 5-10, 10-100, or 100-500 μM, or about 5, 10, or 100 μM.

Pharmaceutically acceptable salts and hydrates are also disclosed. In one embodiment, and pharmaceutically acceptable salt is a chloride salt. However, other salts can be utilized, such as alkali metal salts; esters such as acetate, butyrate, octinoate, palmitate, chlorobenzoates, benzoates, C₁-C₆ benzoates, succinates, and mesylate; salts of such esters; and nitrile oxides.

A variety of treatment regimens are possible (for example, Kilpatrick et al., International Immunopharmacology, 3:541-548, 2003; Gandhi et al., Blood, 106(13):4253-4260, 2005, both of which are incorporated herein by reference in their entireties). Treatment with a therapeutically effective amount can be a single administration or multiple administrations. Treatment can involve daily or multi-daily or less than daily (such as weekly or monthly etc.) doses over a period of a few days to weeks or months, or even years. In a particular non-limiting example, treatment involves once daily dose or twice daily dose. The particular mode/manner of administration and the dosage regimen will be selected by the attending clinician, taking into account the particulars of the case (such as the subject, the disease, the disease state/severity involved, the particular treatment, and whether the treatment is prophylactic). In specific, non-limiting examples, administration can be oral, by infusion into the bladder or urethra (such as using a catheter), or by intravenous delivery.

The dosage form of the pharmaceutical composition will be determined by the mode of administration chosen. The pharmaceutically acceptable carriers and excipients useful in this disclosure are conventional (see, for example, Remington: The Science and Practice of Pharmacy, The University of the Sciences in Philadelphia, Editor, Lippincott, Williams, & Wilkins, Philadelphia, Pa., 21st Edition (2005)). For instance, parenteral formulations usually comprise injectable fluids that are pharmaceutically and physiologically acceptable fluid vehicles, such as water, physiological saline, other balanced salt solutions, aqueous dextrose, glycerol, or the like. In addition to injectable fluids, inhalational, and oral formulations can be employed. For solid compositions (such as powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, pH buffering agents, or the like, for example, sodium acetate or sorbitan monolaurate. Excipients that can be included are, for instance, proteins, such as human serum albumin or plasma preparations. Actual methods of preparing such dosage forms are known, or will be apparent, to those skilled in the art.

The compositions of this disclosure that include PNPase inhibitor (such as 8-substituted guanine, 8-substituted guanosine, 8-substituted inosine, 8-substituted hypoxanthine, amiloride, forodesine, or a forodesine derivative, for example, 8-AG, 8-aminoguanosine, 8-hydroxyguanine, 8-hydroxyguanosine, 8-nitroguanine, 8-aminoinosine, 8-aminohypoxanthine, DADMe-immucillin-H, DATMe-immucillin-H, or SerMe-immucillin-H) or PNPase purine nucleoside substrate (such as guanosine and inosine, which can also act as a PNPase inhibitor) can be administered to humans or other animals by any means, including orally, intravenously, intramuscularly, intraperitoneally (i.p.), intranasally, intradermally, intrathecally, subcutaneously, via catheter, via inhalation, or via suppository. In one non-limiting example, the composition is administered orally. In further examples, site-specific administration of the composition can be used, for example, by administering PNPase inhibitor or PNPase purine nucleoside substrate to the bladder directly, either by injection or infusion (such as using a catheter), or to the urethra directly, such as by injection. In yet another embodiment, such as for use with a PNPase transition state analog or pharmaceutically acceptable salt thereof (such as chloride salt), administration is intravenous or by infusion.

For oral administration, the pharmaceutical compositions can take the form of, for example, tablets or capsules prepared by conventional means with pharmaceutically acceptable excipients, such as binding agents (for example, pregelatinized maize starch, polyvinylpyrrolidone, or hydroxypropyl methylcellulose); fillers (for example, lactose, microcrystalline cellulose, or calcium hydrogen phosphate); lubricants (for example, magnesium stearate, talc, or silica); disintegrants (for example, potato starch or sodium starch glycolate); or wetting agents (for example, sodium lauryl sulfate). The tablets can be coated by methods well known in the art. Solid dosage forms for oral administration include, but are not limited to, capsules, tablets, pills, powders, and granules. In such solid dosage forms, the active compounds are mixed with at least one pharmaceutically acceptable excipient or carrier such as, but not limited to, sodium citrate or dicalcium phosphate. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives such as suspending agents (such as sorbitol syrup, cellulose derivatives, or hydrogenated edible fats); emulsifying agents (such as lecithin or acacia); non-aqueous vehicles (such as almond oil, oily esters, ethyl alcohol, or fractionated vegetable oils); and preservatives (such as methyl or propyl-p-hydroxybenzoates or sorbic acid). The preparations can also contain buffer salts, flavoring, coloring, and sweetening agents as appropriate. For solid compositions, conventional non-toxic solid carriers can include pharmaceutical grades of mannitol, lactose, starch, or magnesium stearate. Actual methods of preparing such dosage forms are known, or will be apparent, to those of ordinary skill in the art. Oral administration includes buccal or “sub-lingual” administration via membranes of the mouth. This can be accomplished using lozenges or a chewable gum.

Pharmaceutical compositions suitable for oral administration can be presented in discrete units each containing a predetermined amount of at least one therapeutic compound useful in the present methods; as a powder or granules; as a solution or a suspension in an aqueous or non-aqueous liquid; or as an oil-in-water or water-in-oil emulsion. As indicated, such compositions can be prepared by any suitable method of pharmacy, which includes the step of bringing into association the active compound(s) and the carrier (which can constitute one or more accessory ingredients). In general, the compositions are prepared by uniformly and intimately admixing the active compound with a liquid or finely divided solid carrier, or both, and then, if necessary, shaping the product.

For example, a tablet can be prepared by compressing or molding a powder or granules of the compound, optionally with one or more accessory ingredients. Compressed tablets can be prepared by compressing, in a suitable machine, the compound in a free-flowing form, such as a powder or granules optionally mixed with a binder, lubricant, inert diluent, or surface active/dispersing agent(s). Molded tablets can be made by molding, in a suitable machine, the powdered compound moistened with an inert liquid diluent.

Solid compositions of a similar type can also be employed as fillers in soft and hard filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They can optionally contain opacifying agents and can also be of a composition that they release the active ingredient(s) in a certain part of the intestinal tract, optionally, in a delayed manner. Examples of embedding compositions which can be used include polymeric substances and waxes. The active compounds can also be in micro-encapsulated form, if appropriate, with one or more of the above mentioned excipients.

Liquid dosage forms for oral administration include, but are not limited to, pharmaceutically acceptable emulsions, solutions, suspensions, syrups, teas, and elixirs. For administration to the bladder, the liquid dosage can be a pharmaceutically acceptable emulsion, solution, or suspension. In addition to the active compounds, the liquid dosage forms can contain inert diluents commonly used in the art such as, for example, water or other solvents, solubilizing agents, and emulsifiers, such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents. In some embodiments, suspensions, in addition to the active compounds, can contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

A drinkable tea can also be used in the present methods. A drinkable tea may be taken in a liquid form or in a once pulverized or granulated form together with water or hot water. When it is in a powdery or granular form, the drinkable tea may be contained in a cavity of mouth before taking hot water or water like the conventional powdery or granular drinkable tea, or it may be taken after once dissolving in hot water or water. One or more components, such as a sugar, mint, or other flavor, can be added to improve taste and easiness as a drinkable drug. Teas, syrups, and elixirs can be formulated with sweetening agents, for example glycerol, sorbitol, or sucrose. Such compositions can also contain a demulcent, a preservative, and flavoring and coloring agents.

Optionally, the pharmaceutical composition includes a parenteral carrier, and, in some embodiments, it is a solution that is isotonic with the blood of the recipient. Examples of such carrier vehicles include water, saline, Ringer's solution, and dextrose solution. Non-aqueous vehicles, such as fixed oils and ethyl oleate, are also useful herein, as well as liposomes.

The pharmaceutical compositions may be in the form of particles comprising a biodegradable polymer or a polysaccharide jellifying or bioadhesive polymer, an amphiphilic polymer, an agent modifying the interface properties of the particles and a pharmacologically active substance. These compositions exhibit certain biocompatibility features which allow a controlled release of the active substance. (See U.S. Pat. No. 5,700,486, incorporated herein by reference in its entirety).

In some embodiments, a PNPase inhibitor or a PNPase purine nucleoside substrate is included in a controlled release formulation, for example, a microencapsulated formulation. Various types of biodegradable and biocompatible polymers can be used, and methods of encapsulating a variety of synthetic compounds, proteins, and nucleic acids, have been well described in the art (see, for example, U.S. Patent Publication Nos. 2007/0148074; 2007/0092575; and 2006/0246139; U.S. Pat. Nos. 4,522,811; 5,753,234; and 7,081,489; PCT Publication No. WO/2006/052285; Benita, Microencapsulation: Methods and Industrial Applications, 2nd ed., CRC Press, 2006, all of which are incorporated by reference herein in their entireties).

In other embodiments, PNPase inhibitor or PNPase purine nucleoside substrate is included in a nanodispersion system. Nanodispersion systems and methods for producing such nanodispersions are well-known to one of skill in the art. (See, for example, U.S. Pat. No. 6,780,324; U.S. Pat. Publication No. 2009/0175953, both of which are incorporated herein by reference in their entireties). For example, a nanodispersion system includes a biologically active agent and a dispersing agent (such as a polymer, copolymer, or low molecular weight surfactant). Exemplary polymers or copolymers include polyvinylpyrrolidone (PVP), poly(D,L-lactic acid) (PLA), poly(D,L-lactic-co-glycolic acid (PLGA), poly(ethylene glycol). Exemplary low molecular weight surfactants include sodium dodecyl sulfate, hexadecyl pyridinium chloride, polysorbates, sorbitans, poly(oxyethylene) alkyl ethers, poly(oxyethylene) alkyl esters, and combinations thereof. In one example, the nanodispersion system includes PVP and PNPase inhibitor or PNPase purine nucleoside substrate (such as 80/20 w/w). In some examples, the nanodispersion is prepared using the solvent evaporation method (see, for example, Kanaze et al., Drug Dev. Indus. Pharm. 36:292-301, 2010; Kanaze et al., J. Appl. Polymer Sci. 102:460-471, 2006, both of which are incorporated herein by reference in their entireties).

Many types of release delivery systems are available and known to those of ordinary skill in the art. They include polymer based systems, such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109 (incorporated herein by reference in its entirety). Delivery systems also include non-polymer systems, such as lipids, including sterols such as cholesterol, cholesterol esters and fatty acids or neutral fats, such as mono-, di-, and tri-glycerides; hydrogel release systems; silastic systems; peptide-based systems; wax coatings; compressed tablets using conventional binders and excipients; partially fused implants; and the like. Specific examples include, but are not limited to, (a) erosional systems in which a PNPase inhibitor or a PNPase purine nucleoside substrate is contained in a form within a matrix, such as those described in U.S. Pat. Nos. 4,452,775; 4,667,014; 4,748,034; 5,239,660; and 6,218,371 (all of which are incorporated by reference herein in their entireties) and (b) diffusional systems in which an active component permeates at a controlled rate from a polymer, such as described in U.S. Pat. Nos. 3,832,253 and 3,854,480 (both of which are incorporated by reference in their entireties). In addition, pump-based hardware delivery systems can be used, some of which are adapted for implantation.

Use of a long-term sustained release implant may be particularly suitable for treatment of chronic conditions. Long-term release, as used herein, means that the implant is constructed and arranged to deliver therapeutic levels of the active ingredient for at least 30 days, and preferably 60 days. Long-term sustained release implants are well-known to those of ordinary skill in the art and include some of the release systems described above. These can be introduced, for example, subcutaneously or into the bladder.

### V. Methods of Treating Bladder and Urethra Dysfunction and Disease

Described herein are methods of increasing bladder smooth muscle contractility or decreasing bladder wall volume in a subject. Also described herein are methods of improving urethral function in a subject. The subject can be a human or veterinary subject. Veterinary subjects include domesticated animals or household pets, such as dogs, cats, horses, cows, and pigs. Non-human primates and wild animals can also be treated. In some examples, the methods include selecting a subject in need of increased bladder smooth muscle contractility or decreased bladder wall volume (such as a subject that has bladder dysfunction or disease). In some examples, the methods can include selecting a subject with urethral dysfunction or disease. The disease can be, for example, interstitial cystitis, bladder pain syndrome, and/or radiation induced cystitis.

In some examples, the methods include administering to the subject a therapeutically effective amount of a PNPase inhibitor or a PNPase purine nucleoside substrate, thereby increasing bladder smooth muscle contractility or increasing bladder wall volume in a subject. In some examples, the methods include administering to the subject a therapeutically effective amount of a PNPase inhibitor or a PNPase purine nucleoside substrate, thereby improving urethral function in the subject. In some examples, administration of PNPase inhibitor or a PNPase purine nucleoside substrate can improve morphology of smooth or striated muscle in the urethra, decrease disruption of mitochondria in the urethra, or increase expression of α-SMA and cathepsin B in the urethra.

Further described herein are methods of treating bladder or urethra dysfunction or disease. In some examples, the methods include selecting a subject with bladder or urethra dysfunction or disease. The subject can have interstitial cystitis, bladder pain syndrome, or radiation induced cystitis. The subject can be a human or veterinary subject. In some examples, the methods include administering to the subject a therapeutically effective amount of a PNPase inhibitor or a PNPase purine nucleoside substrate, thereby treating the bladder or urethra dysfunction or disease.

A variety of PNPase inhibitors or PNPase purine nucleoside substrates, such as those described herein, can be used in the methods. In some examples, a PNPase inhibitor is administered. In specific examples, a PNPase inhibitor includes guanine; guanosine; inosine; hypoxanthine; or guanine, guanosine, inosine, or hypoxanthine with a substituent at the 8-position, such as 8-substituted guanine, 8-substituted guanosine, 8-substituted inosine, or 8-substituted guanosine; a PNPase transition state analog (such as forodesine or a forodesine derivative, for example, DADMe-immucillin-H, DATMe-immucillin-H, or SerMe-immucillin-H); or a pharmaceutically acceptable salt thereof (such as a chloride salt, for example, a PNPase transition state analog chloride salt). Exemplary substituents (such as for 8-substituted guanine, 8-substituted guanosine, 8-substituted inosine, or 8-substituted guanosine) include amine, hydroxyl, nitro, nitroso, alkoxy, carbonyl, halogen, carboxyl, ester, carbonate, amide, or haloaliphatic. In specific examples, the substituent is amine. In specific, non-limiting examples, 8-substituted guanine, such as 8-AG can be used. In specific non-limiting examples, a PNPase transition state analog or a pharmaceutically acceptable salt thereof (such as a chloride salt) can be used. However, these are exemplary only. Any of the agents disclosed above are of use in these methods.

A variety of administration modes can be used. In some non-limiting examples, the PNPase inhibitor or PNPase purine nucleoside substrate is administered into the bladder or the urethra of the subject. In other non-limiting examples, the PNPase inhibitor or PNPase purine nucleoside substrate is administered orally to the subject. The PNPase inhibitor or PNPase purine nucleoside substrate can be administered intravenously, intramuscularly, intraperitoneally (i.p.), intranasally, intradermally, intrathecally, subcutaneously, via catheter, via inhalation, or via suppository.

In other examples, the PNPase inhibitor or a PNPase purine nucleoside substrate is administered to the subject once or more than once (such as repeatedly). In some examples, the PNPase inhibitor or PNPase purine nucleoside substrate is administered repeatedly, or one or more times (such as at least once, at least twice, at least three times, at least four times, at least five times, at least ten times, at least fifteen times, at least twenty times, at least thirty times, or more), such as one or more times daily, weekly, bimonthly, monthly, quarterly or per year. In other embodiments, the PNPase inhibitor or a PNPase purine nucleoside substrate is administered twice a day, daily, every other day, or 1, 2, 3, 4, 5, 6, or 7 times per week.

The subject (such as a human subject) can be any age, such as a child or an adult, for example, a younger adult, middle-aged adult, or older adult. For example, the subject can be at least about 1, at least about 2, at least about 5, at least about 10, at least about 12, at least about 16, at least about 18, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, or at least about 95, about 1-5, 5-10, 10-20, 20-30, 30-40, 40-50, 50-60, 60-70,70-80, 80-95, 1-20, 20-40, 40-60, 60-70, 70-80, 80-95, or at least about 50. The subject can be an older subject, and thus at least 50 years of age, such as at least 50, 55, 60, 65, or 70 years of age. In some examples, the bladder dysfunction or disease is a result of or correlates with age.

In some examples, the bladder or urethra dysfunction or disease includes voiding dysfunction. Voiding dysfunctions are common in the older adult (such as adults over 50 years old). Distinctive and reproducible changes occur in the detrusor ultrastructure in various forms or types of bladder dysfunction or disease, including overactivity and impaired contractility, which correlate with urodynamic behavior of the bladder over time (Elbadawi et al., J Urol 150: 1657, 1993; Resnick and Yalla, JAMA 257:3076-81, 1987; Strasser H et al., J Urol 2000; 164:1781-5, all of which are incorporated by reference in their entireties). Ultrastructural patterns of detrusor and corresponding urodynamic abnormalities in the aging bladder and geriatric voiding dysfunction were found to be reproducible and did not regress with time. These patterns correlate with detrusor overactivity, impaired detrusor contractile function (such as underactive bladder) with urinary retention, and urinary incontinence. Studies on cellular degeneration and ultrastructural change show a spectrum of age-related changes, including a decline in the smooth muscle to connective tissue ratio in males and females as well as swollen mitochondria that can be a predisposition to detrusor underactivity and other bladder dysfunction time (Elbadawi et al., J Urol 150: 1657, 1993; Resnick and Yalla. JAMA 257:3076-81, 1987; Strasser H et al. J Urol 2000; 164:1781-5; Elbadawi et al., J Urol 169, 540-546, 2003, all of which are incorporated by reference in their entireties). With increased age, apoptosis occurs and is associated with decreased striated muscle density (especially in women), which can result in decreases in urethral closure pressure and stress urinary incontinence (id., all of which are incorporated by reference in their entireties). Many of these changes observed with age in human bladder are consistent with the findings for aged rat bladders described herein. These include smooth muscle cell degeneration and distinctive ultrastructural impairment of the smooth muscle mitochondria.

In some examples, bladder or urethra dysfunction or disease includes inflammation. For example, inflammatory cytokine expression can be increased, and administration of a therapeutically effective amount of a PNPase inhibitor or a PNPase purine nucleoside substrate decreases expression of inflammatory cytokines. For example, the methods herein can includes measuring bladder inflammation, such as by measuring nucleic acid expression of inflammatory cytokines. For example, amplification of inflammatory cytokine nucleic acid molecules can be used. In specific, non-limiting examples, the threshold cycle (Ct) method can be used (see, e.g., Schmittgen and Livak, Nature Protocols, 3(6): 1101-1108, 2008, incorporated by reference herein in its entirety). Expression of a control gene, such as one or more housekeeping genes, can also be measured through nucleic acid expression (such as through amplification of β-actin nucleic acid molecules, for example, using the Ct method). In some examples, the at least one inflammatory cytokine includes interleukin 1 beta (IL-1beta) and/or monocyte chemoattractant protein-1 (MCP-1). For example, measuring inflammatory cytokine nucleic acid expression can include using one or both of the following sets of primers:

(forward; SEQ ID NO: 1)
5′-GGGATGATGACGACCTGCTA-3′
and
(reverse; SEQ ID NO: 2)
5′-TGTCGTTGCTTGTCTCTCCT-3′,
such as to measure IL-1beta;
(forward; SEQ ID NO: 3)
5′-TGCAGAGACACAGACAGAGG-3′
and
(reverse; SEQ ID NO: 4)
5′-GCCAGTGAATGAGTAGCAGC-3′,
such as to measure MCP-1.

In specific examples, a housekeeping gene can also be measured, such as β-actin, for example, using:

(forward; SEQ ID NO: 5)
5′-ACTCTTCCAGCCTTCCTTC-3′
and
(reverse; SEQ ID NO: 6)
5′-ATCTCCTTCTGCATCCTGTC-3′.

In specific, non-limiting examples, the methods include using amplification (such as by the Ct method) to measure expression of at least one inflammatory cytokine nucleic acid molecule using primers encoded by SEQ ID NOS: 1-2 and/or 3-4 and to measure expression of a control nucleic acid molecule (such as β-actin) in the subject using SEQ ID NOS: 5-6. In some examples, administration of a therapeutically effective amount of a PNPase inhibitor or a PNPase purine nucleoside substrate decreases the expression of IL-1beta and MCP-1 (such as measured using nucleic acid molecules, such as by Ct). For example, the expression of IL-1beta and MCP-1 can be decreased at least by 1-, 2-, 3-, 4-, 5-, 6-, 7-, 8-, 9-, 10-, 15-, or 20-fold, or about 1-2-fold, 1-5-fold, 1-10-fold, 1-20-fold, 5-10-fold, 5-15-fold, or 5-20-fold, or about 6- or 8-fold.
a. Bladder Dysfunction

In some embodiments, the subject has bladder dysfunction. In some examples, the subject has more than one signs or symptoms of bladder dysfunction, such as at least about two, at least about three, at least four, at least about five, at least about six, at least about seven, at least about eight, at least about nine, at least about ten, at least about eleven, at least about twelve, at least about thirteen, or at least about fourteen signs or symptoms.

In some examples, the bladder dysfunction includes increased void volume, decreased void efficiency (such as less effective emptying of the bladder; see, for example, Danziger and Grill, Am J Physiol Renal Physiol, 311: F459-F468, 2016, incorporated herein by reference in its entirety), decreased void frequency, increased bladder capacity, increased bladder storage, increased bladder wall volume, decreased sensitivity to stimuli (such as tactile stimuli, for example, abdominal or somatic stimuli), increased bladder ischemia, increased oxidative stress in the bladder, decreased bladder smooth muscle contractility, or increased mitochondrial dysfunction in the bladder, as compared with a subject without the bladder dysfunction. The disclosed methods can improve one or more of these parameters. In some embodiments, the subject has an underactive or an overactive bladder or incontinence. The disclosed method can reduce the incontinence.

In some embodiments, the subject is administered a pharmaceutical composition that include a PNPase inhibitor or a PNPase purine nucleoside substrate, such as 8-substituted guanine, guanosine, inosine, or hypoxanthine (such as 8-AG), a PNPase transition state analog (such as forodesine or a derivative thereof), or a pharmaceutically acceptable salt thereof (such as a PNPase transition state analog chloride salt) using any one of the pharmaceutical compositions and methods of administration described above. In some examples, administration of the pharmaceutical composition decreases the void volume, increases the void efficiency, increases the void frequency, decreases the bladder capacity, decreases the bladder storage, decreases the bladder wall volume, increases the sensitivity to stimuli (such as tactile stimuli, for example, abdominal or somatic stimuli), decreases the bladder ischemia, decreases the oxidative stress in the bladder, increases the bladder smooth muscle contractility, or decreases the mitochondrial dysfunction in the bladder, as compared with a subject without the bladder dysfunction for example, to at or near the levels of a control subject without bladder dysfunction. In some examples, administration of the pharmaceutical reduces or eliminates the underactive or an overactive bladder or incontinence. In specific, non-limiting examples, the pharmaceutical composition includes 8-AG or a PNPase transition state analog or pharmaceutically acceptable salt thereof (such as a PNPase transition state analog chloride salt).

In specific, non-limiting examples, the bladder dysfunction includes dysfunctional void volume, such as increased void volume compared with a control subject without bladder dysfunction. For example, the void volume can be increased by at least about 10%, at least about 20%, at least about 30%, at least about 40%, at least about 50%, at least about 75%, at least about 100%, at least about 150%, at least about 200%, at least about 250%, at least about 300%, at least about 400%, at least about 500%, or at least about 1000%, about 10-20%, 10-50%, 50-75%, 50-100%, 100-200%, 100-250%, 50-500%, 500-1000%, or about 150%. In some examples, the subject may be provided a pharmaceutical composition that includes PNPase inhibitor or PNPase purine nucleoside substrate, such as 8-substituted guanine, guanosine, inosine, or hypoxanthine or a PNPase transition state analog or pharmaceutically acceptable salt thereof (such as a PNPase transition state analog chloride salt), using any one of the pharmaceutical compositions and methods of administration described above. In some examples, administration of the pharmaceutical composition decreases the void volume, for example, to at or near the void volume of a control subject without bladder dysfunction. In specific, non-limiting examples, the pharmaceutical composition includes 8-AG or forodesine (such as forodesine hydrochloride).

In specific, non-limiting examples, the bladder dysfunction includes dysfunctional void frequency, such as decreased void frequency compared with a control subject without bladder dysfunction. For example, the void frequency can be decreased by at least about 5%, at least about 10%, at least about 15%, at least about 20%, at least about 25%, at least about 30%, at least about 35%, at least about 40%, at least about 50%, at least about 75%, at least about 100%, or at least about 250%, about 5-10%, 10-30%, 20-30%, 20-50%, 5-50%, 20-100%, 100-250%, or about 25%. In some examples, the subject may be provided a pharmaceutical composition that includes PNPase inhibitor or PNPase purine nucleoside substrate, such as 8-substituted guanine, guanosine, inosine, or hypoxanthine or a PNPase transition state analog or pharmaceutically acceptable salt thereof (such as a PNPase transition state analog chloride salt), using any one of the pharmaceutical compositions and methods of administration described above. In some examples, administration of the pharmaceutical composition increases the void frequency, for example, to at or near the void frequency of a control subject without bladder dysfunction. In specific, non-limiting examples, the pharmaceutical composition includes 8-AG or forodesine (such as forodesine hydrochloride).

In specific, non-limiting examples, the bladder dysfunction includes dysfunctional bladder wall volume, such as increased bladder wall volume compared with a control subject without bladder dysfunction. For example, the bladder wall volume can be increased by at least about 10%, at least about 20%, at least about 50%, at least about 75%, at least about 100%, at least about 150%, at least about 200%, at least about 250%, at least about 300%, at least about 450%, at least about 500%, or at least about 750%, or at least about 1000%, about 10-100%, 50-500%, 100-500%, 100-300%, 200-300%, or 100-1000%, or about 250%. In some examples, the subject may be provided a pharmaceutical composition that includes PNPase inhibitor or PNPase purine nucleoside substrate, such as 8-substituted guanine, guanosine, inosine, or hypoxanthine or a PNPase transition state analog or pharmaceutically acceptable salt thereof (such as a PNPase transition state analog chloride salt), using any one of the pharmaceutical compositions and methods of administration described above. In some examples, administration of the pharmaceutical composition decreases the bladder wall volume, for example, to at or near the bladder wall volume of a control subject without bladder dysfunction. In specific, non-limiting examples, the pharmaceutical composition includes 8-AG or forodesine (such as forodesine hydrochloride).

In specific, non-limiting examples, the bladder dysfunction includes dysfunctional bladder sensitivity to stimuli (such as, but not limited to, tactile stimuli, for example, abdominal or somatic stimuli), such as decreased sensitivity to stimuli compared with a control subject without bladder dysfunction. In some examples, electromyography is used to assess response to stimuli by bladder muscles and the urinary sphincter (such as the placement of sensors on the abdominal region or insertion of catheters into the urethra or rectum for measuring nerve impulses). In some examples, the sensitivity to stimuli of the subject with bladder dysfunction can be decreased by at least about 10%, at least about 20%, at least about 50%, at least about 75%, at least about 100%, at least about 150%, at least about 200%, at least about 250%, at least about 300%, at least about 450%, at least about 500%, or at least about 750%, or at least about 1000%, about 10-100%, 50-500%, 100-500%, 100-300%, 200-300%, or 100-1000%, or about 250%. In some examples, the subject may be provided a pharmaceutical composition that includes PNPase inhibitor or PNPase purine nucleoside substrate, such as 8-substituted guanine, guanosine, inosine, or hypoxanthine or a PNPase transition state analog or pharmaceutically acceptable salt thereof (such as a PNPase transition state analog chloride salt), using any one of the pharmaceutical compositions and methods of administration described above. In some examples, administration of the pharmaceutical composition increases the sensitivity to stimuli, for example, to at or near the sensitivity to stimuli of a control subject without bladder dysfunction. In specific, non-limiting examples, the pharmaceutical composition includes 8-AG or forodesine (such as forodesine hydrochloride).

In specific, non-limiting examples, the bladder dysfunction includes increased bladder ischemia, oxidative stress, or mitochondrial dysfunction, compared with a control subject without bladder dysfunction. Thus, the disclosed methods can decrease bladder dysfunction and, correspondingly, increase bladder function, such as by decreasing bladder ischemia, oxidative stress or mitochondrial dysfunction. Any method of detecting bladder ischemia, oxidative stress, or mitochondrial dysfunction can be used with the presently disclosed methods. In some examples, the bladder ischemia, oxidative stress, or mitochondrial dysfunction can be detected through structural changes in the bladder, such as structural changes to the smooth muscle cells or tissue or to the bladder vasculature. In some examples, such structural changes can be detected directly, such as using a biopsy procedure (see, for example, US Pat. Pub. Nos. 2005/0096263 and 2013/0102532, both of which are incorporated by reference in their entireties). For example, a biopsied sample of bladder smooth muscle tissue from a subject with bladder dysfunction can be used, for instance, to detect mitochondrial swelling or disruption in smooth muscle cells, compared with a biopsy sample from a control subject without bladder dysfunction. In some examples, a biopsied sample of vascular tissue from a subject with bladder dysfunction can be used, for instance, to detect ischemia in the bladder, such as by detecting tortuous vascular cells compared straight vascular cells, such as from a control subject without bladder dysfunction. In some examples, the subject may be provided a pharmaceutical composition that includes PNPase inhibitor or PNPase purine nucleoside substrate, such as 8-substituted guanine, guanosine, inosine, or hypoxanthine or a PNPase transition state analog or pharmaceutically acceptable salt thereof (such as a PNPase transition state analog chloride salt), using any one of the pharmaceutical compositions and methods of administration described above. In some examples, administration of the pharmaceutical composition decreases the bladder ischemia, oxidative stress, or mitochondrial dysfunction, to levels at or near a control subject without bladder dysfunction. In specific, non-limiting examples, the pharmaceutical composition includes 8-AG or forodesine (such as forodesine hydrochloride).

In specific, non-limiting examples, the bladder dysfunction includes underactive bladder (for example, difficulty with bladder emptying, such as hesitancy to start the stream, a poor or intermittent stream, or sensations of incomplete bladder emptying). The method can ease bladder emptying, reduce hesitancy start the stream, improve the urine stream, or reduce sensations of incomplete bladder emptying. Any method of diagnosing underactive bladder can be used with the presently disclosed methods. For example, underactive bladder can be diagnosed in various ways, including using a subject or patient voiding diary (to assess voided volumes and frequency of voiding), post-void residual volume, uninstrumented uroflow as well as a neurologic and pelvic examination, imaging for abnormal bladder morphology or vesicoureteral reflux/hydronephrosis, or invasive urodynamics. In some examples, the subject may be provided a pharmaceutical composition that includes PNPase inhibitor or PNPase purine nucleoside substrate, such as 8-substituted guanine, guanosine, inosine, or hypoxanthine or a PNPase transition state analog or pharmaceutically acceptable salt thereof (such as a PNPase transition state analog chloride salt), using any one of the pharmaceutical compositions and methods of administration described above. In some examples, administration of the pharmaceutical composition decreases or eliminates the underactive bladder. In specific, non-limiting examples, the pharmaceutical composition includes 8-AG or forodesine (such as forodesine hydrochloride).

In further embodiments, additional therapies or therapeutic agents can be used, administered, or included in the disclosed compositions, such as medications (such as bethanechol, doxazosin, and finasteride), double or triggered-reflex voiding, and intermittent self-catheterizations to drain the bladder.

In other embodiments, the bladder dysfunction includes overactive bladder (such as a frequent feeling of needing to urinate without or with a loss of bladder control, (‘urge incontinence’)). The disclosed methods can reduce the feeling of needing to urinate, or increase bladder control. Wet or dry overactive bladder can be treated using the methods disclosed herein. Overactive bladder can be diagnosed by the presence of characteristic symptoms, such as urgency), urinary frequency, nocturia, and urge incontinence), and can include use of a frequency/volume journal, cystourethroscopy, and questionnaires (such as general surveys of lower urinary tract symptoms and surveys specific to overactive bladder). In some examples, the subject may be provided a pharmaceutical composition that includes PNPase inhibitor or PNPase purine nucleoside substrate, such as 8-substituted guanine, guanosine, inosine, or hypoxanthine or a PNPase transition state analog or pharmaceutically acceptable salt thereof (such as a PNPase transition state analog chloride salt), using any one of the pharmaceutical compositions and methods of administration described above. In some examples, administration of the pharmaceutical composition decreases or eliminates the overactive bladder. In specific, non-limiting examples, the pharmaceutical composition includes 8-AG or forodesine (such as forodesine hydrochloride).

In some embodiments, additional therapies or therapeutic agents can be used, administered, or included in the disclosed compositions, such as pelvic floor exercises, bladder training, decreasing caffeine consumption, drinking moderate fluids, other behavioral methods, non-invasive electrical stimulation, botulinum toxin injection into the bladder, urinary catheter, or surgery. Medications of use with the disclosed methods, or that can be included in the pharmaceutical compositions, include anti-muscarinic agents, such as darifenacin, hyoscyamine, oxybutynin, tolterodine, solifenacin, trospium, and fesoterodine, as well as β3 adrenergic receptor agonists, such as mirabegron.

Additional treatments for bladder pain syndrome and interstitial cystitis can be used in some examples. For example, oral, intravesical, systemic, and mechanical treatments can be used, such as anti-nerve growth factors (NGF) treatment (such as Tanezumab or Fulranumab, monoclonal NGF neutralizing antibodies), blockade of tumor necrosis factor (TNF, for example adalimumab, a fully human high-affinity, recombinant immunoglobulin G1 anti-TNF monoclonal antibody), antagonists of Toll-Like receptors (TLRs, such as hydroxychloroquine, a TLR7 antagonist), modulators of immune/inflammatory processes (such as rosiptor or AQX-1125, which is an activator of SH2-containing inositol-5′-phosphatase 1 (SHIP1)), P2X3 receptor antagonists (such as AF-219), histamine antagonists, tricyclic agents (for polysynaptic effects), and cyclosporine A (for anti-T cell effect). In addition, a number of intravesical therapies have been used including dimethylsulfoxide (DMSO), heparin and lidocaine, pentosan polysulfate sodium (PPS, which is the only oral agent approved for BPS/IC by the FDA), sodium hyaluronate (HA), chondroitin sulfate (CS), sodium hyaluronate and chondroitin sulfate (HA-CS), oxychlorosene sodium (OS) and onabotulinum toxin A. The presently disclosed methods can be used in combination with any of these agents.

In specific, non-limiting examples, the bladder dysfunction includes decreased expression of α-SMA or microtubule-associated protein 1 light chain 3 alpha (LC3, such as the lipidated form) as compared with a subject without the bladder dysfunction, for example, to at or near the levels of a control subject without bladder dysfunction. Thus, the disclosed methods can decrease bladder dysfunction and, correspondingly, increase bladder function, such as by increasing expression of α-SMA or LC3 in the bladder. A variety of methods for detecting expression of α-SMA or LC3 in the bladder can be used with the presently disclosed methods, such as western blotting, immunohistochemistry, polymerase chain reaction (PCR; for example, using a microarray), and mass spectrometry (see, for example, Boland et al., Inflamm Bowel Dis., 21(2): 323-330, 2015; American Cancer Society, Tests used on biopsy and cytology specimens to diagnose cancer, 2015, accessed at cancer.org; Galamb et al., World J Gastroenterol., 12(43): 6998-7006, 2006; Gustashaw et al., Laboratory Medicine, 41(3):135-142, 2010, all of which are incorporated herein by reference in their entireties). In some examples, expression of α-SMA or LC3 in the bladder can be detected through measuring protein or mRNA levels in the bladder, such as in bladder tissue, for example, as obtaining using a biopsy procedure. In examples, a biopsied bladder sample from a subject with bladder dysfunction can be used, for instance, to detect expression of α-SMA or LC3, compared with a biopsy sample from a control subject without bladder dysfunction. In some examples, the subject may be provided a pharmaceutical composition that includes PNPase inhibitor or PNPase purine nucleoside substrate, such as 8-substituted guanine, guanosine, inosine, or hypoxanthine or a PNPase transition state analog or pharmaceutically acceptable salt thereof (such as a PNPase transition state analog chloride salt), using any one of the pharmaceutical compositions and methods of administration described above. In some examples, administration of the pharmaceutical composition increases the expression of LC3 or α-SMA. In specific, non-limiting examples, the pharmaceutical composition includes 8-AG or forodesine (such as forodesine hydrochloride).

In specific, non-limiting examples, the bladder dysfunction includes incontinence, such as stress, urgency, spontaneous, overflow, or mixed incontinence. In some examples, the incontinence is stress incontinence. In some examples, the incontinence is urgency incontinence. In some examples, the incontinence is spontaneous incontinence. In some examples, the incontinence is overflow incontinence. In some examples, the incontinence is mixed, such as more than one type of incontinence. These subjects can be selected for treatment, and any of these types of incontinence can be treated using the disclosed methods and pharmaceutical compositions.

Incontinence can be diagnosed in various ways, such as using a physical exam, which can include a bladder stressor, such as coughing; urinalysis, which can be used to detect infection, blood, or other abnormalities; bladder journal, which can include the subject or patient drinking, urination (including the amount of urine), urge to urinate, and incontinence; post-void residual measurements, such as voiding into a container that measures urine output with the remaining bladder urine measured using a catheter or ultrasound, for example, to detect an bladder obstruction or dysfunctional bladder nerves or muscles; urodynamic testing; or pelvic ultrasound.

In some examples, the subject may be provided a pharmaceutical composition that includes PNPase inhibitor or PNPase purine nucleoside substrate, such as 8-substituted guanine, guanosine, inosine, or hypoxanthine or a PNPase transition state analog or pharmaceutically acceptable salt thereof (such as a PNPase transition state analog chloride salt), using any one of the pharmaceutical compositions and methods of administration described above. In some examples, administration of the pharmaceutical composition decreases or eliminates the incontinence. In specific, non-limiting examples, the pharmaceutical composition includes 8-AG or forodesine (such as forodesine hydrochloride).

In further embodiments, therapies or therapeutic agents can be used, administered, or included in the disclosed compositions, such as bladder training (such as to delay urination), double voiding (such as to empty the bladder more completely and prevent overflow), scheduling urination, fluid and diet management (such as reducing or eliminating alcohol, caffeine, or acidic foods; reducing liquid consumption; losing weight; or increasing physical activity), pelvic floor muscle exercises, electrical stimulation, medications (such as anticholinergics, for example, oxybutynin (DITROPAN XL®), tolterodine (DETROL®), darifenacin (ENABLEX®), fesoterodine (TOVIAZ®), solifenacin (VESICARE®) and trospium (SANCTURA®); mirabegron (MYRBETRIQ®); alpha blockers, for example, tamsulosin (FLOMAX®), alfuzosin (UROXATRAL®), silodosin (RAPAFLO®), doxazosin (CARDURA®) and terazosin; or topical estrogen), devices (such as a urethral insert or a pessary), interventional therapies (such as bulking material injections, for example, botulinum toxin type A (BOTOX®) injections), nerve stimulators, surgery (such as sling pelvic procedures, bladder neck suspension, prolapse surgery, or artificial urinary sphincter), or absorbent pads and catheters.

b. Urethra Dysfunction

In some examples, the subject has at least one urethra dysfunction. In some examples, the subject has more than one sign or symptom of urethra dysfunction, such as at least about two, at least about three, at least four, or at least about five signs or symptoms. In some examples, the urethra dysfunction includes disordered morphology of smooth or striated muscle in the urethra, decreased expression of α-SMA or cathepsin B, increased oxidative stress in the urethra, decreased urethra smooth muscle contractility, or increased mitochondrial dysfunction or disruption in the urethra, as compared with a subject without the urethra dysfunction. The disclosed methods can improve one or more of these parameters. In some embodiments, the subject has urethral stricture or incontinence. The disclosed method can reduce the urethral stricture or incontinence.

The subject may be provided pharmaceutical compositions that include the PNPase inhibitor or a PNPase purine nucleoside substrate, such as 8-substituted guanine, guanosine, inosine, or hypoxanthine (such as 8-AG) or a PNPase transition state analog or pharmaceutically acceptable salt thereof (such as a PNPase transition state analog chloride salt), using any one of the pharmaceutical compositions and methods of administration described above. In some examples, administration of the pharmaceutical composition reduces disordered morphology of smooth or striated muscle in the urethra, increases expression of α-SMA or cathepsin B, decreases oxidative stress in the urethra, increases urethra smooth muscle contractility, or decreased mitochondrial dysfunction or disruption in the urethra, as compared with a subject without the bladder dysfunction for example, to at or near the levels of a control subject without bladder dysfunction. The disclosed methods can improve one or more of these parameters. In some examples, administration of the pharmaceutical reduces or eliminates urethral stricture or incontinence. In specific, non-limiting examples, the pharmaceutical composition includes 8-AG or forodesine (such as forodesine hydrochloride).

In specific, non-limiting examples, the urethra dysfunction includes increased oxidative stress, mitochondrial dysfunction, or disordered morphology of smooth or striated muscle in the urethra compared with a control subject without urethra dysfunction. Thus, the disclosed methods can decrease urethra dysfunction and, correspondingly, increase urethra function, such as by decreasing oxidative stress, mitochondrial dysfunction, or disordered morphology of smooth or striated muscle in the urethra. A variety of methods for detecting oxidative stress, mitochondrial dysfunction, or disordered morphology of smooth or striated muscle in the urethra can be used with the presently disclosed methods. In some examples, the urethra oxidative stress, mitochondrial dysfunction, or disordered morphology of smooth or striated muscle can be detected through structural changes in the urethra, such as structural changes to the smooth muscle cells or tissue. In some examples, such structural changes can be detected directly, such as using a biopsy procedure. For example, a biopsied sample of urethra smooth muscle tissue from a subject with urethra dysfunction can be used, for instance, to detect mitochondrial swelling or disruption in smooth muscle cells, compared with a biopsy sample from a control subject without urethra dysfunction. In some examples, the subject may be provided a pharmaceutical composition that includes PNPase inhibitor or PNPase purine nucleoside substrate, such as 8-substituted guanine, guanosine, inosine, or hypoxanthine or a PNPase transition state analog or pharmaceutically acceptable salt thereof (such as a PNPase transition state analog chloride salt), using any one of the pharmaceutical compositions and methods of administration described above. In some examples, administration of the pharmaceutical composition decreases the oxidative stress, mitochondrial dysfunction, or disordered morphology of smooth or striated muscle in the urethra to levels at or near a control subject without urethra dysfunction. In specific, non-limiting examples, the pharmaceutical composition includes 8-AG or forodesine (such as forodesine hydrochloride).

In specific, non-limiting examples, the urethra dysfunction includes decreased expression of alpha α-SMA or cathepsin B as compared with a subject without the bladder dysfunction, for example, to at or near the levels of a control subject without bladder dysfunction. Thus, the disclosed methods can decrease urethra dysfunction and, correspondingly, increase urethra function, such as by increasing expression of α-SMA or cathepsin B in the urethra. A variety of methods for detecting expression of α-SMA or cathepsin B in the urethra can be used with the presently disclosed methods, such as western blotting, immunohistochemistry, polymerase chain reaction (PCR; for example, using a microarray), and mass spectrometry (see, for example, Boland et al., Inflamm Bowel Dis., 21(2): 323-330, 2015; American Cancer Society, Tests used on biopsy and cytology specimens to diagnose cancer, 2015, accessed at cancer.org; Galamb et al., World J Gastroenterol., 12(43): 6998-7006, 2006; Gustashaw et al., Laboratory Medicine, 41(3):135-142, 2010, all of which are incorporated herein by reference in their entireties). In some examples, expression of α-SMA or cathepsin B in the urethra can be detected through measuring protein or mRNA levels in the urethra, such as in urethra tissue, for example, as obtaining using a biopsy procedure. In examples, a biopsied urethra sample from a subject with urethra dysfunction can be used, for instance, to detect expression of α-SMA or cathepsin B, compared with a biopsy sample from a control subject without urethra dysfunction. In some examples, the subject may be provided a pharmaceutical composition that includes PNPase inhibitor or PNPase purine nucleoside substrate, such as 8-substituted guanine, guanosine, inosine, or hypoxanthine or a PNPase transition state analog or pharmaceutically acceptable salt thereof (such as a PNPase transition state analog chloride salt), using any one of the pharmaceutical compositions and methods of administration described above. In some examples, administration of the pharmaceutical composition increases the expression of cathepsin B or α-SMA. In specific, non-limiting examples, the pharmaceutical composition includes 8-AG or forodesine (such as forodesine hydrochloride).

In specific, non-limiting examples, the urethra dysfunction includes incontinence, such as stress, spontaneous, overflow, or mixed incontinence. In some examples, the incontinence is stress incontinence. In some examples, the incontinence is spontaneous incontinence. In some examples, the incontinence is overflow incontinence. In some examples, the incontinence is mixed, such as more than one type of incontinence. These subjects can be selected for treatment, and any of these types of incontinence can be treated using the disclosed methods and pharmaceutical compositions. Incontinence can be diagnosed in various ways, such as using a physical exam, which can include a stress test; urinalysis, which can be used to detect infection, blood, or other abnormalities; journaling, which can include the subject or patient drinking, urination (including the amount of urine), urge to urinate, and incontinence; urodynamic testing; or cystoscopy.

In some examples, the subject may be provided a pharmaceutical composition that includes PNPase inhibitor or PNPase purine nucleoside substrate, such as 8-substituted guanine, guanosine, inosine, or hypoxanthine or a PNPase transition state analog or pharmaceutically acceptable salt thereof (such as a PNPase transition state analog chloride salt), using any one of the pharmaceutical compositions and methods of administration described above. In some examples, administration of the pharmaceutical composition decreases or eliminates the incontinence. In specific, non-limiting examples, the pharmaceutical composition includes 8-AG or forodesine (such as forodesine hydrochloride).

In further embodiments, therapies or therapeutic agents can be used, administered, or included in the disclosed compositions. For example, therapies for exertion and physical activity related urinary loss (stress urinary incontinence, SUI), bladder storage overactivity related urinary loss (urge urinary incontinence, UUI), mixed urinary incontinence (combination of SUI and UUI-MUI), and insensate urinary loss (loss of urine without any prodrome, which is common in older patients) are useful herein. Other therapies for use in combination with the methods disclosed herein include bladder training (such as to delay urination), double voiding (such as to empty the bladder more completely and prevent overflow), scheduling urination, fluid and diet management (such as reducing or eliminating alcohol, caffeine, or acidic foods; reducing liquid consumption; losing weight; or increasing physical activity), pelvic floor muscle exercises, electrical stimulation, medications (such as anticholinergics, for example, oxybutynin (DITROPAN XL®), tolterodine (DETROL®), darifenacin (ENABLEX®), fesoterodine (TOVIAZ®), solifenacin (VESICARE®) and trospium (SANCTURA®); mirabegron (MYRBETRIQ®); alpha blockers, for example, tamsulosin (FLOMAX®), alfuzosin (UROXATRAL®), silodosin (RAPAFLO®), doxazosin (CARDURA®) and terazosin; or topical estrogen), devices (such as a urethral insert or a pessary), interventional therapies (such as bulking material injections, for example, botulinum toxin type A (BOTOX®) injections), nerve stimulators, surgery (such as sling pelvic procedures, bladder neck suspension, prolapse surgery, or artificial urinary sphincter), absorbent pads and catheters, or serotonin/norepinephrine reuptake inhibitors (such as with SUI).

c. Bladder Disease

In some embodiments, the subject has bladder disease (such as interstitial cystitis, bladder pain syndrome, and/or radiation-induced cystitis). In some examples, the subject has more than one signs or symptoms of bladder disease, such as at least about two, at least about three, or at least four signs or symptoms of bladder disease. In some examples, the bladder disease includes voiding dysfunction, bladder inflammation, bladder hypernemia, and increased pain. The disclosed methods can improve one or more of these parameters. In some embodiments, the subject has an cystitis. The disclosed method can reduce the cystitis.

In some embodiments, the subject is administered a pharmaceutical composition that include a PNPase inhibitor or a PNPase purine nucleoside substrate, such as 8-substituted guanine, guanosine, inosine, or hypoxanthine (such as 8-AG), a PNPase transition state analog (such as forodesine or a derivative thereof), or a pharmaceutically acceptable salt thereof (such as a PNPase transition state analog chloride salt) using any one of the pharmaceutical compositions and methods of administration described above. In some examples, administration of the pharmaceutical composition decreases the voiding dysfunction, bladder inflammation, bladder hypernemia, and pain. In some examples, administration of the pharmaceutical reduces or eliminates the cystitis. In specific, non-limiting examples, the pharmaceutical composition includes 8-AG or a PNPase transition state analog or pharmaceutically acceptable salt thereof (such as a PNPase transition state analog chloride salt).

In specific, non-limiting examples, the bladder disease includes dysfunctional void frequency, such as decreased void frequency compared with a control subject without bladder disease. For example, the void frequency can be decreased by at least about 5%, at least about 10%, at least about 15%, at least about 20%, at least about 25%, at least about 30%, at least about 35%, at least about 40%, at least about 50%, at least about 75%, at least about 100%, or at least about 250%, about 5-10%, 10-30%, 20-30%, 20-50%, 5-50%, 20-100%, 40-80%, 50-75%, 100-250%, or about 50% or 75%. In some examples, the subject may be provided a pharmaceutical composition that includes PNPase inhibitor or PNPase purine nucleoside substrate, such as 8-substituted guanine, guanosine, inosine, or hypoxanthine or a PNPase transition state analog or pharmaceutically acceptable salt thereof (such as a PNPase transition state analog chloride salt), using any one of the pharmaceutical compositions and methods of administration described above. In some examples, administration of the pharmaceutical composition increases the void frequency, for example, to at or near the void frequency of a control subject without bladder disease. In specific, non-limiting examples, the pharmaceutical composition includes 8-AG.

In specific, non-limiting examples, the bladder disease includes increased bladder pain or decreased pain threshold, such as increased bladder pain or decreased pain threshold compared with a control subject without bladder disease. In some examples, the bladder pain of the subject with bladder disease can be decreased by at least about 10%, at least about 20%, at least about 40%, at least about 50%, at least about 60%, at least about 75%, at least about 80%, at least about 90%, at least about 100%, at least about 150%, at least about 200%, at least about 250%, at least about 300%, at least about 450%, at least about 500%, or at least about 750%, or at least about 1000%, about 10-100%, 50-500%, 60-90%, 70-90%, 80-90%, 100-500%, 100-300%, 200-300%, or 100-1000%, or about 80%. In some examples, the subject may be provided a pharmaceutical composition that includes PNPase inhibitor or PNPase purine nucleoside substrate, such as 8-substituted guanine, guanosine, inosine, or hypoxanthine or a PNPase transition state analog or pharmaceutically acceptable salt thereof (such as a PNPase transition state analog chloride salt), using any one of the pharmaceutical compositions and methods of administration described above. In some examples, administration of the pharmaceutical composition decreases the bladder pain, for example, to at or near the pain level of a control subject without bladder disease. In specific, non-limiting examples, the pharmaceutical composition includes 8-AG.

In specific, non-limiting examples, the bladder disease includes increased bladder inflammation, compared with a control subject without bladder disease. Thus, the disclosed methods can decrease bladder disease and, correspondingly, decrease bladder inflammation. Any method of detecting bladder inflammation can be used with the presently disclosed methods. In some examples, the bladder inflammation can be detected through expression changes of inflammatory cytokines in the bladder, such as interleukin 1 beta (IL-1beta) or monocyte chemoattractant protein-1 (MCP-1). In some examples, such expression changes can be detected by measuring nucleic acid expression of inflammatory cytokines. For example, amplification of inflammatory cytokine nucleic acid molecules can be used. In specific, non-limiting examples, the threshold cycle (Ct) method can be used (see, e.g., Schmittgen and Livak, Nature Protocols, 3(6): 1101-1108, 2008, incorporated by reference herein in its entirety). Expression of a control gene, such as one or more housekeeping genes, can also be measured through nucleic acid expression (such as through amplification of β-actin nucleic acid molecules, for example, using the Ct method). In some examples, the at least one inflammatory cytokine includes interleukin 1 beta (IL-1beta) and/or monocyte chemoattractant protein-1 (MCP-1). For example, measuring inflammatory cytokine nucleic acid expression can include using one or both of the following sets of primers:

(forward; SEQ ID NO: 1)
5′-GGGATGATGACGACCTGCTA-3′
and
(reverse; SEQ ID NO: 2)
5′-TGTCGTTGCTTGTCTCTCCT-3′,
such as to measure IL-1beta;
(forward; SEQ ID NO: 3)
5′-TGCAGAGACACAGACAGAGG-3′
and
(reverse; SEQ ID NO: 4)
5′-GCCAGTGAATGAGTAGCAGC-3′,
such as to measure MCP-1.

In specific examples, a housekeeping gene can also be measured, such as β-actin, for example, using:

(forward; SEQ ID NO: 5)
5′-ACTCTTCCAGCCTTCCTTC-3′
and
(reverse; SEQ ID NO: 6)
5′-ATCTCCTTCTGCATCCTGTC-3′.

In specific, non-limiting examples, the methods include using amplification (such as by the Ct method) to measure expression of at least one inflammatory cytokine nucleic acid molecule using primers encoded by SEQ ID NOS: 1-2 and/or 3-4 and to measure expression of a control nucleic acid molecule (such as β-actin) in the subject using SEQ ID NOS: 5-6. In some examples, administration of a therapeutically effective amount of a PNPase inhibitor or a PNPase purine nucleoside substrate decreases the expression of IL-1beta and MCP-1 (such as measured using nucleic acid molecules, such as by Ct). For example, the expression of IL-1beta and MCP-1 can be decreased at least by 1-, 2-, 3-, 4-, 5-, 6-, 7-, 8-, 9-, 10-, 15-, or 20-fold, or about 1-2-fold, 1-5-fold, 1-10-fold, 1-20-fold, 5-10-fold, 5-15-fold, or 5-20-fold, or about 6- or 8-fold. In some examples, the subject may be provided a pharmaceutical composition that includes PNPase inhibitor or PNPase purine nucleoside substrate, such as 8-substituted guanine, guanosine, inosine, or hypoxanthine or a PNPase transition state analog or pharmaceutically acceptable salt thereof (such as a PNPase transition state analog chloride salt), using any one of the pharmaceutical compositions and methods of administration described above. In some examples, administration of the pharmaceutical composition decreases the bladder inflammation to levels at or near a control subject without bladder disease. In specific, non-limiting examples, the pharmaceutical composition includes 8-AG.

In further embodiments, additional therapies or therapeutic agents can be used, administered, or included in the disclosed compositions, such as bladder training (such as to delay urination), double voiding (such as to empty the bladder more completely and prevent overflow), scheduling urination, fluid and diet management (such as reducing or eliminating alcohol, caffeine, or acidic foods; reducing liquid consumption; losing weight; or increasing physical activity), pelvic floor muscle exercises, electrical stimulation, medications (such as anticholinergics, for example, oxybutynin (DITROPAN XL®), tolterodine (DETROL®), darifenacin (ENABLEX®), fesoterodine (TOVIAZ®), solifenacin (VESICARE®) and trospium (SANCTURA®); mirabegron (MYRBETRIQ®); alpha blockers, for example, tamsulosin (FLOMAX®), alfuzosin (UROXATRAL®), silodosin (RAPAFLO®), doxazosin (CARDURA®) and terazosin; or topical estrogen), devices (such as a urethral insert or a pessary), interventional therapies (such as bulking material injections, for example, botulinum toxin type A (BOTOX®) injections), nerve stimulators, surgery (such as sling pelvic procedures, bladder neck suspension, prolapse surgery, or artificial urinary sphincter), or absorbent pads and catheters. Additional treatments for bladder pain syndrome and interstitial cystitis can be used in some examples. For example, oral, intravesical, systemic, and mechanical treatments can be used, such as anti-nerve growth factors (NGF) treatment (such as Tanezumab or Fulranumab, monoclonal NGF neutralizing antibodies), blockade of tumor necrosis factor (TNF, for example adalimumab, a fully human high-affinity, recombinant immunoglobulin G1 anti-TNF monoclonal antibody), antagonists of Toll-Like receptors (TLRs, such as hydroxychloroquine, a TLR7 antagonist), modulators of immune/inflammatory processes (such as rosiptor or AQX-1125, which is an activator of SH2-containing inositol-5′-phosphatase 1 (SHIP1)), P2X3 receptor antagonists (such as AF-219), histamine antagonists, tricyclic agents (for polysynaptic effects), and cyclosporine A (for anti-T cell effect). In addition, a number of intravesical therapies have been used including dimethylsulfoxide (DMSO), heparin and lidocaine, pentosan polysulfate sodium (PPS, which is the only oral agent approved for BPS/IC by the FDA), sodium hyaluronate (HA), chondroitin sulfate (CS), sodium hyaluronate and chondroitin sulfate (HA-CS), oxychlorosene sodium (OS) and onabotulinum toxin A. The presently disclosed methods can be used in combination with any of these agents.

### EXAMPLES

8-substituted guanine or 8-substituted guanosine compounds are used in treating bladder dysfunction or disease, such as in aging, by reversing and restoring the bladder to a younger state. The following examples are provided to illustrate particular features of certain embodiments, but the scope of the claims should not be limited to those features exemplified. The disclosure herein can slow the negative effects of aging and frailty (such as ischemia, oxidative stress, and mitochondrial dysfunction) on cellular structures within the bladder wall (such as urothelial, neural and smooth muscle). Geriatric-associated lower urinary tract dysfunctions associated with above conditions include: poor bladder contraction (such as the underactive bladder syndrome, UAB), overactive bladder (often coexistent with UAB), and urinary incontinence (such as stress, urgency, and spontaneous), which can arise from one or a combination of bladder and urethral dysfunctions (Wein and Moy. Campbell-Walsh Urology, 2007, p. 2011-44; Andersson et al. Heath Publications Ltd, 2009, p 631-99; Abrams et al., Campbell-Walsh Urology, 2007, p. 2079-90; Abrams et al., Neurourol Urodyn 2002, 21:167-78; Osman et al. Eur Urol 2014, 65:389-98; Van Koeveringe and Rademakers, Minerva Urol Nephol. 2015, 67:139-48; Smith et al., Neurourol Urodyn 2016: 35:312-7; Chapple et al., Neurourol Urodyn 2018 37:2928-2931; Abrams et al. Neurorology Urodyn 2002; 21:167-78; Andersson K E. Curr Opin Urol 2014, 24:363-9; Klauser et al. J Ultrasound Med 2004, 23:63107; Strasser et al. J Urol 2000, 164:1781-5; Osman et al. In Campbell-Walsh Urology, 2015; Guzzo et al., Primer of Geriatric Urology, 2016, all of which are incorporated by reference in their entireties). With treatment, symptomatic improvement or resolution of these conditions can occur. Additionally, the ability to treat individuals at risk for these conditions as a prophylactic to aging-induced pathologies (such as using the methods disclosed herein) can provide early intervention capabilities and avoidance of chronic changes associated with the above morbidities (such as frailty and aging).

Administration of 8-aminoguanosine and 8-AG can also induce diuresis, natriuresis, and glucosuria, but reduce potassium excretion (Jackson et al., J Pharmacol Exp Ther, 359:420-435, 30, 2016, incorporated herein by reference in its entirety). Further, chronic oral treatment of rats with 8-aminoguanosine and 8-AG attenuate the development of deoxycorticosterone/salt-induced hypertension (Jackson et al., J Pharmacol Exp Ther, 359:420-435, 2016, incorporated herein by reference in its entirety). In addition, 8-aminoguanosine exerts diuretic, natriuretic, and glucosuric activity via conversion to 8-AG, but has direct antikaliuretic effects (Jackson et al., J Pharmacol Exp Ther, 363:358-366, 2017, incorporated herein by reference in its entirety). Moreover, 8-AG induces diuresis, natriuresis, and glucosuria by inhibiting PNPase and reduces potassium excretion by inhibiting Rac 1 (Jackson et al., J Am Heart Assoc, in press, 2018). 8-AG, 8-aminoguanosine, and other PNPase inhibitors or substrates also have additional benefits for sickle cell disease, pulmonary hypertension, and stroke (Jackson and Tofovic, Methods for Treatment Using Small Molecule Potassium-Sparing Diuretics and Natriuretics, International Publication Number WO 2018/045045 A1, incorporated herein by reference in its entirety). Described herein, forodesine mimics the effects of 8-AG. Further, PNPase inhibition blocks metabolism of inosine to hypoxanthine and guanosine to guanine, uro-protective effects of PNPase inhibitors (such as 8-AG), and inosine and guanosine are considered uro-protective purines, while hypoxanthine is considered a uro-damaging purine.

### Example 1

Experimental design: Young (3 months of age) and aged (25 to 30 months of age) Fisher 344 rats were used in these experiments. Some of the aged Fisher 344 rats were treated with 8-AG (5 mg kg/day via drinking water) for 1 month.

Voiding analysis: Rats were placed in metabolic cages for 24 hr (once per week for the duration of the study). Voided urine was collected in cups attached to force displacement transducers (GRASS TECHNOLOGIES®, Warwick, R.I.) connected to a computer running WINDAQ™ data acquisition software (DATAQ™ Instruments Inc., Akron, Ohio). Data were averaged for 24 h, and voiding frequency, total voided volume, and volume per void were analyzed. Voiding frequency was calculated as the number of voiding events per hour during 24 h, and volume per void, which defines bladder capacity, was calculated as an average of the voids occurring during these periods.

Von Frey testing: Forty-eight hours after metabolism studies, tactile sensitivity was measured (once per week for the duration of the study) using von Frey nylon filaments applied to the suprapubic and plantar hindpaw (Stoelting Co., Wood Dale, Ill., USA). The median 50% withdrawal threshold (using up-down method) with positive responses are recorded as sharp retraction, licking/scratching, or vocalization.

Bladder volume measurements: Changes in bladder wall thickness in all groups (young, aged, and aged+treatment) were measured by placing displacement markers on the bladder surface (in situ) while volume measurements were recorded. Custom computer algorithms were used to calculate longitudinal, circumferential, and area strain from measurements of surface marker positions.

Western immunoblotting and transmission electron microscopy: Following these testing paradigms, rats were deeply anesthetized, bladders were removed, and one half of each bladder (using the smooth muscle layer) was homogenized using Lysing Matrix D in a FASTPREP-24™ instrument (MP BIOMEDICALS™, Solon, Ohio) in HBSS (5 mM KCl, 0.3 mM KH₂PO₄, 138 mM NaCl, 4 mM NaHO₃, 0.3 mM Na₂HCO₃, 0.3 mM Na₂HPO₄, 5.6 mM glucose and 10 mM Hepes, pH 7.4) containing complete protease inhibitor cocktail (1 tablet/10 ml, ROCHE™, Indianapolis, Ind.) and phosphatase inhibitor cocktail (SIGMA®, 1:100). Whole rat kidney was also homogenized using the same procedure. After centrifugation (13000 rpm; 15 min at 4° C.), the membrane protein fraction was prepared by suspending the membrane pellets in lysis buffer containing 0.3 M NaCl, 50 mM Tris-HCl (pH 7.6), 0.5% Triton X-100, and the same concentration of protease inhibitors as above. The suspensions were incubated on ice and centrifuged (13000 rpm; 15 min at 4° C.). The protein concentrations of the combined supernatants were determined using the PIERCE™ BCA protein assay (THERMO SCIENTIFIC™, Rockford, Ill.). After denaturation (100° C. for 5 min) in the presence of Laemmli sample buffer, lysate from each sample was separated on a 4-15% TGX STAIN-FREE™ SDS-PAGE gel (BIO-RAD™ Laboratories, Hercules, Calif.). As a loading control, total protein per sample was determined using BIO-RAD™ Stain Free SDS-PAGE gel technology. UV-activated protein fluorescence was imaged on a CHEMIDOC™ MP (BIO-RAD™). After proteins were transferred to polyvinylidene fluoride membranes, the membranes were incubated in 5% (w/v) dried milk dissolved in TBS-T (20 mM Trizma, 137 mM NaCl, 0.1% Tween-20, pH 7.6), rinsed with TBS-T, and incubated overnight at 4° C. with primary antibody (p16, ABCAM® or AVP V1a or V2, Alpha Diagnostics) diluted in TBS-T containing 5% (w/v) milk. After washing in TBS-T, the membranes were incubated with secondary antibody (either donkey anti-rabbit or sheep anti-mouse; 1:5000 GE™ AMERSHAM™ Pittsburgh Pa.) for 1 hour in 5% (w/v) milk TBS-T, washed, incubated in WESTERNBRIGHT™ QUANTUM™ (ADVANSTA™, Menlo Park, Calif.), and then imaged on a CHEMIDOC™ MP (BIO-RAD™). The volume (intensity) of each protein species was determined and normalized to total protein using Image Lab software (BIO-RAD™).

Transmission electron microscopy (TEM): The other half of each urinary bladder from each group (young, aged, and aged+treatment) were fixed in cold 2.5% glutaraldehyde in 0.01 M PBS. The specimens were rinsed in PBS, post-fixed in 1% osmium tetroxide with 1% potassium ferricyanide, rinsed in PBS, dehydrated through a graded series of ethanol and propylene oxide, and embedded in POLY/BED® 812 (Luft formulations). Semi-thin (300 nm) sections were cut on a LEICA® Reichart Ultracut, stained with 0.5% toluidine blue in 1% sodium borate and examined under the light microscope. Ultrathin sections (65 nm) were stained with uranyl acetate and Reynold's lead citrate and examined on JEOL 1400 transmission electron microscope with a side mount AMT 2k digital camera (Advanced Microscopy Techniques, Danvers, Mass.).

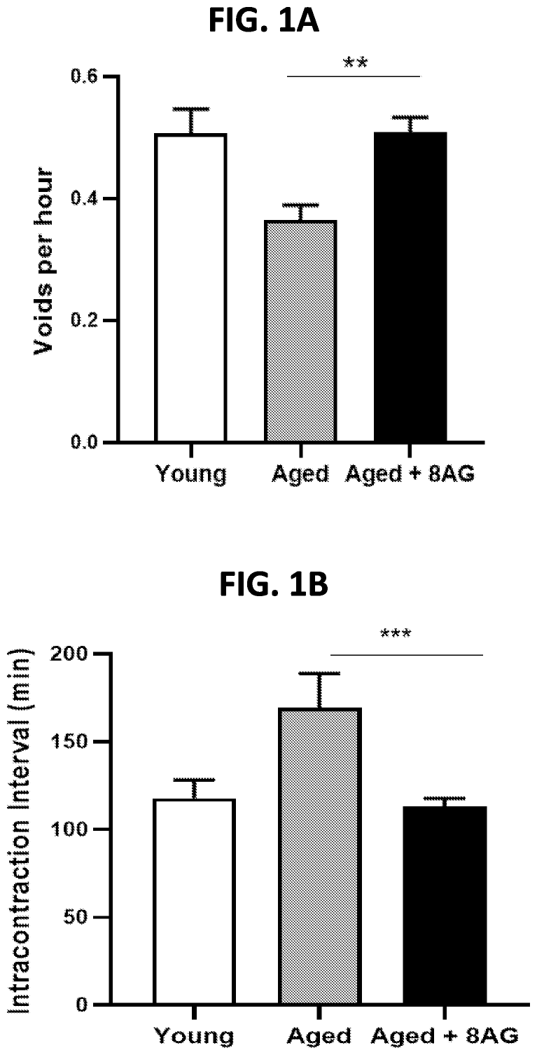

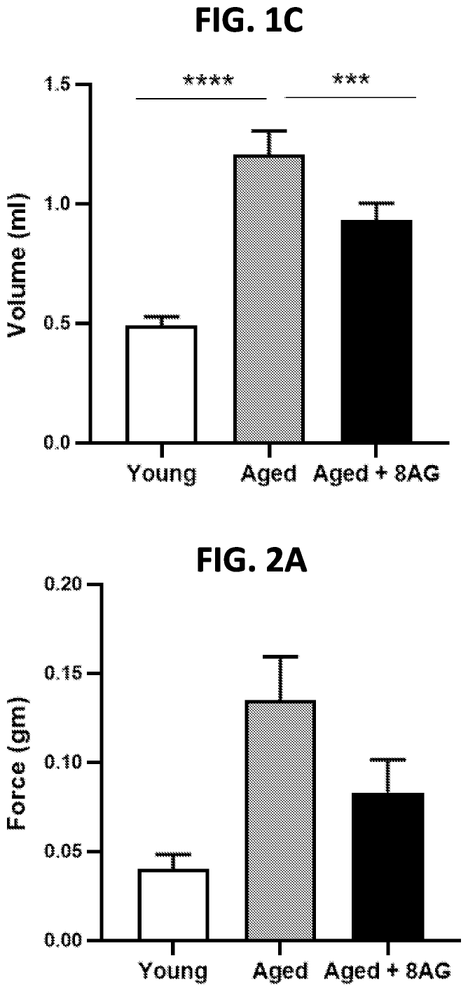

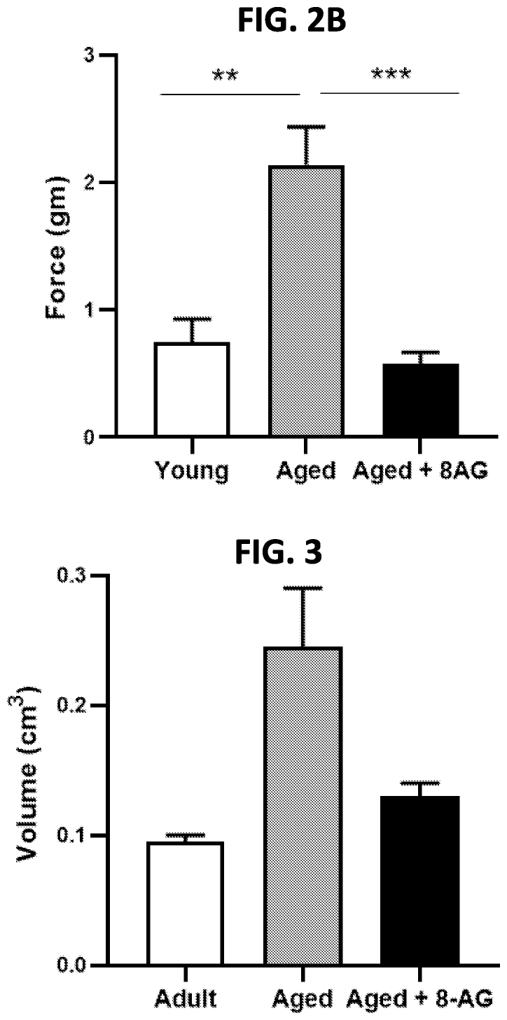

Results: As illustrated in FIGS. 1A-1C, aged rats (studied in metabolic cages) exhibited an increase in voided volume and a decrease in voiding efficiency and voiding frequency (FIGS. 1A-1C). These observations are consistent with preclinical and clinical studies that show increased bladder capacity and storage in aging adults. This was linked with a decrease in sensitivity to tactile (both abdominal and somatic) stimuli in aged rats (FIGS. 2A-2B). Both bladder and tactile abnormalities (FIGS. 1A-1C and 2A-2B) were reversed in aged rats by chronic treatment with 8-AG. Bladders from aged rats also exhibited a significant increase in bladder wall volume (FIG. 3), which was restored to that of a younger state by 8-AG treatment.

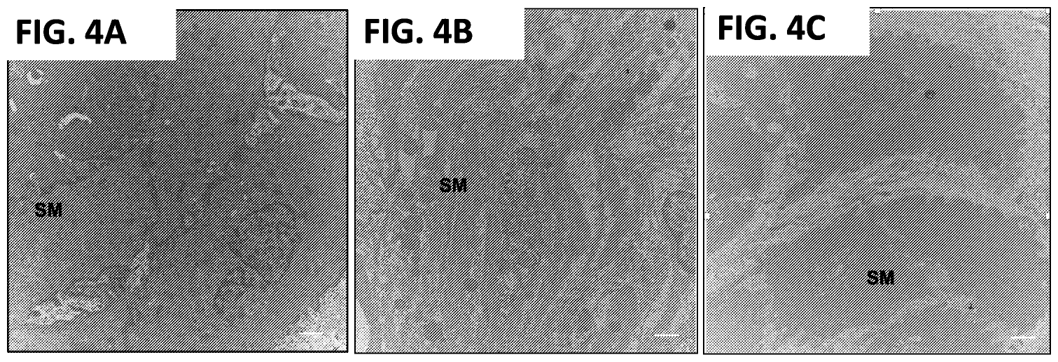

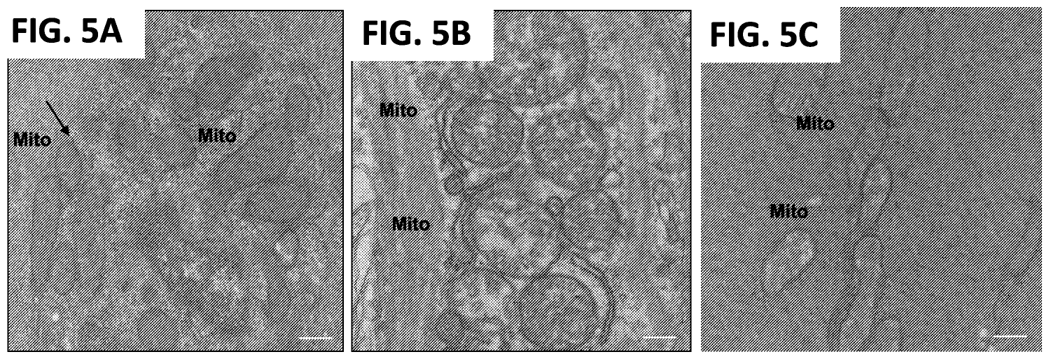

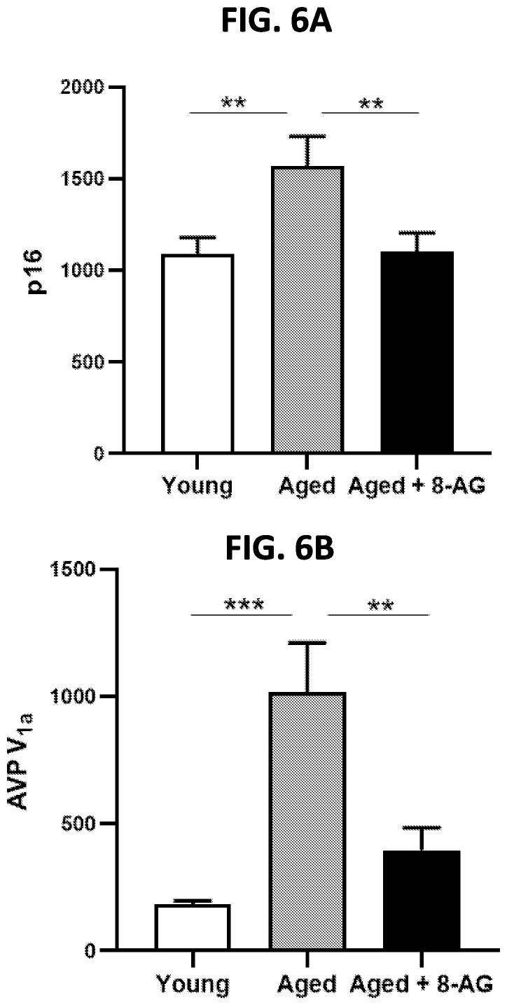

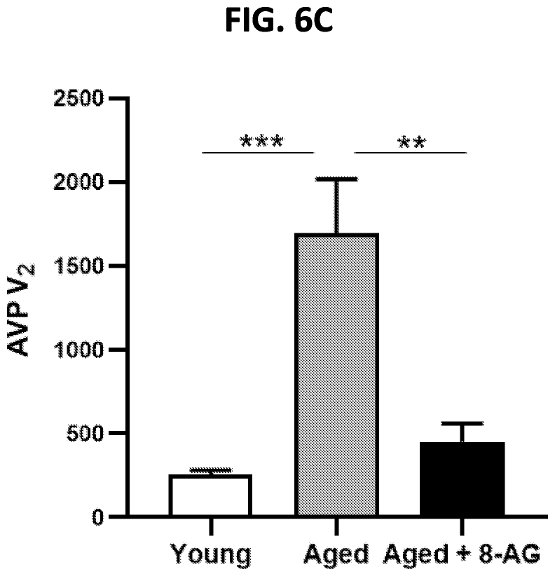

With 8-AG treatment in the aged rat, these increases were partially restored to a younger state. As illustrated in FIGS. 4A-4C, aging was associated with significant morphological changes in the urinary bladder smooth muscle showing a separation and degeneration of smooth muscle cells. Further, this structural pathology was accompanied by swelling and disruption of the smooth muscle mitochondria (FIGS. 5A-5C). In addition, increases in the cellular senescence marker p16 in the aged smooth muscle were observed (FIG. 6A). With 8-AG treatment, smooth muscle structural anomalies, including changes to the mitochondria and the senescence marker p16, were restored to levels similar to the younger state. A significant increase was observed in both AVP receptor V1a and V2 in the aged kidney (FIGS. 6B-6C). With 8-AG treatment, these receptor increases were restored to levels similar to the younger state.

### Example 2

Bladders that had been perfused transcardially with PBS and containing 20 nm yellow-green fluorescent beads (THERMO FISHER™, F8787) to highlight the vasculature were fixed then cleared rapidly by removing lipids and dissolving light-absorbing chromophores within the tissue. Clearing the tissue increased the maximum depth of confocal imaging to many millimeters. At this stage, tissue was directly imaged for the presence of genetically expressed reporter proteins or underwent staining using immunohistochemistry (IHC) or molecular dyes. Using this method, images were acquired through bladders (and other tissues) that are 5000 micrometers thick. Volumetric data are acquired using the Caliber ID RS-G4 ribbon scanning confocal microscope, which acquired large-area images 20-40 times faster than other commercially available systems with a high nA (1.0) long working distance (8 mm) low magnification (20×) by NIKON™ Inc (Tokyo, Japan). Quantitative changes in vascular structures were compared with the volume and surface area of each bladder and of the various tissue subtypes using these methods (UT; suburothelium; mesenchyme). These methods afforded the advantage of interrogating whole tissues in a three-dimensional environment and in combination with IHC without the need to section.

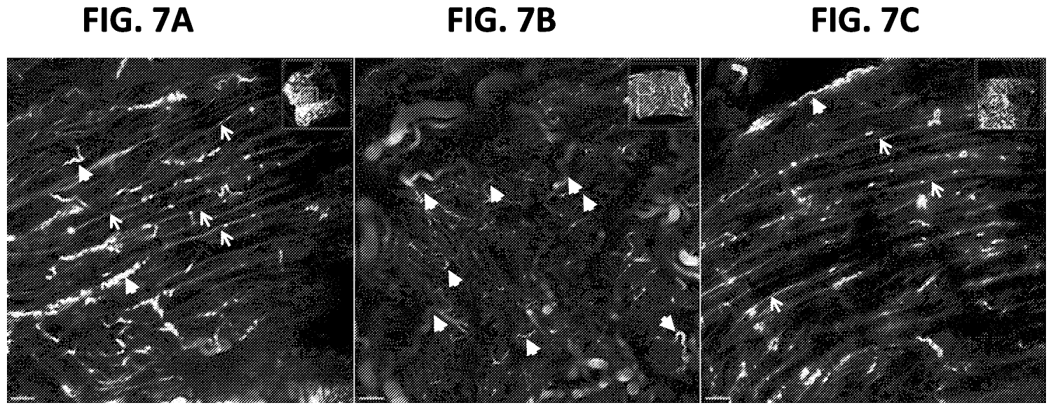

Results: Bladders perfused with fluorescent beads showed that young bladders were associated with mostly straight vessels throughout the tissue (FIG. 7A) in contrast to the aged bladder, which displays only tortuous vessels and in which the tissue appeared ischemic (FIG. 7B). Chronic treatment of aged rats with 8-AG (FIG. 7C) reduced the number of tortuous vessels, and the tissue no longer appeared ischemic, resembling young tissue.

### Example 3

Damage to the muscles or lack of hormonal stimulation are major contributing factors to urinary incontinence, which can lead to impaired bladder, emptying, urgency, and incontinence. The methods and materials used (such as for western immunoblotting and transmission electron microscopy) are the same as Example 1 with the tissues utilized for examination of muscle and mitochondrial morphology being the proximal urethral sphincter muscle and striated muscle of the external sphincter. Based on Examples 1 and 2, both muscle and mitochondrial morphology should show similar changes in the proximal urethral sphincter as well as the striated muscle of the external urethral sphincter and would contribute to lower urinary tract dysfunction.

### Example 4

Materials: 8-AG was purchased from Toronto Research Chemicals (Toronto, Ontario, Canada).

Animals: Male and female Fisher 344 rats were employed (Charles River; Wilmington, Mass. and the NIA rodent colony). Some rats were young (3 months of age), some were aged (25-30 months of age), and others were aged and treated with oral 8-AG (5 mg/kg/day; 6 weeks). Under isoflurane anesthesia and using an aseptic technique, an incision was made into the lower abdomen and in the scapular area. Sterile PE50 tubing was threaded subcutaneously from the lower abdomen to the scapular area and connected to a one-channel vascular access button (Intech Laboratories, Plymouth Meeting, Pa.). In the abdomen, the tubing was inserted into the bladder and secured with a small purse-string suture. The incisions were closed, and the animals were allowed to recover for 7-10 days before further experimentation. Animals were divided into three groups. A control group of aged rats received daily infusions with 10 μM DEA-NONOate (SIGMA-ALDRICH®, St. Louis, Mo.) in sterile 0.9% saline. A second group received 100 μM 8-AG plus 10 μM DEA-NONOate in 0.9% saline, and a third group received 5 μM forodesine (SIGMA-ALDRICH®) plus 10 μM DEA-NONOate in 0.9% saline. Bladders were instilled via a catheter tether kit (Instech Laboratories) connected to a syringe pump for continuous infusion (rate 200 μl/min; 30 minutes/daily) for 3-6 weeks. The Institutional Animal Care and Use Committee approved all procedures. The investigation conforms to the Guide for the Care and Use of Laboratory Animals published by the US National Institutes of Health (NIH Publication No. 85-23, revised 1996).

Protocol 1: Young, aged, and 8-AG-treated aged rats were placed in metabolism cages for 24 hr once per week. The light cycle was from 7:00 AM to 7:00 PM, and food and water were provided ad libitum. Voided urine was collected in cups attached to force displacement transducers (GRASS TECHNOLOGIES®, Warwick, R.I.) that were connected to a computer running Windaq data acquisition software (DATAQ® Instruments I2nc., Akron, Ohio). Data were averaged for 24 h and analyzed for 12-h periods during the day (7:00 AM-7:00 PM) and night (7:00 PM-7:00 AM). Voiding frequency, total voided volume, and volume per void were analyzed. Voiding frequency was calculated as the number of voiding events per hour during 24 h and during the 12-h day and 12-h night periods. Volume per void, which defines bladder capacity, was calculated as an average of the voids occurring during these periods.

Von Frey testing: Forty-eight h after the metabolism studies, tactile sensitivity was measured once per week using von Frey nylon filaments (Stoelting Co., Wood Dale, Ill., USA) applied to the suprapublic and plantar hindpaw. The median 50% withdrawal threshold (using an up-down method) with positive responses was recorded as sharp retraction, licking/scratching, or vocalization.

Western immunoblotting: Following the testing paradigms, rats were deeply anesthetized, bladders as well as the proximal urethra were removed, and one half of each preparation was homogenized using Lysing Matrix D in a FastPrep 24 instrument (MP Biomedicals, Solon, Ohio) in HBSS [5 mM KCl, 0.3 mM KH₂PO₄, 138 mM NaCl, 4 mM NaHO₃, 0.3 mM Na₂HCO₃, 0.3 mM Na₂HPO₄, 5.6 mM glucose, and 10 mM Hepes, pH 7.4 containing a complete protease inhibitor cocktail (1 tablet/10 ml, ROCHE®, Indianapolis, Ind.) and a phosphatase inhibitor cocktail (SIGMA®, 1:100)]. After centrifugation (13000 rpm; 15 min at 4° C.), the membrane protein fraction was prepared by suspending the membrane pellets in lysis buffer containing 0.3 M NaCl, 50 mM Tris-HCl (pH 7.6), and 0.5% Triton X-100 and the same concentration of protease inhibitors as above. The suspensions were incubated on ice and centrifuged (13000 rpm; 15 min at 4° C.). The protein concentrations of the combined supernatants were determined using the PIERCE® BCA protein assay (THERMO SCIENTIFIC®, Rockford, Ill.). After denaturation (100° C. for 5 min) in the presence of Laemmli sample buffer, lysate from each sample was separated on a 4-15% TGX Stain-Free SDS-PAGE gel (BIO-RAD® Laboratories, Hercules, Calif.). As a loading control, total protein per sample was determined using Bio-Rad Stain Free SDS-PAGE gel technology. UV-activated protein fluorescence was imaged on a CHEMIDOC® MP (BIO-RAD®). After the proteins were transferred to polyvinylidene fluoride membranes, the membranes were incubated in 5% (w/v) dried milk dissolved in TBS-T (20 mM Trizma, 137 mM NaCl, 0.1% Tween-20, pH 7.6), rinsed with TBS-T, and incubated overnight at 4° C. with primary antibody (α-SMA, cathepsin B, or LC3) diluted in TBS-T containing 5% (w/v) milk. After washing in TBS-T, the membranes were incubated with secondary antibody (Donkey anti-goat HRP; SOUTHERN BIOTECH®, Birmingham, Ala.) for 1 hour in 5% (w/v) Milk TBS-T, washed, and incubated in WesternBright Quantum (Advansta, Menlo Park, Calif.) and then imaged on a CHEMIDOC® MP (BIO-RAD®). The volume (intensity) of each protein species was determined and normalized to total protein using Image Lab software (BIO-RAD®).

Transmission electron microscopy (TEM): The other half of each urinary bladder, proximal urethra, or external urethral sphincter from each group (young, aged, aged+8-AG treatment) was fixed in cold 2.5% glutaraldehyde in 0.01 M PBS. The specimens were rinsed in PBS, post-fixed in 1% osmium tetroxide with 1% potassium ferricyanide, rinsed in PBS, dehydrated through a graded series of ethanol and propylene oxide solutions, and embedded in POLY/BED® 812 (Luft formulations). Semi-thin (300 nm) sections were cut on a LEICA® Reichart Ultracut, stained with 0.5% Toluidine Blue in 1% sodium borate and examined under the light microscope. Ultrathin sections (65 nm) were stained with uranyl acetate as well as Reynold's lead citrate and examined on a JEOL 1400 transmission electron microscope with a side mount AMT 2k digital camera (Advanced Microscopy Techniques, Danvers, Mass.).

Protocol 2: Purines in urine were measured by mass spectrometry using selected reaction monitoring. Urine samples were diluted 1 to 30 with water, and heavy isotope internal standards were added to each sample. Purines were separated by reversed-phase ultra-performance liquid chromatography (Waters UPLC BEH C18 column, 1.7 μm beads; 2.1×150 mm; Milford, Mass.) and quantified by selected reaction monitoring using a triple quadrupole mass spectrometer (TSQ Quantum-Ultra; THERMOFISHER SCIENTIFIC®, San Jose, Calif.) with a heated electrospray ionization source. The mobile phase was a linear gradient flow rate (300 μL/min) of 1% acetic acid in water (pH, 3; mobile phase A) and 100% methanol (mobile phase B) and was delivered with a Waters Acquity ultra-performance liquid chromatographic system. The gradient (A/B) settings were as follows: from 0 to 2 minutes, 99.6%/0.4%; from 2 to 3 minutes, to 98.0%/2.0%; from 3 to 4 minutes, to 85.0%/15.0%; and from 4 to 6.5 minutes, to 99.6%/0.4%. The instrument parameters were: sample tray temperature, 10° C.; column temperature, 50° C.; ion spray voltage, 4.0 kilovolts; ion transfer tube temperature, 350° C.; source vaporization temperature, 320° C.; Q2 CID gas, argon at 1.5 mTorr; sheath gas, nitrogen at 60 psi; auxillary gas, nitrogen at 35 psi; Q1/Q3 width, 0.7/0.7 units full-width half-maximum; scan width, 0.6 units; scan time, 0.01 seconds. The following transitions (selected reaction monitoring) were obtained: hypoxanthine (137→119 m/z, RT=1.86 min); ¹³C₅-hypoxanthine (142→124 m/z, RT=1.86 min); 8-AG (167→150 m/z, RT=1.50 min); ¹³C₂¹⁵N-aminoguanine (170→153 m/z, RT=1.50 min).

Protocol 3: Planar biaxial mechanical testing coupled with multiphoton microscopy was performed on bladder wall specimens to assess bladder mechanical function while simultaneously observing changes in the collagen microstructure. Briefly, the unloaded intact bladder diameter (dₒ) and meridional length (hᵥ) were measured and used to calculate the unloaded bladder volume (V₀) from the ellipsoid volume, V₀=πd₀²h₀/6. The intact bladders were then cut open longitudinally and trimmed into 6±1 mm×6±1 mm square specimens for mechanical testing with sides aligned in the in situ longitudinal and circumferential directions. Samples were then positioned in the custom-designed biaxial testing system and mechanically tested using fiducial markers on the sample to calculate stretch, defined as the ratio of distance between markers in sample when loaded and unloaded. A multiphoton microscope (OLYMPUS® FV1000 MPE) equipped with a Coherent Cheleon TiSapphire pulsed Laser was used to image the undulated (wavy) collagen fibers in the mounted samples without staining or fixation during loading (i.e., stretch) as per previously published methods. Stacks of 2D planar images were generated by imaging sequentially across the wall thickness. To avoid tissue damage while obtaining a large range of stretch, loading was stopped at the stretch where collagen fibers were visibly straightened (heretofore termed recruited). This was defined as the maximum stretch. Fiber tortuosity was measured by tracing collagen fibers across the 2D slices (Filament function in Imaris, Bitplane, Switzerland). Fiber arc length (s) and cord length (L) were determined for each fiber tracing and used to calculate the tortuosity. The tortuosity of an undulated or wavy fiber is, therefore, greater than one and approaches one as the fiber becomes fully straightened.

Protocol 4: Intravesical instillation of either Krebs buffer alone or hypoxanthine (30 μM in Krebs buffer solution; 400 μl; 3 hours, SIGMA-ALDRICH®) was performed in urethane anesthetized animals via a urethral catheter (with ureters tied). The solution was removed using a 25 G needle connected to a syringe inserted via the bladder dome. Measurement of the biomarker for oxidative stress, 8-isoprostane, was performed using ELISA method (8-iso-PGFa, Enzo Life Sciences, Farmingdale N.Y.). Similar 8-isoprostane measurements were performed on urines from patients with urinary symptoms compared to healthy controls.

Statistics: Data were analyzed in GRAPHPAD® PRISM® 6 (GRAPHPAD®, La Jolla, Calif.) using Student's t-test and one-way ANOVA followed by appropriate post-hoc tests. P<0.05 was considered significant. The results are expressed as means±SEM.

### Example 5

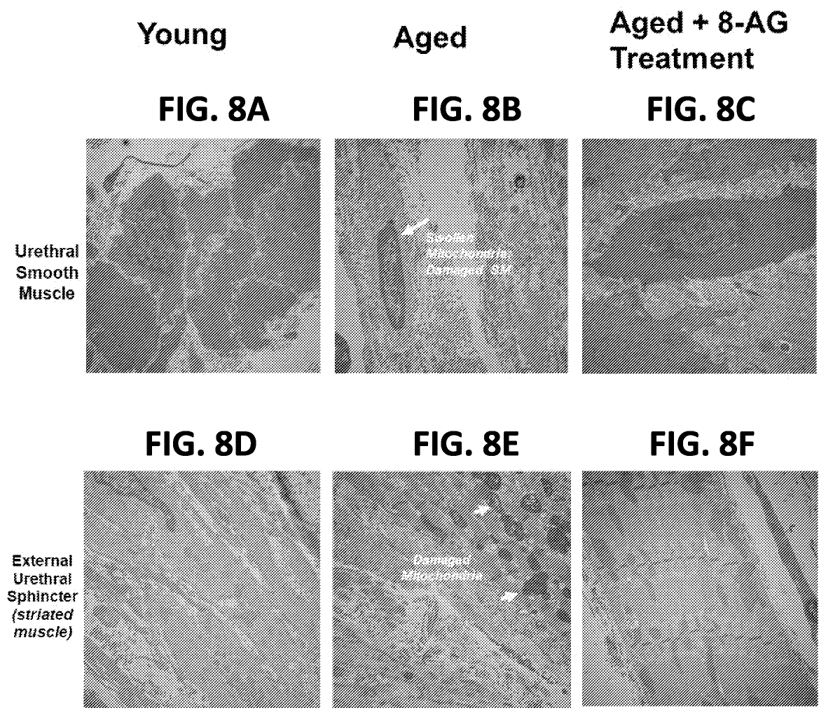

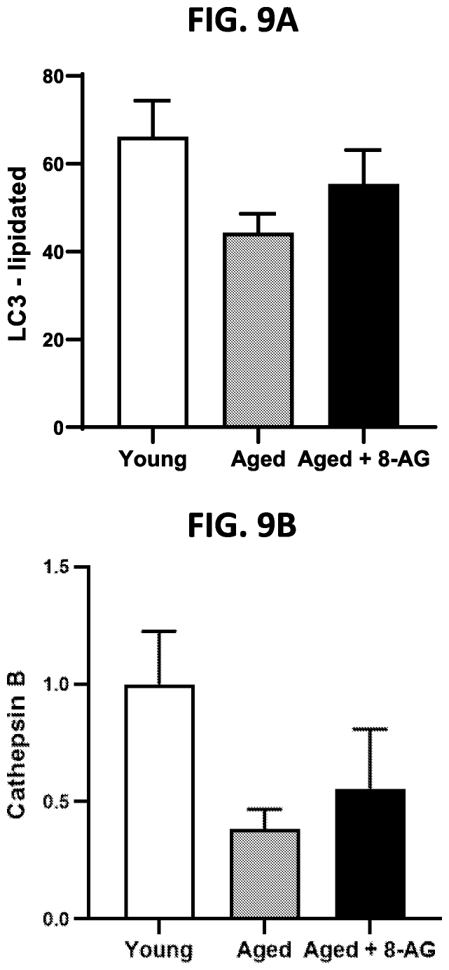

As illustrated in FIGS. 8A-8F, aging was associated with significant morphological changes in the urethral smooth muscle (compare FIG. 1A with FIG. 1B) as well as the striated muscle (compare FIG. 8D with FIG. 8E; urethral external sphincter). This structural pathology was accompanied by swelling and disruption of the smooth and striated muscle mitochondria. With oral 8-AG treatment, the smooth and striated muscle abnormalities, including changes to the mitochondria, were reversed (FIGS. 8C and 8F). In support are findings (FIGS. 9A-9B) that show a decrease in the lipidated form of LC3 (correlated with induction of autophagy, FIG. 9A) in aged detrusor and a decrease in the enzyme cathepsin B (involved in lysosomal function, FIG. 9B) in aged urethra. With oral 8-AG treatment, there was a trend toward reversal in both proteins that of a younger state.

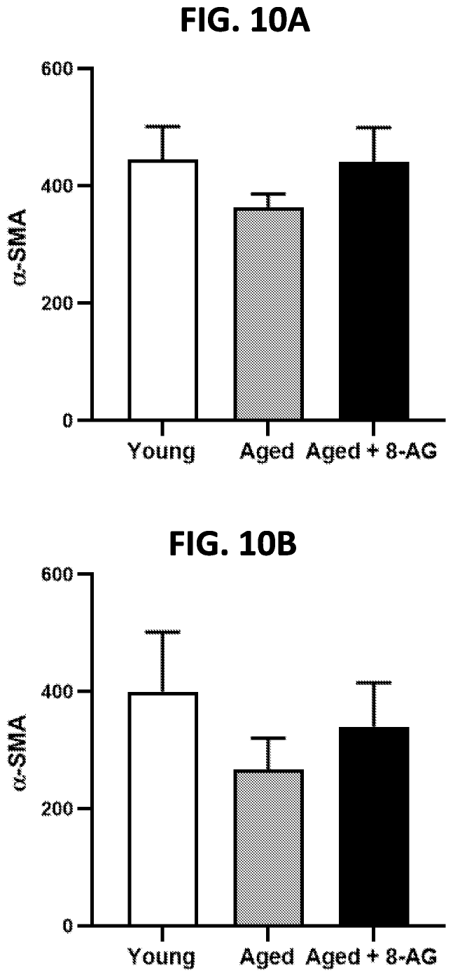

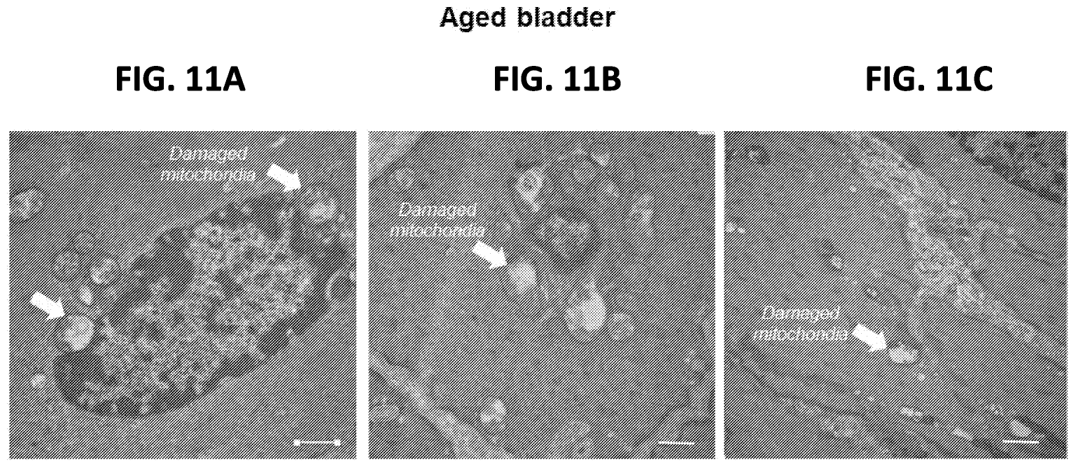

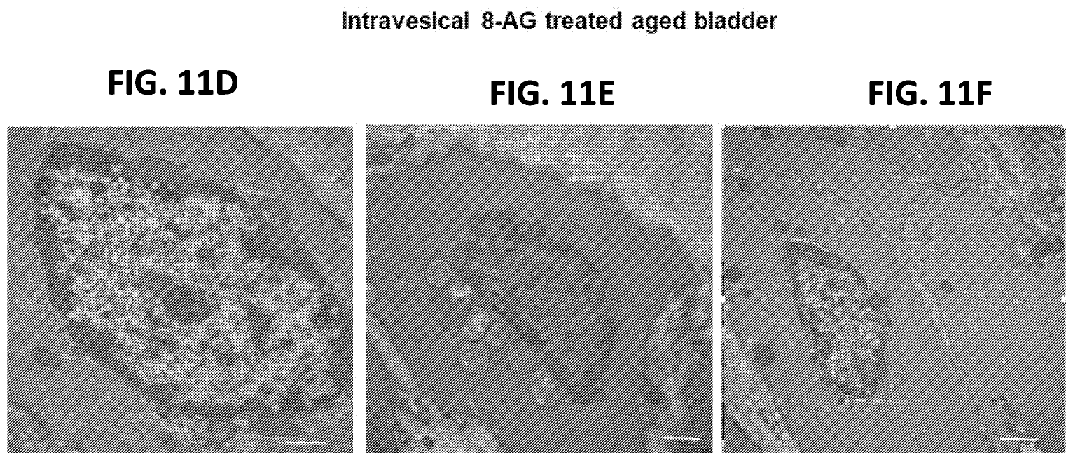

In addition, both bladder and urethral smooth muscle expression changed (as measured by western blotting for α-SMA) in aged bladders, which was normalized with 8-AG oral treatment (FIGS. 10A-10B). Further, intravesical 8-AG treatment also restored urinary bladder smooth muscle mitochondrial abnormalities (FIGS. 11A-11F).

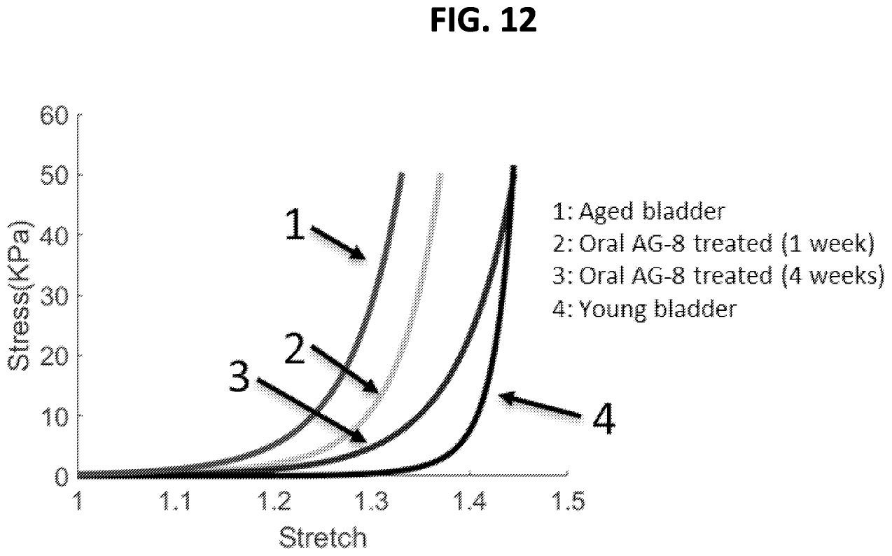

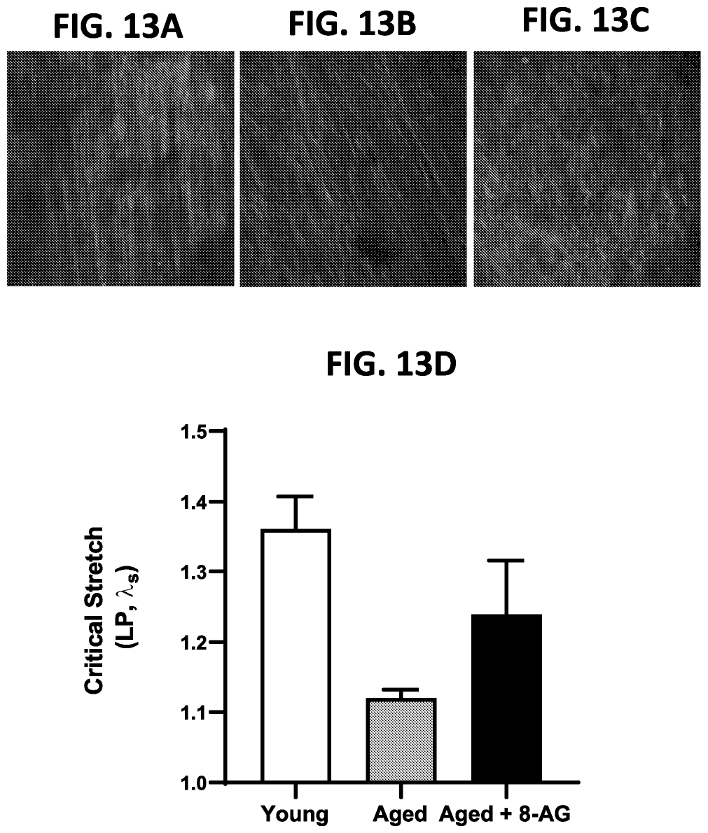

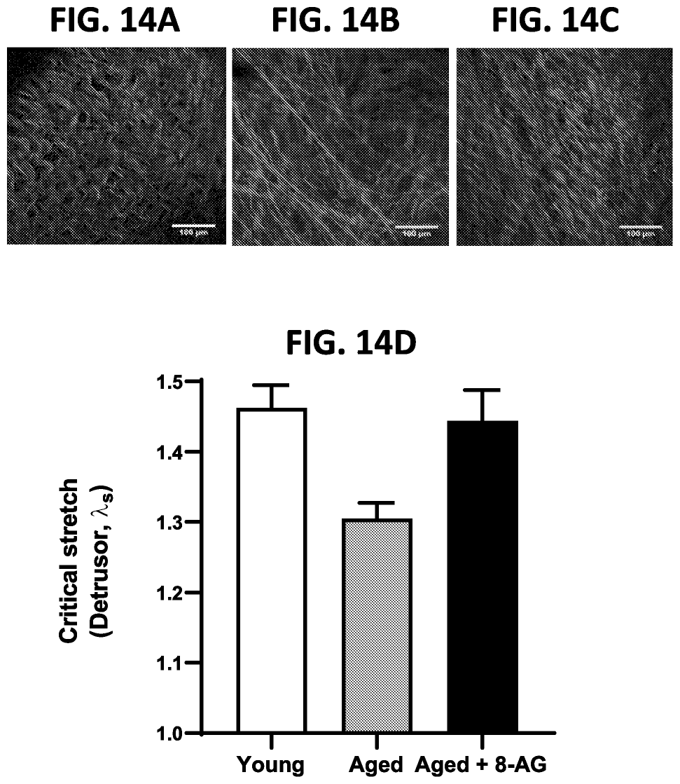

As illustrated in FIG. 12, in all bladders the mechanical response to stretch, as shown in the stress-stretch curve, displays an initial ‘soft’ or compliant phase necessary for bladder filling in which the bladder wall can extend with little change in stress. Once a threshold of stretch is reached (FIG. 12), a ‘stiff’ or noncompliant phase is observed in which the bladder rapidly stiffens, thus, setting the maximum stretch achievable during physiological loading. The stretch at which this steep increase in stiffness occurs depends on the tortuosity of the collagen fibers. In this regard, the tortuosity enables bladder filling at low mechanical loads (or pressure) because fibers contribute little to load bearing until they are straightened (‘recruited’). As increasing numbers of fibers are recruited, the bladder rapidly stiffens. With age, collagen fiber tortuosity is diminished, which means that a substantial fraction of fibers is highly straightened in both the lamina propria (compare FIG. 13A with FIG. 13B) and detrusor layers (compare FIG. 14A with FIG. 14B). This diminished tortuosity with age results in a leftward shift of the bladder's stress-stretch relationship (FIG. 12) that manifests as a substantial reduction in the stretch at which the stiff phase for bladder occurs in aged rats relative to young rats (FIGS. 13D and 13D). After treatment with 8-AG, fiber tortuosity in both layers is increased (FIGS. 13C and 14C), corresponding to a larger fraction of highly tortuous fibers that allow increased filling at a lower pressure. This results in a more extensible (or compliant) bladder (FIG. 12). 8-AG treatment impacts both the first and second transition points, indicating that the treatment restores collagen fiber tortuosity in the lamina propria (FIG. 13D) as well as the detrusor layers (FIG. 14D), which results in a more compliant bladder closer to a younger state.

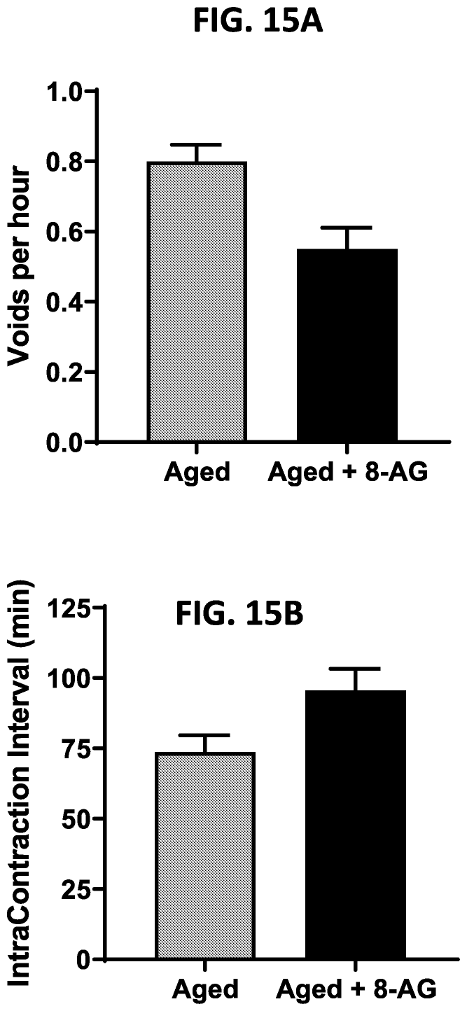

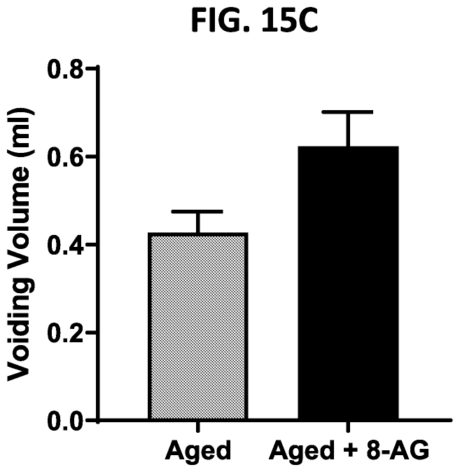

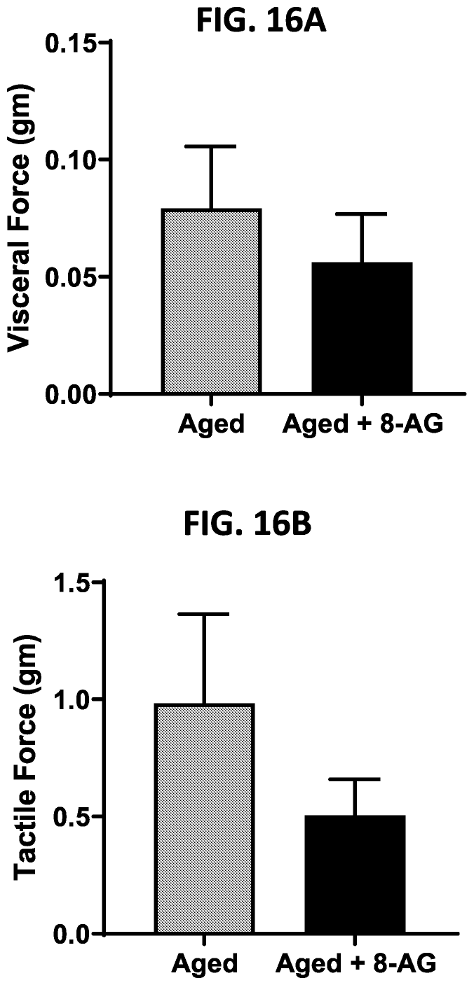

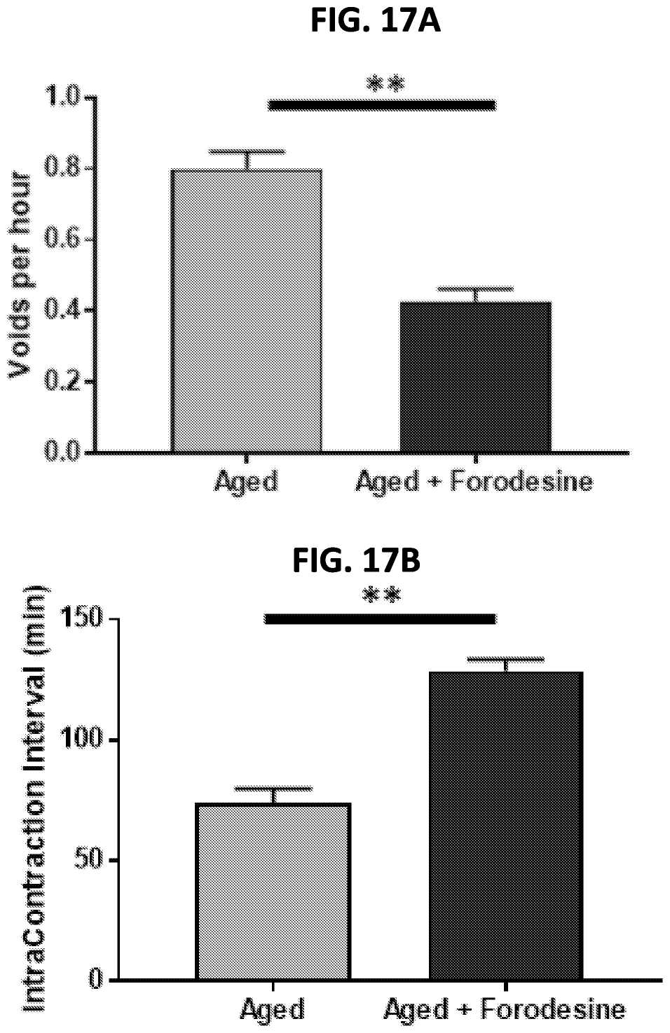

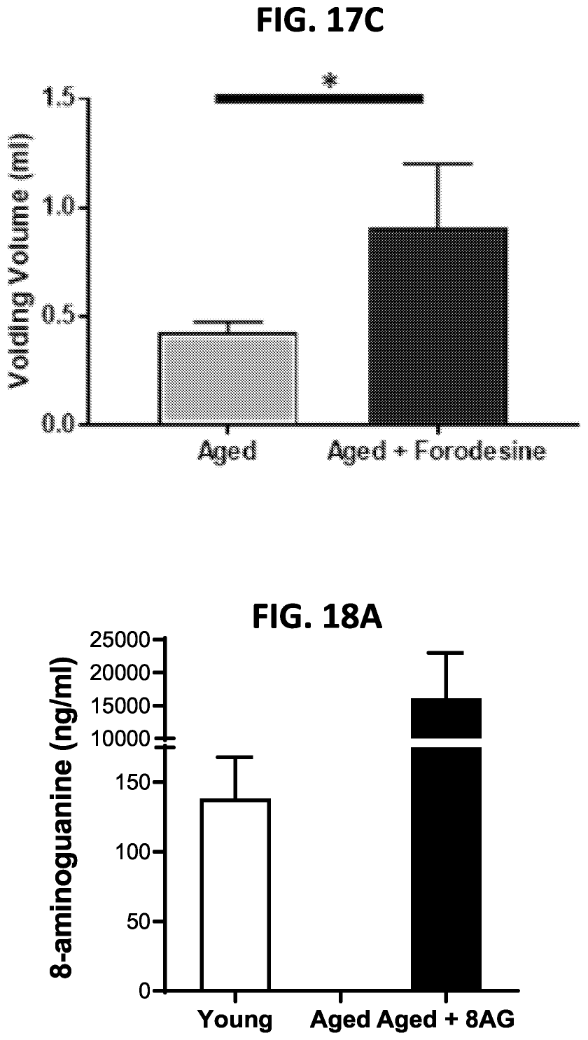

As illustrated in FIGS. 15A-15C, intravesical treatment of aged rats with 8-AG decreased voiding frequency (FIG. 15A) increased the inter-contraction voiding interval (FIG. 15B) and increased voided volume (FIG. 15C) compared with control aged rats. With intravesical 8-AG treatment in aged rats, there was also an increase in sensitivity to tactile (both abdominal and somatic) stimuli (FIGS. 16A-16B). A significant improvement in age-associated bladder voiding function was also observed (FIGS. 17A-17C) with intravesical instillation of a highly potent PNPase inhibitor forodesine (voiding frequency, inter-contraction interval, and voided volume) compared with aged control rats.

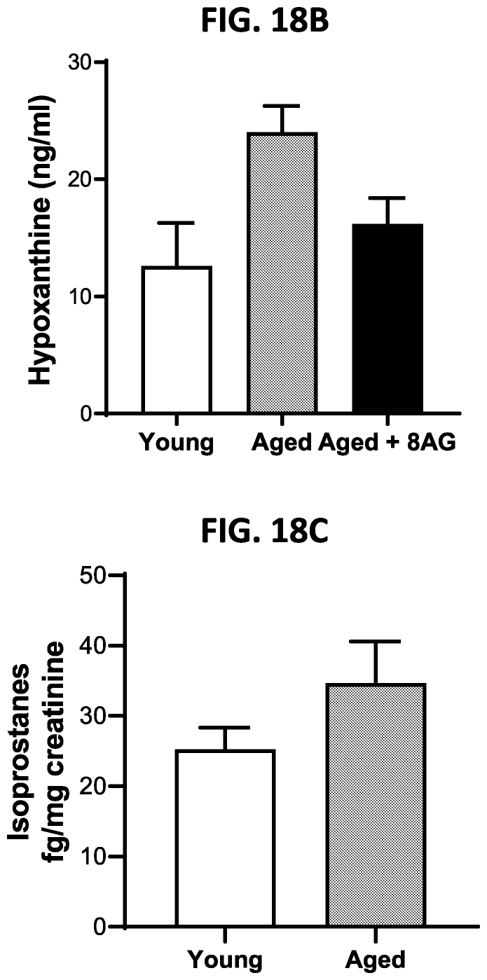

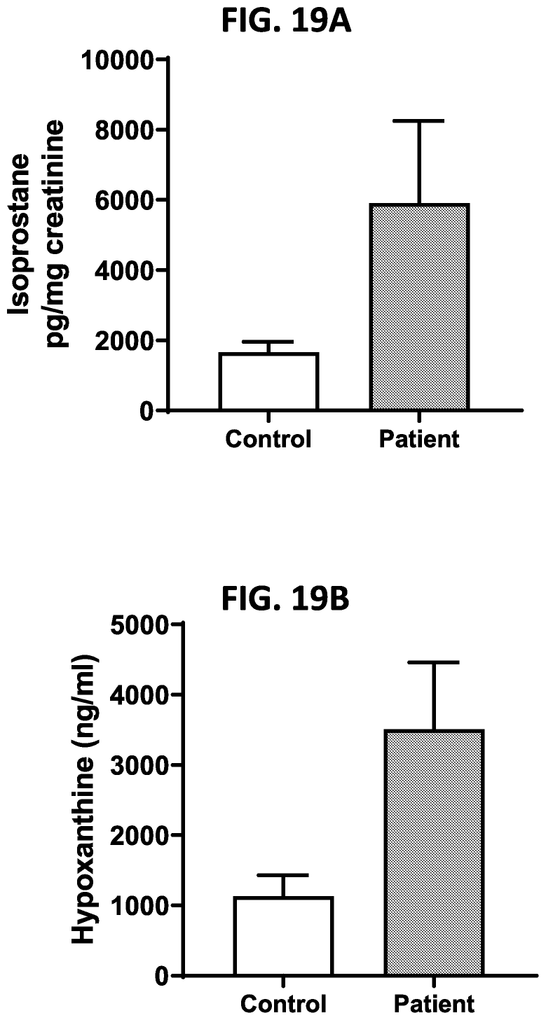

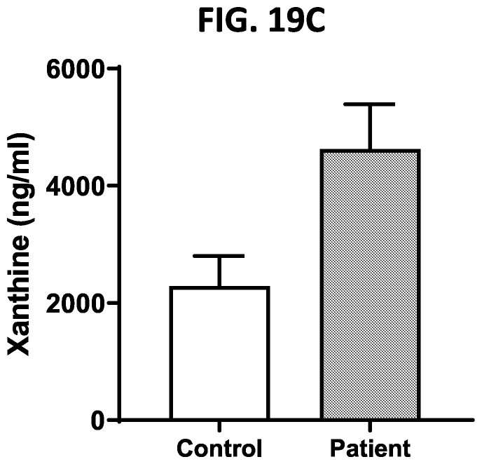

As shown in FIG. 18A, aged rats excrete undetectable amounts of endogenous 8-AG, which is restored to younger levels following oral 8-AG treatment. Further, urinary hypoxanthine levels were higher in aged rats compared with young rats, and oral 8-AG significantly reduced urinary hypoxanthine to levels comparable to younger rats (FIG. 18B). Further, intravesical instillation of hypoxanthine (30 μM) increased a biomarker for oxidative stress (8-isoprostane) in both young and aged bladders; however, the increase was substantially greater in aged rats (FIG. 18C). Moreover, aged patients exhibit an increase in urinary 8-isoprostanes (FIG. 19A), hypoxanthine (FIG. 19B), xanthine (FIG. 19C), and urinary symptoms compared with young, healthy controls (FIGS. 19A-19C).

### Example 5—Methods and Materials (for Example 6)

Materials: The 8-AG was purchased from Toronto Research Chemicals (Toronto, Ontario, Canada).

Animals: This example employed male and female young (3 mo) and aged (25-30 mo) Fischer 344 rats (Charles River; Wilmington, Mass. and the NIA rodent colony). Some aged rats were treated with oral 8-AG (5 mg/kg/day; 6 weeks). Human Subjects: De-identified urine samples were obtained from patients (recruited from patients receiving their care from urologic clinics) with voiding symptoms and age- and sex-matched healthy controls.

Voiding Analysis: Young, aged, and 8-AG-treated aged rats were placed in metabolic cages (24 hr; once per week) for the duration of the study. The light cycle was from 7:00 AM to 7:00 PM, and food and water were provided ad libitum. Voided urine was collected in cups attached to force displacement transducers (Grass Technologies, Warwick, R.I.) connected to a computer (Windaq data acquisition software DATAQ Instruments Inc., Akron, Ohio). Data were averaged for 24 h and analyzed for 12-h periods during the day (7:00 AM-7:00 PM) and night (7:00 PM-7:00 AM). Voiding frequency, total voided volume, and volume per void were analyzed. Voiding frequency was calculated as the number of voiding events per hour during 24 hr and during the 12-hr day and 12-hr night periods. Volume per void, which defines bladder capacity, was calculated as an average of the voids occurring during these periods.

Von Frey Testing: Forty-eight hr after the metabolic cage studies, tactile sensitivity was measured once per week for the duration of the study using von Frey nylon filaments (Stoelting Co., Wood Dale, Ill., USA) applied to the suprapublic and plantar hindpaw. The withdrawal threshold (using up-down method) was determined, with positive responses defined as sharp retractions, licking/scratching, or vocalizations.

Vascular Changes: Bladders that had been perfused transcardially with PBS buffer containing 20 nm yellow-green fluorescent beads (THERMO FISHER®, F8787) to highlight the vasculature were fixed then cleared by removing lipids and dissolving light-absorbing chromophores within the tissue. Volumetric data were acquired using a Caliber ID RS-G4 ribbon scanning confocal microscope (NIKON® Inc, Tokyo, Japan), which can acquire large-area images (uses a high nA (1.0) long working distance (8 mm) with low magnification (20×)). This affords the advantage of interrogating whole tissues in a three-dimensional environment without the need to section. Real-time blood perfusion (1 mm3 tissue) was accomplished using a BLF22D laser Doppler flowmeter (Transonic Systems, Inc) with a surface probe (TypeS-APLPHS) applied to the serosal surface of the bladder (apex and neck) wall using Doppler light shift from moving RBCs to analyze flow by the Bonner algorithm. This method gives robust, non-invasive microvascular flow signals in the bladder wall of anesthetized rats.

Bi-axial Stretch Combined with Multi-Photon (MPM) Imaging: Planar biaxial mechanical testing coupled with multiphoton microscopy was performed on bladder wall specimens to assess bladder mechanical function while simultaneously observing changes in the collagen microstructure. Briefly, the unloaded intact bladder diameter (dₒ) and meridional length (hᵥ) were measured and used to calculate the unloaded bladder volume (V₀) from the ellipsoid volume, V₀=πd₀²h₀/6. The intact bladders were then cut open longitudinally and trimmed into 6±1 mm×6±1 mm square specimens for mechanical testing with sides aligned in the situ longitudinal and circumferential directions. Samples were then positioned in a biaxial testing system and mechanically tested using fiducial markers on the sample to calculate stretch, defined as the ratio of distance between markers in sample when loaded and unloaded. A multiphoton microscope (OLYMPUS® FV1000 MPE) equipped with a Coherent Cheleon TiSapphire pulsed Laser was used to image the undulated (tortuous or wavy) collagen fibers in the mounted samples without staining or fixation during loading (i.e., stretch). Stacks of 2D planar images were generated by imaging sequentially across the wall thickness. To avoid tissue damage while obtaining a large range of stretch, loading was stopped at the stretch where collagen fibers were visibly straightened (heretofore termed recruited). This was defined as the maximum stretch. Fiber tortuosity was measured by tracing collagen fibers across the 2D slices (Filament function in Imaris, Bitplane, Switzerland). Fiber arc length (s) and cord length (L) were determined for each fiber tracing and used to calculate the tortuosity. The tortuosity of an undulated or wavy fiber is, therefore, greater than one and approaches one as the fiber becomes fully straightened.

Transmission electron microscopy (TEM): Half of each urinary bladder from each group (young, aged, aged+8-AG treatment) was fixed in cold 2.5% glutaraldehyde in 0.01 M PBS. The specimens were rinsed in PBS, post-fixed in 1% osmium tetroxide with 1% potassium ferricyanide, rinsed in PBS, dehydrated through a graded series of ethanol and propylene oxide solutions, and embedded in POLY/BED® 812 (Luft formulations). Semi-thin (300 nm) sections were cut on a LEICA® Reichart Ultracut, stained with 0.5% Toluidine Blue in 1% sodium borate, and examined under the light microscope. Ultrathin sections (65 nm) were stained with uranyl acetate and Reynold's lead citrate and examined on a JEOL 1400 transmission electron microscope with a side mount AMT 2k digital camera (Advanced Microscopy Techniques, Danvers, Mass.).

Western immunoblotting: One half of each bladder preparation was homogenized using Lysing Matrix D in a FastPrep 24 instrument (MP Biomedicals, Solon, Ohio) in HBSS (5 mM KCl, 0.3 mM KH₂PO₄, 138 mM NaCl, 4 mM NaHO₃, 0.3 mM Na₂HCO₃, 0.3 mM Na₂HPO₄, 5.6 mM glucose and 10 mM Hepes, pH 7.4) containing complete protease inhibitor cocktail (1 tablet/10 ml, ROCHE®, Indianapolis, Ind.)) and phosphatase inhibitor cocktail (SIGMA®, 1:100). After centrifugation (13000 rpm; 15 min at 4° C.), the membrane protein fraction was prepared by suspending the membrane pellets in lysis buffer containing 0.3 M NaCl, 50 mM Tris-HCl (pH 7.6) and 0.5% Triton X-100 and the same concentration of protease inhibitors as above. The suspensions were incubated on ice and centrifuged (13000 rpm; 15 min at 4° C.). The protein concentrations of the combined supernatants were determined using the PIERCE® BCA protein assay (THERMO SCIENTIFIC®, Rockford, Ill.). After denaturation (100° C. for 5 min) in the presence of Laemmli sample buffer, lysate from each sample was separated on a 4-15% TGX Stain-Free SDS-PAGE gel (BIO-RAD® Laboratories, Hercules, Calif.). As a loading control, total protein per sample was determined using Bio-Rad® Stain Free SDS-PAGE gel technology. UV-activated protein fluorescence was imaged on a CHEMIDOC® MP (BIO-RAD®). After proteins were transferred to polyvinylidene fluoride membranes, the membranes were incubated in 5% (w/v) dried milk dissolved in TBS-T (20 mM Trizma, 137 mM NaCl, 0.1% Tween-20, pH 7.6), rinsed with TBS-T, and incubated overnight at 4° C. with primary antibody (e.g., α-SMA (NOVEX® 701457), SMMHC (smooth muscle myosin heavy chain, ProteinTech 21404-1-AP), senescent marker p16 (ABCAM® AB51243), mitofusin 2 (MFN2, ABCAM® AB56889), DRP-1 (dynamin-related protein 1, Cell Signaling 8570), Parkin (Cell Signaling 4211), caspase 3 (Cell Signaling, cleaved 9664, total 9665), catalase (ABCAM® AB83464 and AB76110), PARP (Cell Signaling 9542), nitrotyrosine (Cell Signaling 9691), and cathepsin B (Cell Signaling 31718)) diluted in TBS-T containing 5% (w/v) milk. After washing in TBS-T, the membranes were incubated with secondary antibody (Sheep anti-mouse HRP; SOUTHERN BIOTECH®, Birmingham, Ala. or Donkey anti-rabbit HRP; Advansta, San Jose, Calif.) for 1 hour in 5% (w/v) Milk TBS-T, washed, incubated in WESTERNBRIGHT® Quantum (Advansta, Menlo Park, Calif.), and then imaged on a CHEMIDOC® MP (BIORAD®). The volume (intensity) of each protein species was determined and normalized to total protein using Image Lab software (BIO-RAD®).

Purine Metabolome Measurement: Purines in urine were measured by mass spectrometry using selected reaction monitoring.(7,13) Urine samples were diluted 1 to 30 with water, and heavy isotope internal standards were added to each sample. Purines were separated by reversed-phase ultra-performance liquid chromatography (Waters UPLC BEH C18 column, 1.7 μm beads; 2.1×150 mm; Milford, Mass.) and quantified by selected reaction monitoring using a triple quadrupole mass spectrometer (TSQ Quantum-Ultra; THERMOFISHER SCIENTIFIC®, San Jose, Calif.) with a heated electrospray ionization source. The mobile phase was a linear gradient flow rate (300 uL/min) of 1% acetic acid in water (pH, 3; mobile phase A) and 100% methanol (mobile phase B), and was delivered with a Waters Acquity ultra-performance liquid chromatographic system. The gradient (A/B) settings were: from 0 to 2 minutes, 99.6%/0.4%; from 2 to 3 minutes, to 98.0%/2.0%; from 3 to 4 minutes, to 85.0%/15.0%; and from 4 to 6.5 minutes, to 99.6%/0.4%. The instrument parameters were as follows: sample tray temperature, 10° C.; column temperature, 50° C.; ion spray voltage, 4.0 kilovolts; ion transfer tube temperature, 350° C.; source vaporization temperature, 320° C.; Q2 CID gas, argon at 1.5 mTorr; sheath gas, nitrogen at 60 psi; auxillary gas, nitrogen at 35 psi; Q1/Q3 width, 0.7/0.7 units full-width half-maximum; and scan width, 0.6 units; scan time, 0.01 seconds. The following transitions (selected reaction monitoring) were obtained: hypoxanthine (137→119 m/z, RT=1.86 min); ¹³C₅-hypoxanthine (142→124 m/z, RT=1.86 min); 8-AG (167→150 m/z, RT=1.50 min); ¹³C₂¹⁵N-8-AG (170→153 m/z, RT=1.50 min).

Statistics: Data were analyzed in GRAPHPAD® PRISM® 6 (GRAPHPAD®, La Jolla, Calif.) using Student's t-test and one-way ANOVA followed by appropriate post-hoc tests. P<0.05 was considered significant. The results are expressed as means±SEM.

### Example 6—Results

The effects of aging on the lower tract are complex (Pfisterer et al., JAGS, 54:405-412, 2006; Dubeau, J Urol, 175:S11-15, 2006). Studies in animals show that multiple components in the bladder become dysfunctional with age (Klullmann et al., Clin Geriatr Med, 31:535-548, 2015). In this regard, aging impacts bladder mucosal, muscular, and neural components in differing manners and to differing extents. Common pathological features likely arise from: 1) vascular changes (ischemia associated with reperfusion injury, which is similar to that seen in the myocardium); 2) mucosal pathology (increases in mucosal permeability and loss of mucosal cells with effects on the local cell-cell and cell-interstitium communications necessary for normal sensation and regulation of bladder wall inflammation); and 3) bladder muscular storage and contractile dysfunction (resulting from vascular, neural and other pathologic factors). These pathological changes converge to cause voiding symptoms including urgency, urinary incontinence, impaired bladder contractility, nocturia and decreased sensation (often resulting in incomplete emptying; Gibson and Wagg, Nat Rev Urol, 14:440-448, 2017). These symptoms are often grouped into overlapping sets, with each set designated as a particular named “LUT disorder” (LUTD). The demographics of LUTDs suggest a rapid increase in their occurrence of these symptoms and their underlying causative etiologies beginning in the fifth decade in both genders, increasing until the end of life and effecting at least 30% in aggregate of the post-50-year old population (Aldamahori and Chappel, Curr Opin Urol: 27:293-299, 2017; Wein and Moy, Campbell-Walsh Urology, W.B. Saunders, Philadelphia, 2011-2044, 2007).

The bladders of aged rats exhibited a number of abnormalities: bladder and tactile, vascular remodeling, collagen fiber tortuosity (increases bladder stiffness), morphological smooth muscle changes (bladder and urethra), swelling of mitochondria and increases in uro-damaging purine metabolites. Treatment of aged rats with 8-AG normalized all abnormalities to that of a younger state. It was demonstrated that 8-AG, a potent inhibitor of PNPase, reverses age-related morphological, biochemical and functional changes in the older bladder. The examples herein findings support 8-AG reversal of age-induced LUT disorders.

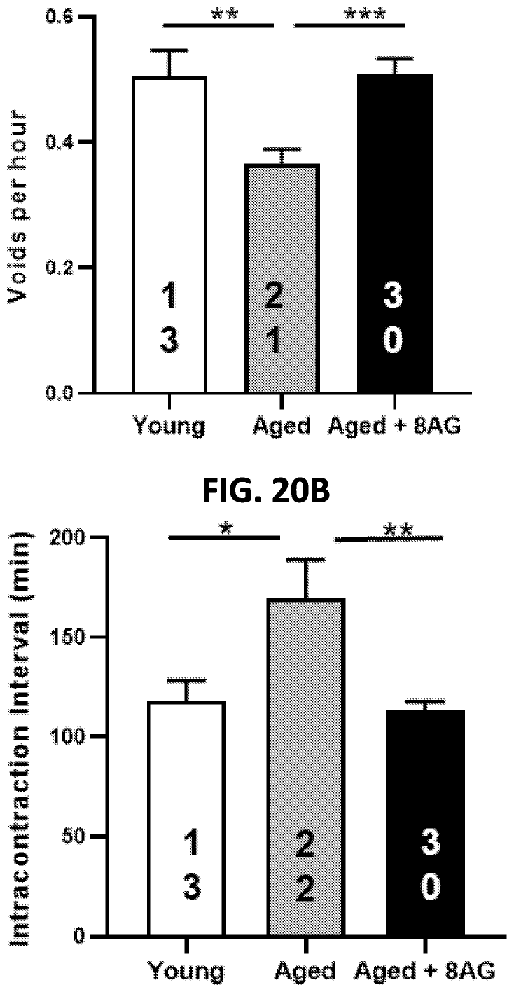

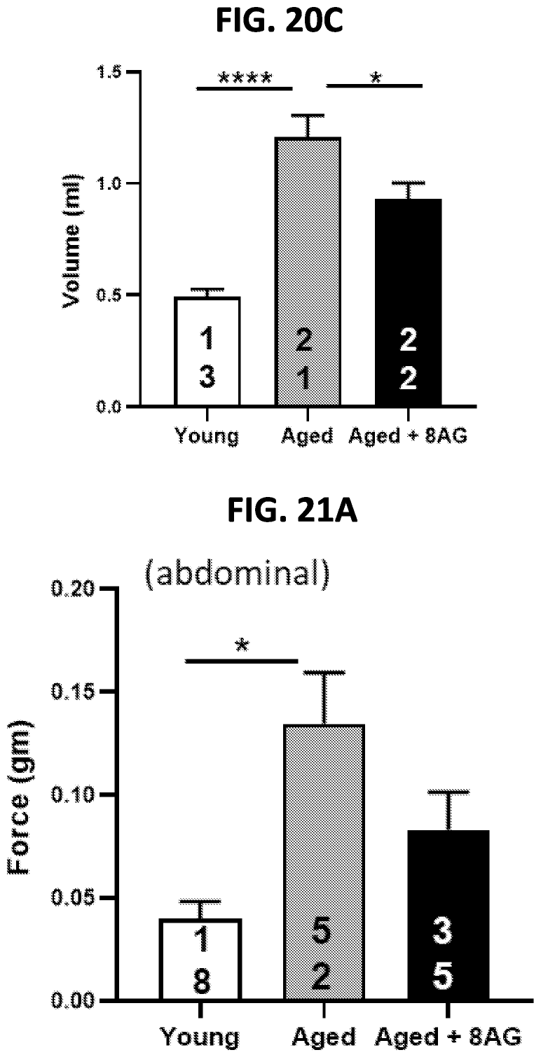

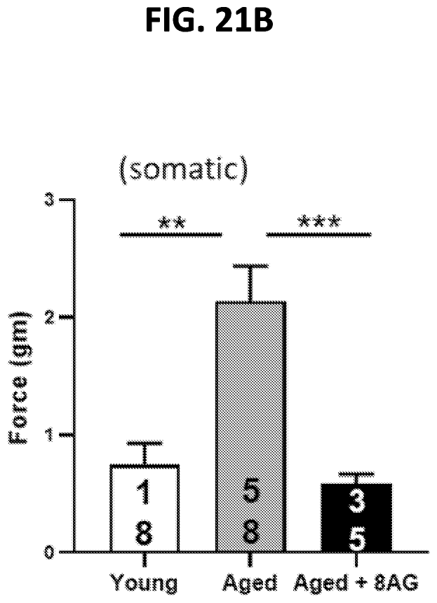

Untreated aged rats (studied in metabolic cages) exhibited a decrease in voiding frequency (FIG. 20A) and increases in the intercontraction interval (FIG. 20B) as well as voided volume (FIG. 20C). These observations are consistent with preclinical and clinical studies that show increased bladder capacity and storage in aging adults and are characteristic of an underactive bladder. This observation was linked with a decrease in von Frey sensitivity to tactile stimuli, both abdominal (FIG. 21A) and somatic (FIG. 21B), in aged rats. However, in aged rats treated chronically with 8-AG, both voiding behavior and tactile sensitivity were similar to those observed in young rats.

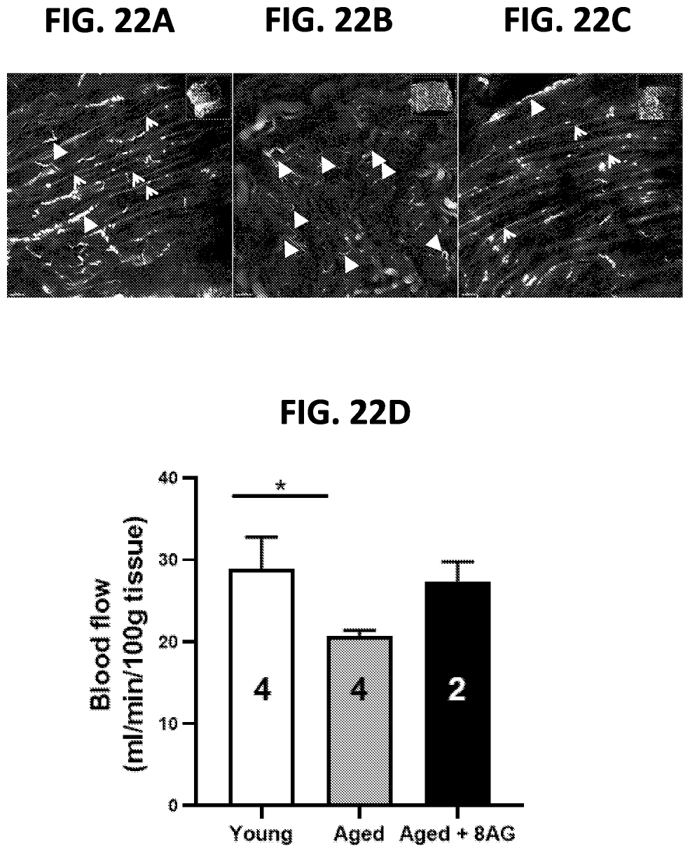

Bladders perfused with fluorescent beads showed that young bladders contained mostly straight vessels (white arrows) throughout the tissue (FIG. 22A). In contrast, aged bladders (FIG. 22B) displayed only tortuous vessels (red arrows) and in which the tissue exhibited decreased areas of perfusion and appeared ischemic (severe tortuosity may obstruct blood flow and bead injection shows perfusion). Chronic treatment of aged rats with 8-AG (FIG. 22C) reduced the number of tortuous vessels, and the tissue no longer appeared ischemic, resembling young tissue. Prior to imaging, bladders were cleared using the CUBIC method and images acquired by ribbon-scanning confocal microscopy. In addition, as shown in FIG. 22D, compared to younger rats, untreated aged rats showed a significant decrease in bladder blood flow (using Doppler flowmeter). In contrast, there was no significant difference in bladder blood flow in young versus 8-AG treated old rats (FIG. 22D).

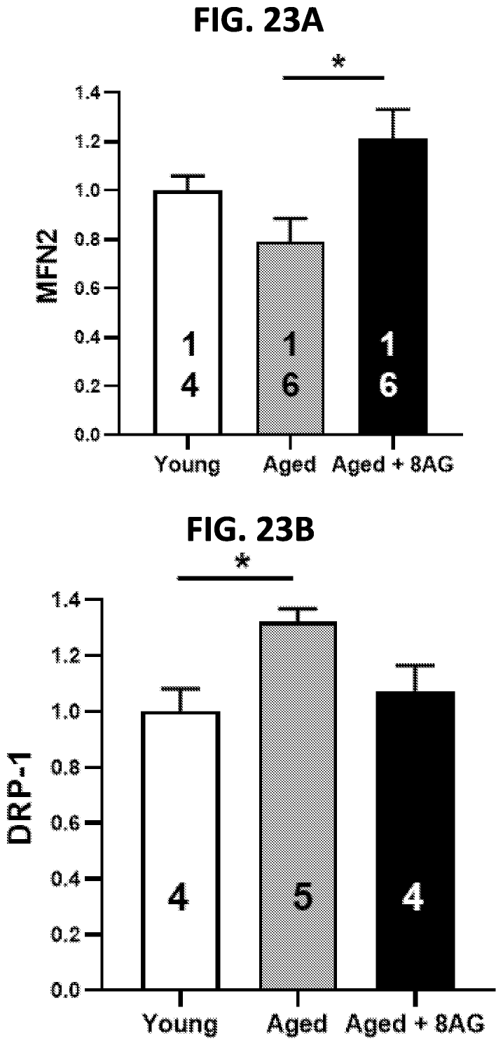

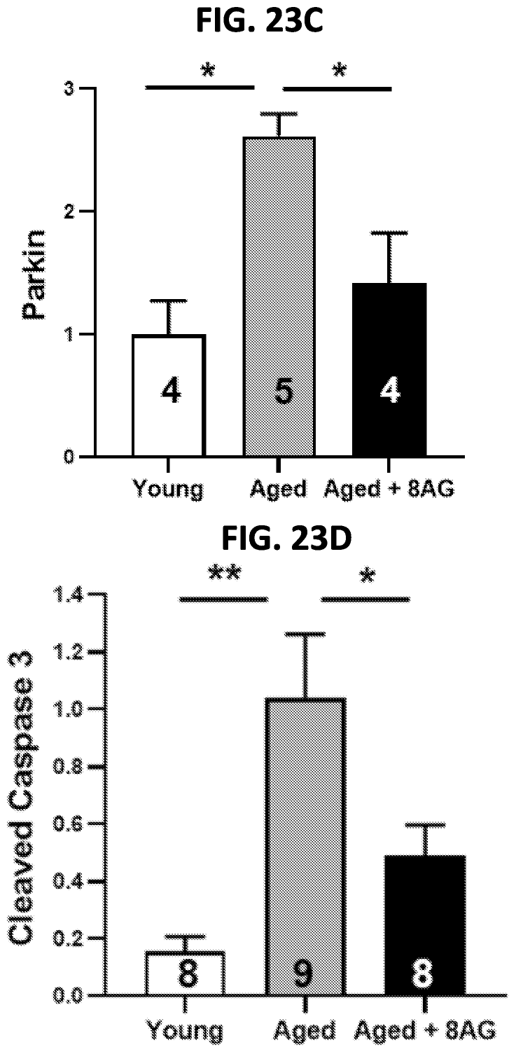

As illustrated in FIGS. 23A-23D, aging was associated with significant changes in the expression of key proteins in the bladder mucosa including the following: 1) a trend toward decreased mitofusin 2 (MFN2 normally decreases autophagy and removal of damaged mitochondria); 2) increased dynamin-related protein 1 or DRP-1 (leads to enhanced apoptosis); 3) increased caspase 3 (plays role in apoptosis); and 4) increased Parkin (which may act to remove damaged mitochondria under conditions of oxidative stress). With 8-AG treatment, these mucosal abnormalities were reversed or restored to levels similar to the younger state.

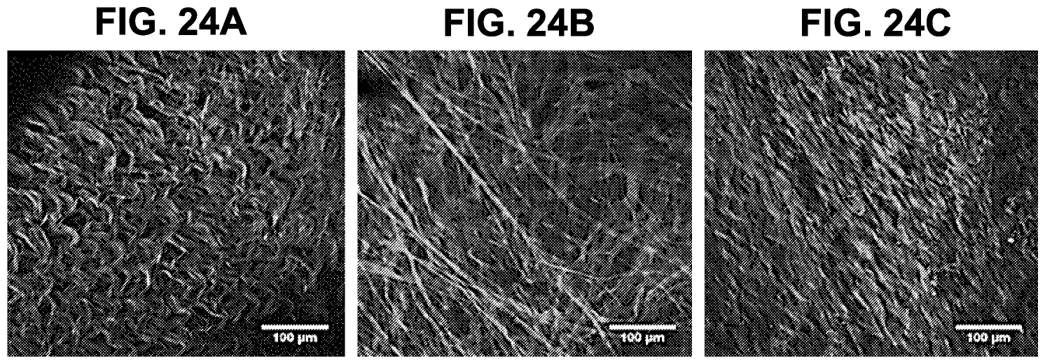

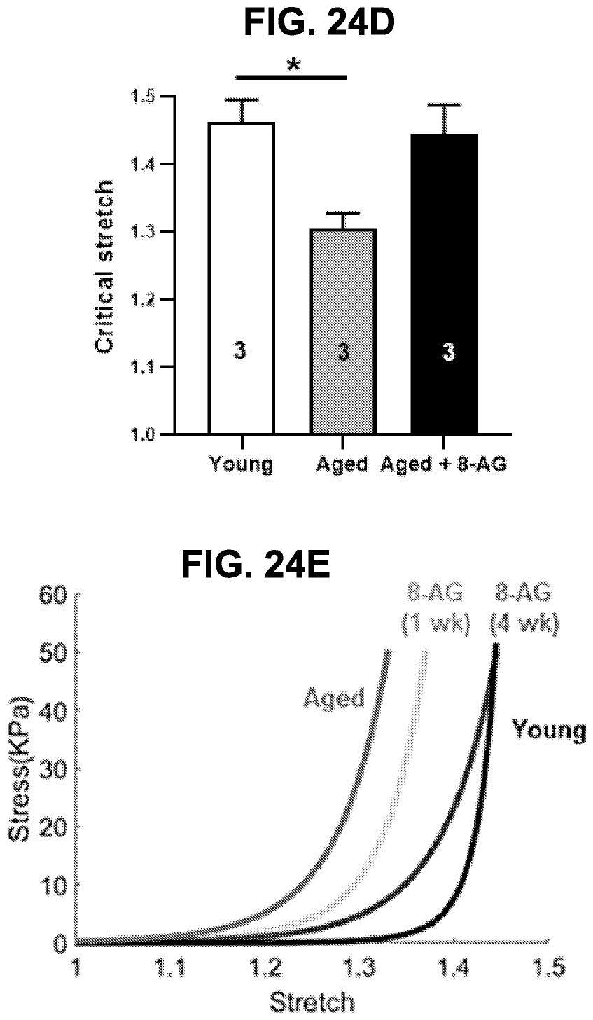

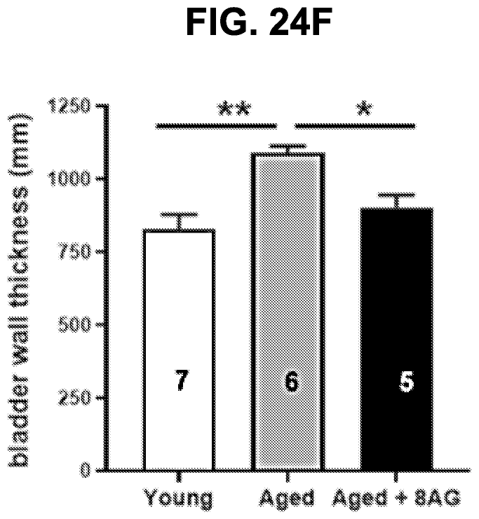

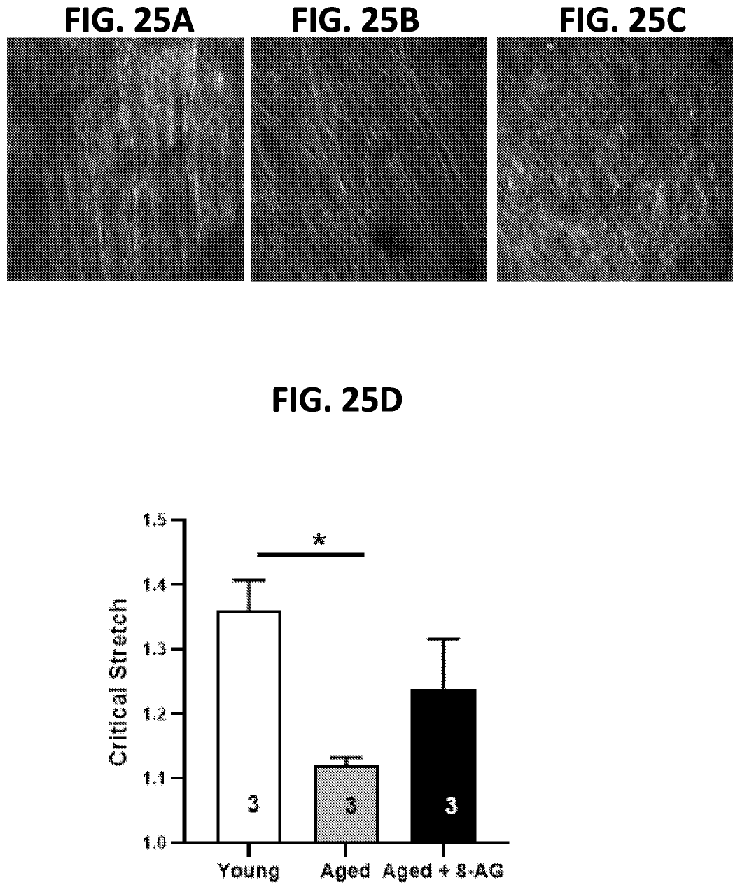

In all bladders, the mechanical reaction to mild stretch, as shown in the stress-stretch curve (FIG. 24E), was a compliant response (‘soft phase’) in which large changes in the extension of the bladder wall caused little change in stress. However, once a threshold of stretch was reached (FIG. 24E), stretch induced a noncompliant response (‘stiff phase’) in which small changes in the extension of the bladder wall caused a large change in stress. The stretch at which this steep increase in stiffness occurred depended on the tortuosity of the collagen fibers. In this regard, the tortuosity enabled bladder filling at low mechanical loads (or pressure) because fibers contributed little to load bearing until they were straightened (‘recruited’). As increasing numbers of fibers were recruited, the bladder rapidly stiffened. With age, collagen fiber tortuosity was diminished, which means that a substantial fraction of fibers was highly straightened in both the detrusor layers (compare FIG. 24A with FIG. 24B) and lamina propria. This diminished tortuosity with age resulted in a leftward shift of the bladder's stress-stretch relationship (FIG. 24E) that manifested as a substantial reduction in the stretch at which the stiff phase for bladder occurred in aged relative to young rats (FIGS. 24D and 25D). Treatment of older rats with 8-AG, restored fiber tortuosity to that of younger rats (FIGS. 24C and 25C), corresponding to a larger fraction of highly tortuous fibers which allowed increased filling at a lower pressure. This resulted in a more extensible, compliant bladder (FIG. 24E). The 8-AG treatment affected both the first and second transition points, indicating that the treatment restored collagen fiber tortuosity in the detrusor layers (FIG. 24D) and lamina propria (FIG. 25D), which resulted in a more compliant bladder closer to a younger state. In addition, bladders from aged rats exhibited a significant increase in bladder wall width (FIG. 24F), which was restored to that of a younger state by 8-AG treatment.

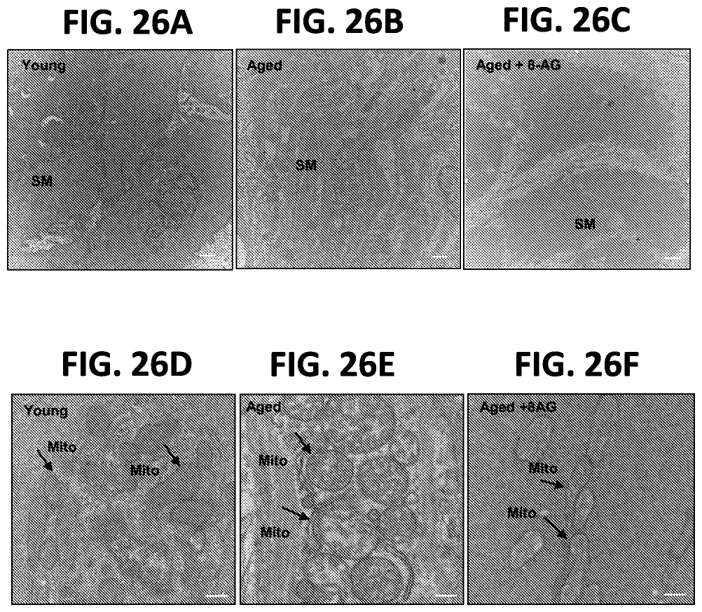

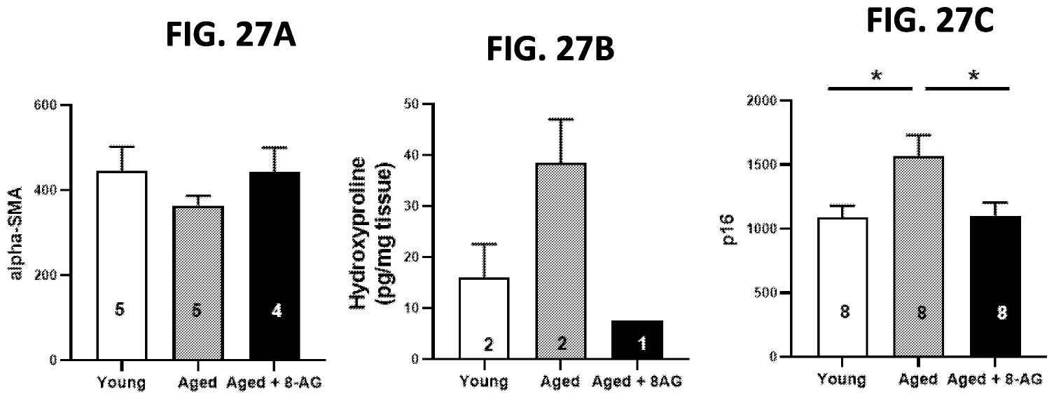

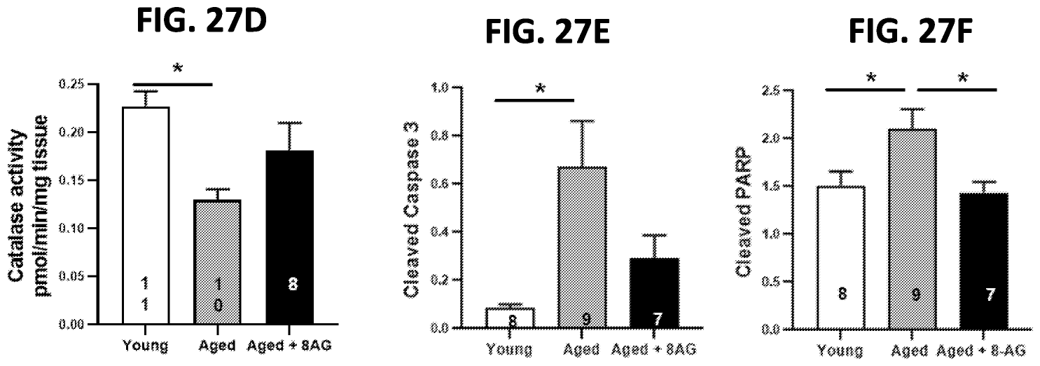

Aging was also associated with significant morphological changes in the urinary bladder smooth muscle, showing a separation and degeneration of smooth muscle cells (e.g., comparing FIG. 26A to FIG. 26B). Further, this structural pathology was accompanied by swelling and disruption of the smooth muscle mitochondria (e.g., comparing FIG. 26D to FIG. 26E). In addition, aging was associated with a trend toward decreased expression of the smooth muscle marker α-SMA (FIG. 27A) as well as hydroxyproline (FIG. 27B), which is used as an indicator of fibrosis and significant changes within the detrusor smooth muscle, the cellular senescence marker p16 (FIG. 27C), and catalase activity (FIG. 27D) which may play a role in reduced defense against ROS in aging. Further, with aging, a significant increase was observed for both cleaved caspase 3 (FIG. 27E) and cleaved PARP (FIG. 27F), both of which are involved in senescence and programmed cell death. With 8-AG treatment, smooth muscle structural anomalies (FIG. 26C), including changes to the mitochondria (FIG. 26F), were reversed. Moreover, 8-AG restored expression of hydroxyproline (FIG. 27B), the senescence marker p16 (FIG. 27C), catalase activity (FIG. 27D), cleaved caspase 3 (FIG. 27E), and cleaved PARP (FIG. 27F) to levels similar to the younger state.

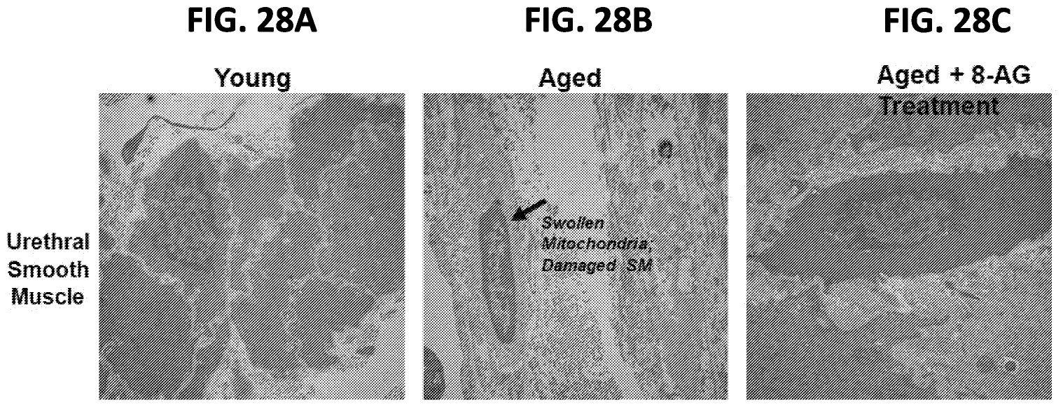

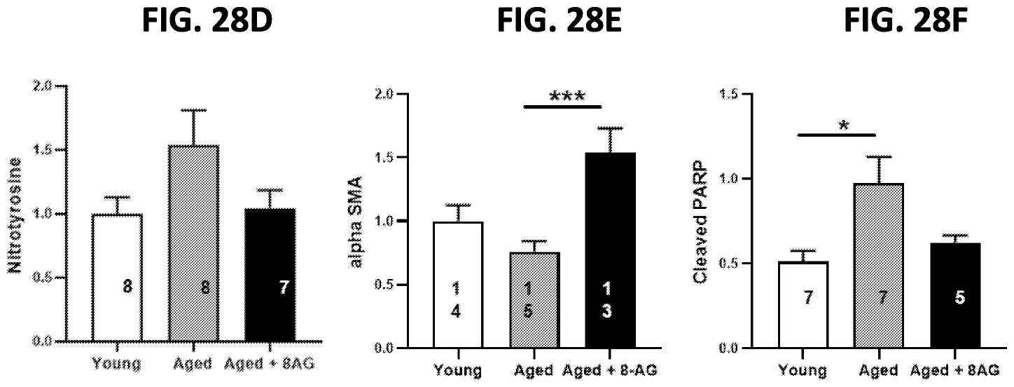

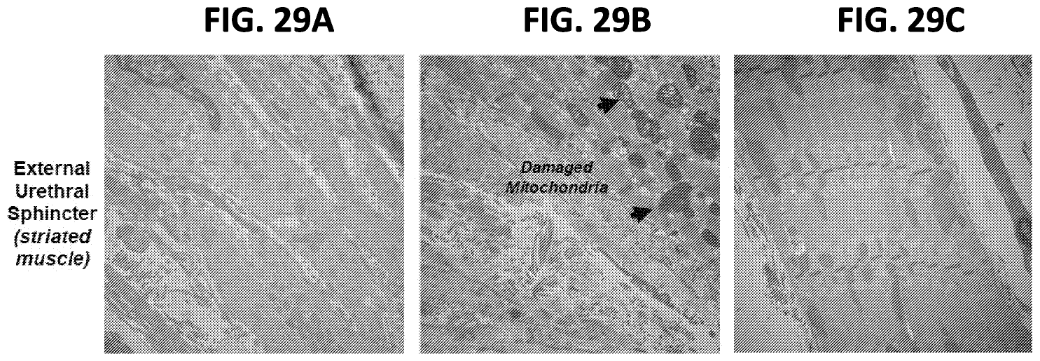

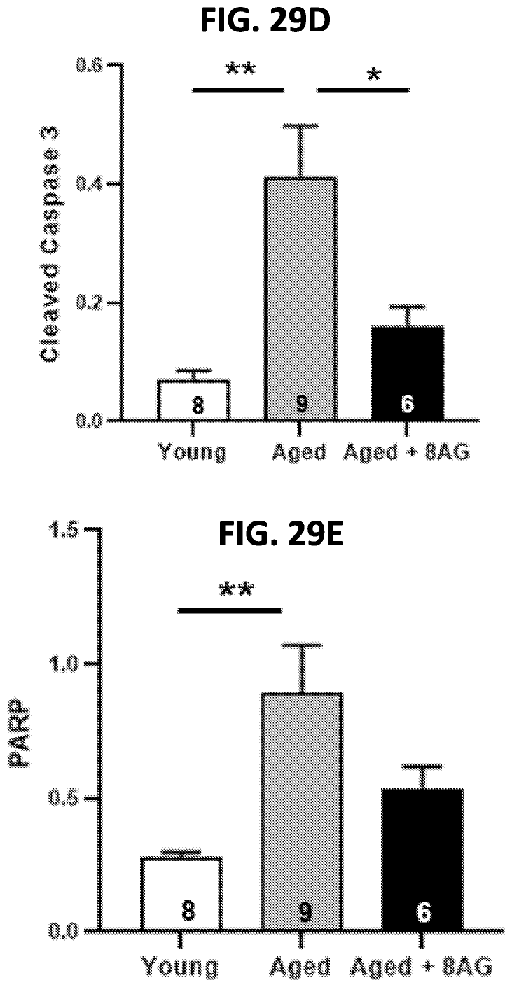

Aging was associated with morphological changes in both urethral smooth muscle (compare FIG. 28A to FIG. 28B) and urethral external sphincter striated muscle (compare FIG. 29A to FIG. 29B). These structural pathological findings were accompanied by swelling as well as disruption of the smooth and striated muscle mitochondria. With oral 8-AG treatment, both smooth (FIG. 28C) and striated (FIG. 29C) muscle abnormalities, including changes to the mitochondria, were reversed to a younger state. Consistent with the structural changes, in untreated aged rats, trends were observed toward increases in nitrotyrosine (FIG. 28D, which correlates with increased free radicals); decreases in α-SMA (FIG. 28E); and significant increases in cleaved PARP (FIG. 28F), cleaved caspase 3 (FIG. 29D), and cleaved PARP (FIG. 29E). In contrast, in aged rats treated with 8-AG, urethral levels of nitrotyrosine, α-SMA and cleaved PARP, and EUS levels of cleaved caspase 3 and cleaved PARP were similar to those in young animals.

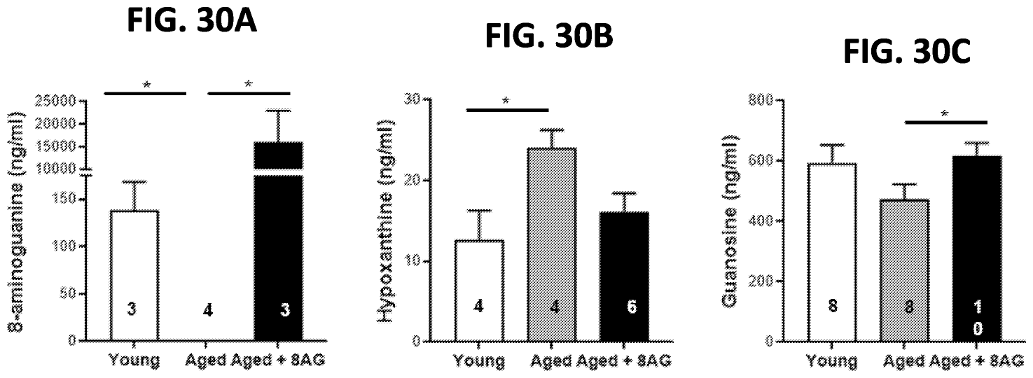

In untreated aged rats, urinary levels of endogenous 8-AG were undetectable but were restored to younger levels following oral 8-AG treatment (FIG. 30A). Further, in untreated aged rats, urinary hypoxanthine levels were higher compared with young rats, and oral 8-AG treatment reduced urinary hypoxanthine to levels comparable to younger rats (FIG. 30B). Aged rats exhibit a trend toward decreased urinary levels of guanosine (protective role in age-related diseases), yet urinary guanosine levels were similar in 8-AG-treated aged rats compared with young rats (FIG. 30C).

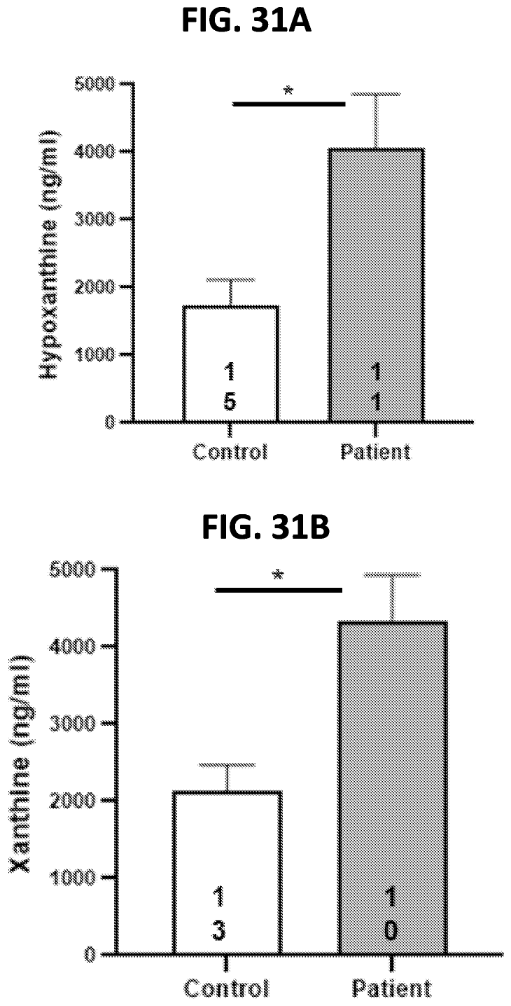

Consistent with augmented levels of uro-damaging purine metabolites in aged animals, aged urology patients exhibited significant increases in urinary levels of hypoxanthine (FIG. 31A), xanthine (FIG. 31B), and urinary symptoms compared with young healthy controls.

Thus, LUT form and function in young versus old versus 8-AG-treated old rats was compared. Compared to young rats, untreated older rats exhibit: 1) bladder voiding dysfunction; 2) decreases in tactile sensitivity to mechanical stimuli; 3) abnormal bladder vascular remodeling and reduced bladder blood flow; 4) abnormalities in both smooth and striated muscle morphology and mitochondrial structure; 5) reduced collagen fiber tortuosity; 6) increased bladder stiffness; 7) increased urinary levels of ‘uro-damaging’ hypoxanthine; and 8) decreased urinary levels of 8-AG. In contrast to young versus old rats, all LUT outcome measures were similar in young rats versus older rats treated with 8-AG, an endogenous and potent inhibitor of PNPase.

Inosine, guanosine, and hypoxanthine have not been previously shown to affect the structure of the microcirculation, to reorganize collagen fibers, or to reverse mitochondrial abnormalities. The wide-ranging effects of 8-AG in aging are surprising. These studies were performed in rats that were near the end of their life span and had already developed severe bladder pathologies that were unlikely to be reversed by treatment. However, 8-AG treatment for only 6 weeks completely or partially reversed all of the measured molecular, cellular and functional bladder abnormalities associated with aging.

### Example 7—Methods and Materials (for Example 8)

Materials: The 8-AG was purchased from Toronto Research Chemicals (Toronto, Ontario, Canada).

Animals: Female Sprague Dawley rats were employed in this example (Charles River; Wilmington, Mass.). Three groups of animals were included. Group 1 received an i.p. injection of saline and was used as a control; groups 2 and 3 received an i.p. injection of cyclophosphamide (CYP, SIGMA-ALDRICH®, St Louis, Mo., day 0, 3, 6, sacrifice day 8, 75 mg/kg, i.p.) with group 3 treated with oral 8-AG (5 mg/kg/day; starting one week prior to CYP). CYP induced bladder inflammation and is a well-established pre-clinical model for BPS/IC.

Voiding Analysis: Untreated, CYP-treated, and 8-AG-treated CYP rats were placed in metabolic cages (24 hr; once per week) for the duration of the study. The light cycle was from 7:00 AM to 7:00 PM, and food and water were provided ad libitum. Voided urine was collected in cups attached to force displacement transducers (GRASS TECHNOLOGIES®, Warwick, R.I.) connected to a computer (Windaq data acquisition software DATAQ® Instruments Inc., Akron, Ohio). Data were averaged for 24 h and analyzed for 12-h periods during the day (7:00 AM-7:00 PM) and night (7:00 PM-7:00 AM). Voiding frequency, total voided volume, and volume per void were analyzed. Voiding frequency was calculated as the number of voiding events per hour during 24 hr and during the 12-hr day and 12-hr night periods. Volume per void, which defines bladder capacity, was calculated as an average of the voids occurring during these periods.

Blood Flow Changes: Real-time bladder blood perfusion (1 mm³ tissue) was accomplished using a BLF22D laser Doppler flowmeter (Transonic Systems, Inc) with a surface probe (TypeS-APLPHS) applied to the serosal surface of the bladder (apex and neck) wall using Doppler light shift from moving RBCs to analyze flow by the Bonner algorithm. This method gives robust, non-invasive microvascular flow signals in the bladder wall of anesthetized rats.

RT-qPCR: Total RNA was isolated from bladders using TRIZOL® Reagent (THERMO FISHER SCIENTIFIC®; Waltham, Mass.) according to the manufacturer's instructions. The cDNA was synthesized using ISCRIPT™ cDNA synthesis kit (BIO-RAD®). qPCR analysis was performed using Power SYBR™ Green PCR Master Mix (THERMO FISHER SCIENTIFIC®) in the APPLIED BIOSYSTEMS® QUANTSTUDIO™ 3 Real-Time PCR System (THERMO FISHER SCIENTIFIC®). The primers used were as follows: for IL-1beta, 5′-GGGATGATGACGACCTGCTA-3′ (forward; SEQ ID NO: 1) and 5′-TGTCGTTGCTTGTCTCTCCT-3′ (reverse; SEQ ID NO: 2); for MCP-1, 5′-TGCAGAGACACAGACAGAGG-3′ (forward; SEQ ID NO: 3) and 5′-GCCAGTGAATGAGTAGCAGC-3′ (reverse; SEQ ID NO: 4); for β-actin, 5′-ACTCTTCCAGCCTTCCTTC-3′ (forward; SEQ ID NO: 5) and 5′-ATCTCCTTCTGCATCCTGTC-3′ (reverse; SEQ ID NO: 6). The threshold cycle (Ct) for target was subtracted from Ct for β-actin to give ΔCt. Results are expressed as 2AC.

Determination of mechanical pain threshold: Lower abdominal pain was evaluated using a mechanical hyperalgesia and allodynia test (Larid et al., Pain, 92:335-342, 2001; Minnet et al., PLoS One, 9:e104458, 2014). Briefly, animals were placed in individual chambers with a wire mesh floor and allowed to acclimate. This Von Frey test was performed in the lower abdomen and consists of delivering a constant pre-determined force with one of a series of Von Frey monofilaments (from 0.08 to 19.6 mN; Stoelting Co., Wood Dale, Ill., USA). Filaments were applied perpendicularly with enough strength to cause the monofilament to slightly bend. Each monofilament was tested five times, 5 s interval between each application. A response is considered positive when the animal exhibits any nocifensive behaviors such as abdomen retraction in at least three of the five filament applications. Baseline sensation measurements were taken prior to start of treatment and treatment groups were given oral 8-AG (5 mg/kg) for 1 week prior to CYP administration (75 mg/kg i.p.). Assessment of mechanical threshold was performed for control, CYP-treated, and CYP-treated with co-administration of 8-AG and animals sacrificed on day 8.

Statistics: The data were analyzed in GRAPHPAD® PRISM® 6 (GRAPHPAD®, La Jolla, Calif.) using Student's t-test and one-way ANOVA followed by appropriate post-hoc tests. P<0.05 was considered significant. The results are expressed as means±SEM.

### Example 8—Results

The LUT form and function in control versus cyclophosphamide (CYP)-treated versus CYP/8-AG-treated rats was examined. The CYP-treated rats are an art-accepted model of cystitis, and it was demonstrated that CYP-treated rats, compared to untreated controls rats, exhibit bladder voiding dysfunction, bladder inflammation, bladder hyperemia, and increased visceral pain behavior. Co-administration of 8-AG abrogated these effects of CYP; all LUT outcome measures were similar in untreated rats versus CYP-treated rats treated with 8-AG, an endogenous and potent inhibitor of PNPase. Further, in CYP-treated rats, co-administration of 8-AG suppressed bladder levels of mRNAs for the inflammatory cytokines IL-1beta and MCP-1.

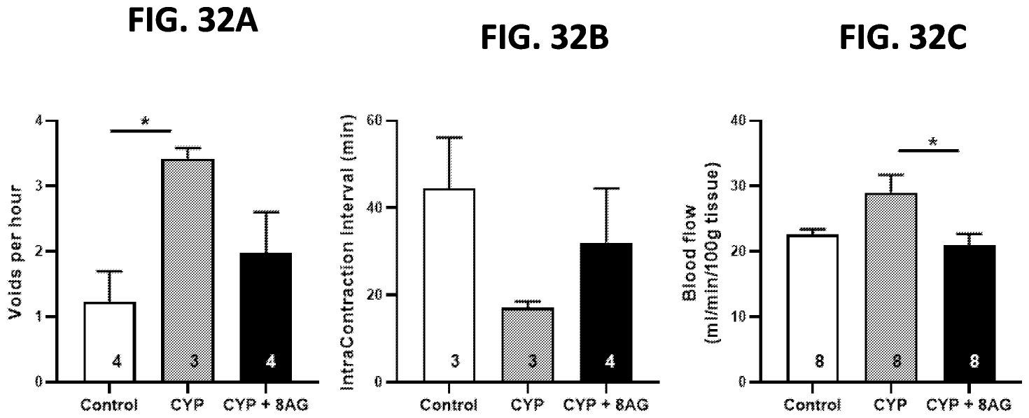

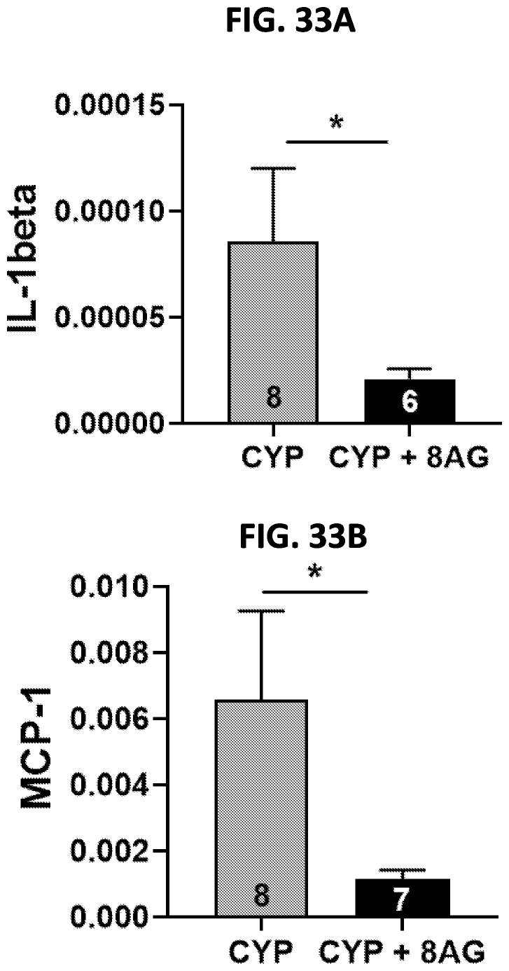

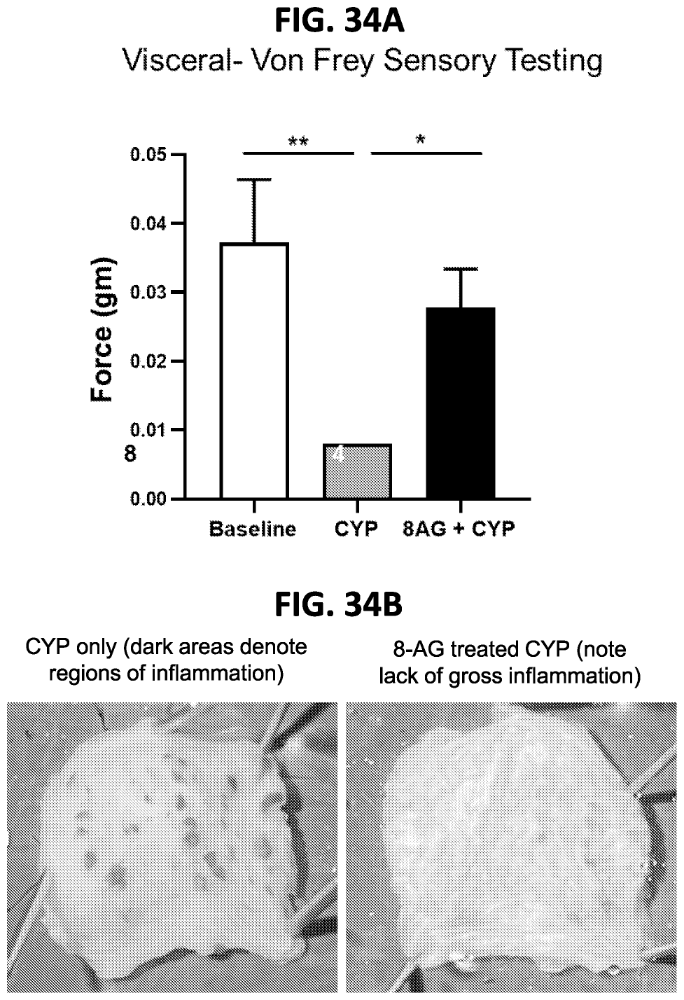

CYP-treated rats exhibited increases in voiding frequency (FIG. 32A) and decreases in the intercontraction interval (FIG. 32B). These observations are consistent with preclinical and clinical studies that show increased urinary frequency in adults diagnosed with BPS/IC and are characteristic of this syndrome. However, in CYP-treated rats that were treated chronically with 8-AG, voiding behavior was similar to that observed in untreated control rats. In addition, as shown in FIG. 32C, compared to untreated rats, the bladders of CYP-treated rats were hyperemic, which is a cardinal sign of acute inflammation. CYP-induced bladder hyperemia was abolished by 8-AG treatment (FIG. 32C). As illustrated in FIGS. 33A-33B, bladders obtained from CYP-treated rats expressed readily detectable levels of mRNA for the pro-inflammatory cytokines IL-1beta and MCP-1. However, in CYP-treated rats co-administered with 8-AG, the expression of IL-1beta and MCP-1 mRNA in the bladder were near the detection limit. IL-1beta is a cytokine that is associated with inflammation and tissue remodeling in BPS/IC,(17) and MCP-1 or monocyte chemoattractant protein-1 is a key chemokine that is associated with diverse inflammatory and chronic pain conditions.(18) As shown in FIG. 34A, the CYP group showed a decrease in mechanical pain threshold compared to normal controls and in CYP-treated rats co-administered with 8-AG, the mechanical threshold returned to near control levels. FIG. 34B depicts representative images of a CYP-treated rat bladder, which was associated with significant inflammation and bleeding. This was nearly abolished by oral treatment with 8-AG.

Thus, LUT form and function was compared in control versus CYP-treated versus CYP/8-AG-treated rats. The examples herein demonstrate that CYP-treated rats, compared to untreated controls rats, exhibit bladder voiding dysfunction, bladder inflammation, and bladder hyperemia as well as increased visceral pain behavior. Administration of 8-AG abrogated these effects of CYP. Specifically, all LUT outcome measures were similar in untreated rats versus CYP-treated rats treated with 8-AG, an endogenous and potent inhibitor of PNPase. The examples herein demonstrate that the PNPase inhibitor, 8-AG, can be used for the treatment of functional pain syndromes such as BPS/IC. In support, in CYP-treated rats, administration of 8-AG suppressed bladder levels of mRNAs for the inflammatory cytokines IL-1beta and MCP-1.

Without being bound by theory, because PNPase inhibition blocks the metabolism of inosine to hypoxanthine and guanosine to guanine, likely the uro-protective effects of PNPase inhibitors in general, and 8-AG in particular, can be mediated by increases in bladder levels of inosine and guanosine (uro-protective purines) and reductions in bladder levels of hypoxanthine (uro-damaging purine). Thus, blocking PNPase can increase ‘uro-protective’ precursors (inosine and guanosine) while simultaneously decreasing levels of ‘uro-toxic’ hypoxanthine.

8-AG had wide-ranging beneficial effects on molecular and functional abnormalities in the inflamed bladder. The wide-ranging effects of 8-AG in CYP-induced cystitis was unexpectedly superior. These studies were performed in rats that were treated with CYP, which is associated with increased bladder frequency and inflammation that were unlikely to be reversed by any treatment. The results demonstrate that 8-AG treatment for only 1 week completely or partially reverses all of the molecular and functional bladder abnormalities measured and associated with CYP-inflammation. The efficacy of 8-AG to reverse inflammation-related decrements in bladder form and function was unexpected.

It will be apparent that the precise details of the methods or compositions described may be varied or modified without departing from the spirit of the described invention. We claim all such modifications and variations that fall within the scope and spirit of the claims below.

1. A method of treating bladder or urethra dysfunction or disease in a subject, comprising:
selecting a subject with bladder or urethra dysfunction or disease; and
administering to the subject a therapeutically effective amount of a purine nucleoside phosphorylase (PNPase) inhibitor or a PNPase purine nucleoside substrate, thereby treating the bladder or urethra dysfunction or disease.

2. The method of claim 1, wherein the PNPase inhibitor is a guanine comprising a substituent at the 8-position, a guanosine comprising a substituent at the 8-position, an inosine comprising a substituent at the 8-position, a hypoxanthine comprising a substituent at the 8-position, a PNPase transition state analog, or a pharmaceutically acceptable salt thereof.

3. The method of claim 2, wherein the substituent is amine, hydroxyl, nitro, nitroso, alkoxy, carbonyl, halogen, carboxyl, ester, carbonate, amide, or haloaliphatic.

4. The method of claim 2, wherein the substituent is amine.

5. The method of claim 2, wherein the guanine comprising a substituent at the 8-position is 8-aminoguanine.

6. The method of claim 2, wherein the PNPase transition state analog is:
7-[(2S,3S,4R,5R)-3,4-dihydroxy-5-(hydroxymethyl)pyrrolidin-2-yl]-3H,4H,5H-pyrrolo[3,2-d]pyrimidin-4-one;
7-(((3R,4R)-3-hydroxy-4-(hydroxymethyl)pyrrolidin-1-yl)methyl)-3H-pyrrolo[3,2-d]pyrimidin-4(5H)-one;
7-(((2R,3S)-1,3,4-trihydroxybutan-2-ylamino)methyl)-3H-pyrrolo[3,2-d]pyrimidin-4(5H)-one;
7-((1,3-dihydroxypropan-2-ylamino)methyl)-3H-pyrrolo[3,2-d]pyrimidin-4(5H)-one; or
a pharmaceutically acceptable salt thereof.

7. (canceled)

8. The method of claim 2, wherein the PNPase transition state analog or pharmaceutically acceptable salt thereof is administered intravenously or into the bladder or the urethra of the subject.

9. The method of claim 2, comprising administering to the subject the therapeutically effective amount of the PNPase inhibitor, wherein the PNPase inhibitor is a guanine comprising a substituent at the 8-position or a guanosine comprising a substituent at the 8-position, and wherein the PNPase inhibitor is administered orally, intravenously, or into the bladder or the urethra of the subject.

10. The method of claim 1, wherein the subject has urethra dysfunction, and wherein the administration of the PNPase inhibitor or PNPase purine nucleoside substrate:
a) improves morphology of smooth or striated muscle in the urethra;
b) decreases disruption of mitochondria in the urethra; or
c) increases expression of alpha-smooth muscle actin (α-SMA) and cathepsin B in the urethra.

11. The method of claim 1, wherein:
a) the subject has bladder dysfunction, and has at least one of increased void volume, decreased void efficiency, decreased void frequency, increased bladder capacity, increased bladder storage, increased bladder wall volume, decreased sensitivity to stimuli, increased bladder ischemia, increased oxidative stress in the bladder, increased mitochondrial dysfunction in the bladder, decreased bladder contractility, or nocturia, as compared with a subject without the bladder dysfunction; or
b) the subject has urethra dysfunction, and has at least one of increased oxidative stress in the urethra, increased mitochondrial dysfunction in the urethra, or decreased urethra contractility, as compared with a subject without the urethra dysfunction.

12. (canceled)

13. The method of claim 1, wherein the subject has incontinence.

14. The method of claim 13, wherein the incontinence is stress, urgency, or spontaneous incontinence.

15-16. (canceled)

17. The method of claim 1, wherein the subject has interstitial cystitis, radiation cystitis, or bladder pain syndrome.

18. The method of claim 1, wherein administering comprises delivering the PNPase inhibitor or PNPase purine nucleoside substrate into the bladder or the urethra of the subject.

19-20. (canceled)

21. A method of increasing bladder smooth muscle contractility or bladder wall volume in a subject, comprising
selecting the subject in need of increased bladder smooth muscle contractility or decreased bladder wall volume, and
administering to the subject a therapeutically effective amount of a purine nucleoside phosphorylase (PNPase) inhibitor or PNPase purine nucleoside substrate, thereby increasing bladder smooth muscle contractility or decreasing bladder wall volume in a subject.

22-33. (canceled)

34. The method of claim 1, wherein the subject has overactive bladder.

35. The method of claim 1, wherein the subject has underactive bladder.

36. (canceled)

37. A method of improving urethral function in a subject, comprising:
selecting a subject with urethra dysfunction or disease; and
administering to the subject a therapeutically effective amount of a purine nucleoside phosphorylase (PNPase) inhibitor or a PNPase purine nucleoside substrate, and
wherein administration of the PNPase inhibitor or a PNPase purine nucleoside substrate:
a) improves the morphology of the smooth or striated muscle in the urethra;
b) decreases disruption of mitochondria in the urethra; or
c) increases expression of alpha smooth muscle actin and cathepsin B in the urethra, thereby improving urethral function in the subject.

38-45. (canceled)

46. The method of claim 1, wherein the subject has urethral stricture.

47. (canceled)

48. The method of claim 1, wherein the subject further has bladder disease, and wherein the bladder disease comprises interstitial cystitis.

49-50. (canceled)

51. The method of claim 1, wherein the subject is a human subject.

52. The method of claim 1, wherein the subject is a veterinary subject.

53. (canceled)
